(19) United States
(12) Patent Application Publication
(10) Pub. No.: US 2025/0320459 A1
(43) Pub. Date: Oct. 16, 2025

(54) FUNCTIONALIZED MOLECULAR LAYERS AND SCAFFOLDS, AND METHODS OF PREPARATION AND USE THEREOF

(71) Applicant: PURDUE RESEARCH FOUNDATION, West Lafayette, IN (US)

(72) Inventors: Shelley Claridge, Lafayette, IN (US); Juan Arango, Lafayette, IN (US); Luis Solorio, West Lafayette, IN (US); Sarah Libring, West Lafayette, IN (US); Anamika Singh, West Lafayette, IN (US)

(21) Appl. No.: 18/863,206

(22) PCT Filed: May 5, 2023
(86) PCT No.: PCT/US2023/066713

§ 371 Date: Nov. 5, 2024

Related U.S. Application Data
(60) Provisional application No. 63/338,464, filed on May 5, 2022.
(60) Provisional application No. 63/347,335, filed on May 31, 2022.
(60) Provisional application No. 63/396,097, filed on Aug. 8, 2022.
(60) Provisional application No. 63/396,093, filed on Aug. 8, 2022.
(60) Provisional application No. 63/396,086, filed on Aug. 8, 2022.
(60) Provisional application No. 63/396,079, filed on Aug. 8, 2022.

(51) Int. Cl. C12N 5/077

(52) U.S. Cl. CPC C12N 5/0658; C12N 2533/30; C12N 2535/10

(57) ABSTRACT

Functionalized molecular layers comprising a backbone of polymerized amphiphiles for placement on a surface of a scaffold, such as a hydrogel. Methods of preparing such molecular layers and hydrogels comprising the same are also provided, as are methods of use thereof.

### PRIORITY

This application is related to and claims the priority benefit of U.S. Provisional Patent Application No. 63/338,464 filed May 5, 2022; U.S. Provisional Patent Application No. 63/347,335 filed May 31, 2022; U.S. Provisional Patent Application No. 63/396,079 filed Aug. 8, 2022; U.S. Provisional Patent Application No. 63/396,086 filed Aug. 8, 2022; U.S. Provisional Patent Application No. 63,396,093 filed Aug. 8, 2022; and U.S. Provisional Patent Application No. 63/396,097 filed Aug. 8, 2022. The contents of the aforementioned applications are hereby incorporated by reference in their entireties into this disclosure.

### GOVERNMENT RIGHTS

This invention was made with government support under CHE 2108966 awarded by the National Science Foundation. The government has certain rights in the invention.

### TECHNICAL FIELD

The present disclosure relates to a nanoscale, patterned monolayer surface for placement on a substrate comprising a polymeric material, wherein the surface can be functionalized. Methods for functionalizing the monolayer surface are also provided, as well as methods for producing the monolayer, transferring the monolayer to a scaffold (e.g., a hydrogel), and adhering cells or other targets to a polymeric material.

### BACKGROUND

Nanoscale control over surface functionality is important in applications ranging from nanoscale electronics to regenerative medicine. For example, multivalent interactions between carbohydrates (CHOs) and proteins can enable a broad range of selective interactions of critical biological importance.

Similarly, controlling the chemistry of hydrogels can be beneficial in numerous applications ranging from chromatographic separations to the design of cell culture supports and implants for regenerative medicine. Slaughter et al., Hydrogels in regenerative medicine, Advanced Materials 21: 3307-3329 (2009); Caliari & Burdick, A practical guide to hydrogels for cell culture, Natural Methods 13: 405-414 (2016); Madl & Heilshorn, Engineering hydrogel microenvironments to recapitulate the stem cell niche, Annual Review Biomedical Engineering 20: 21-47 (2018). In some cases, the chemistry of a hydrogel can be altered throughout the hydrogel via the use of new monomers. However, achieving local changes to hydrogel chemistry are increasingly important. Such changes can include the generation of gradient gels to improve chromatographic separation or microscale chemical environments in which local chemistry and mechanical properties can impact interactions with cells. Nichols et al., Nondenaturing polyacrylamide gradient gel electrophoresis, Methods Enzymology 128: 417-431 (1985-1986); Bjellqvist et al., A nonlinear wide-range immobilized pH gradient for two-dimensional electrophoresis and its definition in a relevant pH scale, Electrophoresis 14: 1357-1365 (1993); Yang et al., Spatially patterned matrix elasticity directs stem cell fate, Proc. Natl. Academy Science USA 113: 4439-4445 (2016).

Despite its advantages, controlling structure at soft interfaces is particularly challenging, due to the amorphous, often porous structure of such materials. For instance, in polymeric materials commonly used as scaffolds for cell culture, pore diameters in the range of hundreds of nanometers mean that much of the surface is recessed. This creates challenge in functionalizing surfaces to interact with larger objects such as cells, since there are a relatively limited number of sites on the ‘surface’ that maximize steric accessibility for an object larger than the pore. Furthermore, in vitro control over placement of multiple CHO groups, for example, at a nanometer length scale to allow for controlled multivalent interactions is also a significant challenge. Nanometer-resolution functional patterns are difficult to achieve in broadly used monolayer chemistries such as functional alkylthiol monolayers on gold or functional alkylsilanes on silicon.

Consequently, high resolution chemical patterning of hard, crystalline surfaces is more common. Molecular ordering relative to the inorganic substrate (e.g., Au(111), HOPG) can be used to generate structure at some nanometer scales, while molecules can be delivered to surfaces in microscale patterns using microcontact printing, or in some cases nanoscopic patterns using scanning probe lithography. Love et al., Self-assembled monolayers of thiolates on metals as a form of nanotechnology, Chem Review 105: 1103-1169 (2005); Goronzy et al., Supramolecular assemblies on surfaces: Nanopatterning, functionality and reactivity, ACS Nanoparticles 12: 7445-7481 (2018); Xia et al., Softe Lithography, Annual Review Material Sciences 28: 153-184 (1998); Saaverdra et al., Hybrid strategies in nanolithography, Rep. Prog. Phys. 73 (2010); Piner et al., “Dip-Pen” nanolithography, Science 283: 661-663 (1999); and Huo et al., Polymer pen lithography, Science 321: 1658-1660 (2008).

Soft surfaces, however, can benefit from high-resolution chemical patterning, such as ligand clustering, which can be important in cell adhesion and other applications. Molecular patterns noncovalently adsorbed on HOPG can be assembled using an internal polymerizable group to ‘set’ the molecular pattern, then covalently transferred to a soft surface such as polydimethylsiloxane (PDMS) or polymeric material (e.g., polyacrylamide (PAAm)). Bang et al., Sitting phases of polymerizable amphiphiles for controlled functionalization of layered materials, J American Chemistry Soc'y 138: 4448-4457 (2016); Villarreal et al., Modulating wettability of layered materials by controlling ligand polar head group dynamics, J American Chemistry Soc'y 139: 11973-11979 (2017); Davis et al., One nanometer wide functional patterns with a sub-10 nanometer pitch transferred to an amorphous elastomeric material, ACS Nano 15: 1426-1435 (2021); Williams et al., Designing interfacial reactions to achieve nanometer-resolution surface patterning of amorphous polymer networks, ACS Applied Materials & Interfaces: DOI: 10.1021/acsami.2c22646 (2023)(in preparation); Arango et al., Nanostructured surface functionalization of polyacrylamide polymeric materials below the length scale of polymeric material heterogeneity, ACS Applied Materials & Interfaces 14: 43937-43945 (2022); Bechtold et al., Striped poly(diacetylene) monolayers control adsorption of polyelectrolytes and proteins on 2D materials and elastomers, ACS Applied Nano Materials 4: 7037-7046 (2021); Williams et al., Control of c2c12 murine myoblast adhesion and differentiation by hierarchically patterned elastomeric surfaces (2022)(in preparation); Signh et al., Controlling the nanoscale clustering of carbohydrates to understand multivalent carbohydrate-lectin interactions (2022)(in preparation).

In certain embodiments, the hydrogel comprises PAAm. PAAm is a hydrogel that is widely used in protein and deoxyribonucleic acid (DNA) chromatography, cell culture, and other applications. Nichols et al. (1985-1986), supra; Pandey & Mann, Proteomics to study genes and genomes, Nature 405: 837-846 (2000); Engler et al., Matrix elasticity directs stem cell lineage specification, Cell 126: 677-689 (2006). Gel formation is typically achieved through radical-mediated polymerization of an acrylamide (Aam) monomer with a bis-acrylamide (Bis) crosslinker. Pore sizes, and thus mechanical properties, can be controlled by adjusting the ratio of acrylate to water (% T), and the ratio of crosslinker to monomer (% C) as shown in FIG. 1A. PAAm networks exhibit significant hierarchical complexity at mesoscopic scales (ca. 200-500 nm); thus, exerting high-precision control over the surface chemistry of polyacrylamide and similar hydrogels has traditionally been challenging.

Notwithstanding the challenges traditionally present with PAAm hydrogels, highly ordered arrays of striped polydiacetylenes (sPDAs) can be generated, with the relatively rigid PDA maintaining the chemical patterns at the surface of the soft material. However, sPDA transfer depends, to some extent, on long-range ordering on HOPG, and on achieving high conversion from monomeric diacetylene to polydiacetylene. Shi et al., Plenty of room at the top: A multi-scale understanding of nm-resolution polymer patterning on 2D materials, Angew Chemistry, Int'l Ed. 60: 25436-25444 (2021); Shi et al., Nanometer-scale precision polymer patterning of PDMS: multiscale insights into patterning efficiency suing alkyldiynamines, ACS Applied Materials & Interfaces 14: 22634-22642 (2022). Conventional techniques place significant constraints on the choice of head group chemistry. Accordingly, it would be useful to be able to choose monolayer head group chemistry and append secondary surface chemistry. It is also an object of the present disclosure to provide biocompatible substrates, such as hydrogels, with ordered patterns of functional groups on the surface thereof.

Furthermore and similarly, all living cells are decorated by a dense and complex array of glycans which are expressed on cell surfaces in various forms (e.g., free oligosaccharides, glycoproteins, glycolipids and/or proteoglycans). Cell surface glycans are recognized by glycan binding proteins (GBPs), also referred to as lectins. The glycan-lectin interaction mediates or modulates many cellular interactions (e.g., influenza A virus (IAV) infections, cell adhesion, differentiation and tumor metastasis). Koehler et al., Initial step of virus entry: virion binding to cell-surface glycans, Annual Review of Virology 7: 143-165 (2020). Hence, understanding glycan-lectin interactions at the molecular level is important for the development of diagnostic and therapeutic tools.

One challenge in replicating glycan-lectin interactions occurring in vivo is the relatively weak binding (dissociation constants in the millimolar range) between glycan monomers (i.e. monosaccharides) and lectins. Biological systems have evolved to take advantage of this weak interaction by incorporating multiple receptors in each lectin. Multivalent glycan-lectin interactions increase binding strength, shifting the binding dissociation constant from the millimolar to the nanomolar range, and improve selectivity for specific glycans. In biological systems, such multivalent interactions occur at the nm scale. For example, the diameter of a typical hemagglutinin lectin expressed on the IAV surface is ca. 14 nm; similarly, the targeted glycan length is ca. 2-20 nm. Overeem et al., A dynamic, supramolecular view on multivalent interaction between influenza virus and host cells, Small 17 (2021). Therefore, to mimic multivalent interactions that occur in vivo, controlling the presentation of glycans at scales from 1-20 nm is crucial.

In view of the above, it would also be useful to provide a scalable and broadly applicable method to generate nanoscale patterns of reactive (e.g., chemically or biologically reactive or interactive) moieties on polymeric material surfaces through post-functionalization click reactions. This and other objects and advantages, as well as inventive features, will be apparent from the detailed description provided herein.

### SUMMARY

Scaffolds are provided that comprise a molecular layer positioned on a surface of a support material. The molecular layer can comprise polymerized amphiphiles organized in a striped pattern to form an exposed polymer backbone, wherein each of the polymerized amphiphiles comprise one or more exposed alkyl chains and one or more functional head groups covalently bonded with an exposed alkyl chain. The one or more functional head groups can be in an orientation capable of binding a target and the alkyl chains and functional head groups can be organized in a repeating striped pattern comprising a width at a periodicity.

The polymerized amphiphiles of the molecular layer can be organized in a lying-down orientation (e.g., the striped pattern) substantially parallel to the surface of the support material, whereas the one or more exposed alkyl chains and the one or more functional head groups are not so restricted.

The support material can comprise an amorphous, soft, and/or porous substrate. The support material can comprise a hydrogel. The support material can comprise a hydrophilic hydrogel. The support material can comprise a polyacrylate, polyacrylamide, or polyethylene glycol (PEG) hydrogel. The support material can be compatible with a traditional cell culture support.

The molecular layer is, in certain embodiments, covalently bonded to the surface of the support material. The molecular layer can comprise a monolayer.

The periodicity of the pattern can be, in certain embodiments, tunable by adjusting a length of the one or more alkyl chains. Each of the one or more exposed alkyl chains can be a nonpolar alkyl chain.

The amphiphiles can be polymerized through polymerizable functional groups within the one or more alkyl chains. The polymerizable functional groups can be or comprise polymerizable diacetylene (DA) groups.

The one or more functional head groups can comprise chemically or biologically reactive or interactive head groups. The one or more functional head groups can be polar head groups. The one or more functional head groups can comprise carbohydrate (CHO) head groups, a carboxylate, an amine, an alcohol, a peptide, a substrate of an enzyme, or any combination of two or more of the foregoing. Where the head groups comprise CHO, the CHO type can be selected, and the pattern can be designed, such that the CHOs act together as a component of an extracellular matrix (ECM). The one or more functional head groups can comprise a monosaccharide, a glycan, a N-acetyl-D-glucosamine (GlcNAc), a glucuronic acid (GlcA), a N-acetyl-D-neuraminic acid, or any combination of two or more of the foregoing.

The polymerized amphiphiles can comprise C₅-C₃₀ functionalized amphiphiles with an internal diyne. The polymerized amphiphiles can comprise 10,12-tricosadiynamine (10,12-TCD-NH₂), 4,6-tricosadiynamine (4,6-TCD-NH₂), 10,12-pentacosadiynamine (10,12-PCD-NH₂), 4,6-pentacosadiynamine (4,6-PCD-NH₂), or any combination of the foregoing. The amphiphiles can comprise 10,12-tricosadiynol (10,12-TCD-OH), 10,12-pentacosadiynol (10,12-PCD-OH), 10,12-tricosadiynoic acid (10,12-TCD-COOH), 10,12-pentacosadiynoic acid (10,12-PCD-COOH), 4,6-tricosadiynol (4,6-TCD-OH), 4,6-pentacosadiynol (4,6-PCD-OH), 4,6-tricosadiynoic acid (4,6-TCD-COOH), 4,6-pentacosadiynoic acid (4,6-PCD-COOH), 1,2-bis(10,12-tricosadiynoyl)-sn-glycero-3-phosphocholine (diyne PC), 1,2-bis(10,12-tricosadiynoyl)-sn-glycero-3-phosphoethanolamine (diyne PE), or any combination of the foregoing.

The repeating elements of the striped pattern can be linear and substantially parallel to each other. The pattern of the one or more functional head groups can have a sub-10-nm scale. The width of the pattern of functional head groups can be at or about 1 nm (e.g., 1 nm). The pattern of functional head groups can be parallel lines.

The pattern of the one or more functional head groups can, in some instances, mimic properties of macromolecules or components of an ECM useful for modulating cell adhesion, cell proliferation, cell differentiation, and/or reprogramming of a cell. The pattern of the one or more functional head groups can mimic properties of polysaccharide components. For example, and without limitation, the one or more functional head groups can comprise CHO head groups and the pattern of CHO head groups can mimic hyaluronic acid.

In certain embodiments, the molecular layer comprises a multi-valent binding surface.

The target can be a cell, a biomolecule, or a bioactive molecule. The target can be a myoblast.

In certain embodiments, individual ordered regions of the polymerized amphiphiles have linear dimensions of greater than about 100 nm in length while maintaining nanoscale periodicity of the striped pattern of functional head groups. The individual ordered regions of the polymerized amphiphiles can have linear dimensions of greater than about 10 μm (e.g., 10 μm, 11 μm, 12 μm, or 15 μm) in length while maintaining nanoscale periodicity of the striped pattern of functional head groups. In certain embodiments, the individual ordered regions of the polymerized amphiphiles have linear dimensions of greater than about 1 μm (e.g., 1 μm, 2 μm, 3 μm, or 4 μm) in length while maintaining nanoscale periodicity of the striped pattern of functional head groups.

The periodicity can be about 5 nm to about 10 nm (e.g., 5-10 nm). The periodicity can be about 5 nm to about 7 nm (e.g., 5-7 nm).

The support material can have a pore diameter from about 1 nm to about 200 nm (e.g., 1-200 nm).

The exposed polymer backbone can be covalently bonded to an azide-functionalized molecule or a thiol-functionalized molecule in the absence of copper. In certain embodiments, the azide-functionalized molecule or the thiol-functionalized molecule is a cell adhesion molecule. The cell adhesion molecule can be cyclic arginine-glycine-aspartic acid (RGD).

A functionality of the molecular layer can comprises CHO, one or more peptides, a tripeptide sequence of RGD or a functional analog thereof, a matrisome component, or a combination of any of the foregoing.

The scaffolds and molecular layers hereof can be for use in cell culturing, chromatographic biomolecule separation, modulating microscale chemical environments, as a regenerative medicament or implant, or any combination of the foregoing.

Cell or tissue culture systems are also provided, such systems comprising any of the scaffolds described herein. Also provided are multivalent receptor assays comprising any of the scaffolds described herein. In certain embodiments, the multivalent receptor assay is or comprises a lectin assay.

Still further, methods of preparing a hydrogel are provided. In certain embodiments, a method of preparing a hydrogel comprising a functionalized surface comprises: obtaining a first substrate comprising a molecular layer positioned thereon, the molecular layer comprising: polymerized amphiphiles organized in a lying-down orientation to form an exposed polymer backbone, each of the polymerized amphiphiles comprising one or more alkyl chains and one or more functional head groups covalently bonded with an alkyl chain, wherein the one or more functional head groups are in an orientation capable of binding a target and the alkyl chains and one or more functional head groups are organized in a repeating striped pattern comprising a width at a periodicity; and transferring the molecular layer from the first substrate to a surface of a hydrogel by covalent binding, whereupon the surface of the hydrogel comprises the molecular layer and is functionalized with the one or more functional head groups.

The pattern of the one or more functional head groups can be dictated by organization of the polymerized amphiphiles within the exposed polymer backbone. The one or more alkyl chains can lie flat on the first substrate following polymerization, but can be exposed after transferring the molecular layer to the surface of the amorphous and/or soft support material. In certain embodiments, the method further comprises exfoliating the hydrogel from the first substrate.

The hydrogel can be amorphous, soft and/or porous.

Transferring the molecular layer to a surface of an amorphous and/or soft support material can be performed concurrently with curing the amorphous and/or soft support material. Transferring the molecular layer to a surface of an amorphous and/or soft support material can be performed subsequently to curing the amorphous and/or soft support material.

In certain embodiments, transferring the molecular layer to a surface of an amorphous and/or soft support material is performed concurrently with curing the amorphous and/or soft support material and the method further comprises: applying on top of the molecular layer an aqueous reaction mixture comprising a monomer, a crosslinker, and a radical initiator, wherein the aqueous reaction mixture can undergo free radical polymerization to form a hydrogel; and allowing the aqueous reaction mixture to cure by free-radical polymerization to form the hydrogel.

The hydrogel can comprise available reaction sites and/or a thin layer of crosslinker on the surface of the hydrogel to which the molecular layer is to be transferred.

The aqueous reaction mixture can further comprises a radical stabilizer.

Obtaining a first substrate comprising a molecular layer positioned thereon can comprise: ordering polymerizable amphiphiles as a standing phase film on an aqueous subphase, each of the polymerizable amphiphiles comprising one or more alkyl chains comprising a polymerizable functional group and one or more functional head groups covalently bonded with an alkyl chain; contacting a first substrate to the film in a manner that reorders the polymerizable amphiphiles from the standing phase into ordered a repeating striped pattern on the first substrate, wherein the ordered pattern forms a polymer backbone in which the polymerizable amphiphiles are organized in a lying-down orientation and define the pattern of the one or more functional head groups; and polymerizing the polymerizable functional groups of the polymerizable amphiphiles with polymerizable functional groups of adjacent amphiphiles to stabilize the pattern and polymerize the amphiphiles.

The aqueous reaction mixture can comprise an acrylamide monomer or a diacrylamide monomer. The crosslinker can be bisacrylamide, the radical initiator is ammonium persulfate, and/or the radical stabilizer can be N,N,N′,N′-tetramethylethylenediamine. In certain embodiments, the reaction mixture comprises from about 10% to about 50% total acrylamide (e.g., 10% to about 50%, about 10% to 50%, or 10% to 50%) and from about 1% to about 10% bisacrylamide (e.g., 1% to about 10%, about 1% to 10%, or 1% to 10%), as a percentage of total acrylamide in each instance.

The polymerizable functional groups can comprise DA groups and polymerization thereof with the DA groups of adjacent amphiphiles forms a polydiacetylene within the exposed polymer backbone. The first substrate can be HOPG, graphene, or a layered material comprising MoS₂ or WS₂.

In certain embodiments, the method can further comprise: after applying the aqueous reaction mixture on top of the patterned layer of polymerized amphiphiles, applying a second substrate, which can be the same as or different from the first substrate, on top of the aqueous reaction mixture, whereupon after the aqueous reaction mixture is allowed to cure, the hydrogel is also covalently bonded to the second substrate. The second substrate can comprise, on its surface, a layer of reactive functional groups or a patterned layer of polymerized amphiphiles, which can be the same as or different from the patterned layer of polymerized amphiphiles of the first substrate. The second substrate can comprise glass or vinyl-functionalized glass. The first and second substrates can comprise the same material selected from the group consisting of HOPG, graphene, and a layered material comprising MoS₂ or WS₂. The layer of reactive functional groups can comprise one or more alkenes. In certain embodiments, the surface of the second substrate comprises the layer of reactive functional groups or a patterned layer of polymerized amphiphiles faces the aqueous reaction mixture when the second substrate is applied on top of the aqueous reaction mixture.

The pattern of the functional head groups can be parallel lines.

The one or more functional head groups can be chemically or biologically reactive or interactive head groups. The chemically or biologically reactive or interactive head groups can be cationic under physiological conditions. In certain embodiments, the cationic head group is an amine. The one or more functional head groups can comprise CHO head groups. The periodicity can be about 5 nm to about 6 nm (e.g., about 5 nm to 6 nm, 5 nm to about 6 nm, or 5 nm to 6 nm). The hydrogel can have a pore diameter from about 1 nm to about 200 nm (e.g., about 1 nm to 200 nm, 1 nm to about 200 nm, or 1 nm to 200 nm). The CHO head groups can be GlcNAc or GlcA.

The amphiphiles can comprise C₅-C₃₀ functionalized amphiphiles with an internal diyne. The amphiphiles can comprise TCD-NH₂, PCD-NH₂, or a combination of the foregoing. The amphiphiles can comprise 10,12-TCD-GlcNAc, 4,6-TCD-GlcNAc, TCD-OH, PCD-OH, TCD-COOH, PCD-COOH, diyne PC, diyne PE, or any combination of the foregoing. The chemically or biologically reactive or interactive head groups can be neutral or zwitterionic under physiological conditions. The neutral head group can be a hydroxyl or the zwitterionic head group can be phosphoethanolamine. The chemically or biologically reactive or interactive head groups can be anionic under physiological conditions. The anionic one or more head groups can be carboxylate.

In certain embodiments, the method can further comprise contacting the exposed polymer backbone on the surface of the hydrogel with a thiol-functionalized or an azide-functionalized molecule under conditions that promote a click reaction between a polydiacetylene of the exposed polymer backbone and a thiol of the thiol-functionalized molecule or between a polydiacetylene of the exposed polymer backbone and an azide of the azide-functionalized molecule.

The hydrogel can be polyacrylamide. The thiol-functionalized molecule or the azide-functionalized molecule can be a cell adhesion molecule. The cell adhesion molecule can be cyclic RGD.

Chromatography platforms are also provided, wherein such platforms comprise a scaffold hereof. An implant is provided comprising a scaffold hereof A cell culture platform is provided comprising a scaffold hereof. In certain embodiments, the cell culture platform is for in vitro cell culture and can be, for example, selected from the group consisting of a 3D cell culture platform, an organ-on-a-chip platform, an immune cell culture platform, and an induced pluripotent stem cell culture platform.

A method of adsorbing a biomolecule in a sample is also provided. The method of adsorbing a biomolecule in a sample can comprise contacting, under adsorptive conditions, a sample with a surface of a scaffold hereof; whereupon the biomolecule in the sample is adsorbed onto the molecular layer of the scaffold. The support material can be a polyacrylamide hydrogel. The amphiphile can comprise an amine functional head group. The amphiphile can be TCD-NH₂ or PCD-NH₂. The biomolecule can be DNA.

Still further, a method of adhering cells to a polymeric material is provided. In certain instances, such a method of adhering cells to a polymeric material can comprise contacting cells with a polymeric material comprising a scaffold hereof, wherein the exposed polymer backbone of the molecular layer comprises polydiacetylene covalently bonded to a thiol-functionalized or an azide-functionalized molecule in the absence of copper. The azide-functionalized or thiol-functionalized molecule can be is a cell adhesion molecule. The cell adhesion molecule can be a cyclic RGD. The cells can be a mixture of cells. The cells can form a tissue.

Also provided is a method of further functionalizing the surface of a polymeric material on the surface of which is a patterned layer of polymerized amphiphiles. The method comprises: obtaining a polymeric material comprising a patterned layer of polymerized amphiphiles, each of which comprises a functional head group (e.g., a chemically or biologically reactive or interactive head group) covalently bonded to an alkyl chain comprising a polymerizable diacetylene group, which polymerizes with another diacetylene group of an adjacent amphiphile to form a polydiacetylene, by which the amphiphiles are polymerized, wherein the alkyl chains lie flat on the first substrate and are positioned to form a pattern of the chemically or biologically reactive head groups about 1 nm in width at a periodicity from about 5 nm to about 10 nm, and contacting the polymeric material with a thiol-functionalized molecule under conditions that promote a click reaction between a polydiacetylene and the thiol of the thiol-functionalized molecule. The pattern can be parallel lines. The periodicity can be about 5 nm to about 7 nm. The polymeric material can have a pore diameter from about 1 nm to about 200 nm. The polymeric material can be polyacrylamide. The amphiphile can be a C₅-C₃₀ amine-functionalized amphiphile with an internal diyne, the position of which can vary (see, e.g., U.S. Pat. No. 10,889,669), such as 10,12-tricosadiynamine (10,12-TCD-NH₂), 10,12-pentacosadiynamine (10,12-PCD-NH₂), 10,12-tricosadiynol (10,12-TCD-OH), 10,12-pentacosadiynol (10,12-PCD-OH), 10,12-tricosadiynoic acid (10,12-TCD-COOH), 10,12-pentacosadiynoic acid (10,12-PCD-COOH), 4,6-tricosadiynamine (4,6-TCD-NH₂), 4,6-pentacosadiynamine (4,6-PCD-NH₂), 4,6-tricosadiynol (4,6-TCD-OH), 4,6-pentacosadiynol (4,6-PCD-OH), 4,6-tricosadiynoic acid (4,6-TCD-COOH), 4,6-pentacosadiynoic acid (4,6-PCD-COOH), 1,2-bis(10,12-tricosadiynoyl)-sn-glycero-3-phosphocholine (diyne PC), 1,2-bis(10,12-tricosadiynoyl)-sn-glycero-3-phosphoethanolamine (diyne PE), or any combination of the foregoing. The thiol-functionalized molecule can be a cell adhesion molecule. The cell adhesion molecule can be cyclic arginine-glycine-aspartic acid (cRGD).

Also provided is a further functionalized polymeric material on the surface of which is a patterned layer of polymerized amphiphiles, each of which comprises a functional head group (e.g., a chemically or biologically reactive or interactive head group) covalently bonded to an alkyl chain comprising a polymerizable diacetylene group, which polymerizes with another diacetylene group of an adjacent amphiphile to form a polydiacetylene, by which the amphiphiles are polymerized, wherein the alkyl chains are positioned to form a pattern of the functional head groups (e.g., chemically or biologically reactive or interactive head groups) about 1 nm in width at a periodicity from about 5 nm to about 10 nm, and wherein the polydiacetylenes are covalently bonded to a thiol-functionalized molecule. The pattern can be parallel lines. The periodicity can be about 5 nm to about 7 nm. The polymeric material can have a pore diameter from about 1 nm to about 200 nm. The polymeric material can be polyacrylamide. The amphiphile can be 10,12-TCD-NH₂, 10,12-PCD-NH₂, 10,12-TCD-OH, 10,12-PCD-OH, 10,12-TCD-COOH, 10,12-PCD-COOH, 4,6-TCD-NH₂, 4,6-PCD-NH₂, 4,6-TCD-OH, 4,6-PCD-OH, 4,6-TCD-COOH, 4,6-PCD-COOH, diyne PC, diyne PE, or any combination of the foregoing. The thio-functionalized molecule can be a cell adhesion molecule. The cell adhesion molecule can be cRGD.

A cell culture support comprising an above-described polymeric material is also provided. The amphiphile can be 10,12-TCD-NH₂, 10,12-PCD-NH₂, 10,12-TCD-OH, 10,12-PCD-OH, 10,12-TCD-COOH, 10,12-PCD-COOH, 4,6-TCD-NH₂, 4,6-PCD-NH₂, 4,6-TCD-OH, 4,6-PCD-OH, 4,6-TCD-COOH, 4,6-PCD-COOH, diyne PC, diyne PE, or any combination of the foregoing. The thiol-functionalized molecule can be a cell adhesion molecule. The cell adhesion molecule can be cRGD. amphiphiles are polymerized, wherein the alkyl chains are positioned to form a pattern of the functional head groups (e.g., chemically or biologically reactive or interactive head groups) about 1 nm in width at a periodicity from about 5 nm to about 10 nm, and wherein the polydiacetylenes are covalently bonded to a thiol of a thiol-functionalized molecule, under conditions that promote adhesion of the cells with the polymeric material. The pattern can be parallel lines. The periodicity can be about 5 nm to about 7 nm. The polymeric material can have a pore diameter from about 1 nm to about 200 nm. The polymeric material can be polyacrylamide. The amphiphile can be 10,12-TCD-NH2, 10,12-PCD-NH2, 10,12-TCD-OH, 10,12-PCD-OH, 10,12-TCD-COOH, 10,12-PCD-COOH, 4,6-TCD-NH2, 4,6-PCD-NH2, 4,6-TCD-OH, 4,6-PCD-OH, 4,6-TCD-COOH, 4,6-PCD-COOH, diyne PC, diyne PE, or any combination of the foregoing. The thiol-functionalized molecule can be a cell adhesion molecule. The cell adhesion molecule can be cRGD. The cells can be a mixture of cells. The cells can form a tissue.

### BRIEF DESCRIPTION OF THE FIGURES

The disclosed embodiments and other features, advantages, and aspects contained herein, and the matter of attaining them, will become apparent in light of the following detailed description of various exemplary embodiments of the present disclosure. Such detailed description will be better understood when taken into conjunction with the accompanying drawings.

Embodiments of the present disclosure will now be described by way of example in greater detail with reference to the attached Figures, in which:

FIG. 1A is a schematic of striped phase polydiacetylenes (sPDA) structure on a substrate 8 (e.g., highly ordered pyrolytic graphite (HOPG)) (top left); a schematic of functional pattern transfer of sPDAs from a substrate 8 to a scaffold 10 mesh (e.g., polydimethylsiloxane (PDMS) or polyacrylamide (PAAm)) (transfer layer identified as 12) (top right); and an illustration of PAAm compositional variables (% T and % C) that can impact transfer efficiency and the creation of 1-nm functional patterns with sub-10-nm pitch (bottom box).

FIG. 1B is a schematic illustrating the use of amine sPDAs on PAAm to control adsorption of double stranded deoxyribonucleic acid (dsDNA).

FIG. 1C is a schematic showing secondary functionalization of a surface 12 of a scaffold 10 via copper-free azidoalkane click reaction of the cell-adhesion peptide cyclic arginine-glycine-aspartic acid (cRGD) to carboxylic acid (—COOH) head groups on amphiphiles laid down on first a substrate (not shown) and thereafter—following transfer—on a scaffold 10 (e.g., PDMS or sPDA).

FIG. 1D is a schematic showing increased cell adhesion to polymeric material surface 12 functionalized with cRGD cell-adhesion peptides (functional head groups 50).

FIG. 1E is a schematic illustrating binding selectivity for wheat germ agglutinin (WGA) at striped interfaces based on choice of carbohydrate amphiphile used for striped phase assembly.

FIG. 1F is a schematic illustrating multivalent glycan recognition by WGA (PDB: 2UVO) showing four dimerized receptor sites with 1.3 nm spacing (top left); and a schematic of striped phase assembly and structure on HOPG (bottom left); a schematic of PDA transfer to PAAm in relation to PAAm polymerization architecture and extended glycopolymer display for selective multivalent binding (right, top and bottom).

FIG. 1G is a schematic illustrating the stochastic distribution of glycans in a typical standing-phase monolayer and showing the limited spatial and orientation control at 1-10 nm.

FIG. 2A are molecular models of unpolymerized and polymerized TCDGlcNAc monolayers on HOPG.

FIG. 2B is an atomic force microscopy (AFM) image (left) illustrating the lamellar and domain structures of a polymerized TCDGlcNAc monolayer, and a scanning electron microscopy (SEM) image illustrating the presence of microscale domain structure.

FIG. 2C is scanning electron microscopy (SEM) image illustrating the presence of microscopic vacancies.

FIG. 2D is fluorescence spectra of TCD-GlcNAc and TCD-GlcA on PAAm.

FIG. 2E are the chemical structures of GlcNAc and GlcA (left panels) and molecular models illustrating head group interactions in striped phases of TCD-GlcNAc and TCD-GlcA on HOPG (left, center panel).

FIG. 2F shows AFM micrographs of TCD-GlcNAc and TCD-GlcA on HOPG (right, center panel) and fluorescence micrographs of TCD-GlcNAc and TCD-GlcA on PAAm (right panel).

FIG. 3A are schematics illustrating transfer of sPDA on HOPG to PAAm by in situ crosslinking, where the top schematic shows carbohydrate head groups.

FIG. 3B is a fluorescence spectra image of 10,12-TCDGlcNAc and PAAm.

FIGS. 3C and 3D are fluorescence micrographs of TCD-GlcNAc/PAAm and PAAm, respectively.

FIGS. 4A-4D are fluorescence images of various monolayers after exposure to WGA (5 μg/mL), where FIG. 4A shows TCDGlcNA-functionalized PAAm, FIG. 4B shows TCDOH-functionalized PAAm, FIG. 4C shows tricosadiynamine (TCDA)-functionalized PAAm, and FIG. 4D shows TCDGlcA-functionalized PAAm.

FIGS. 4E-4H show fluorescence images of monolayer on PAAm.

FIG. 4I shows fluorescence spectra data of monolayers of FIGS. 4A-4D after exposure to WGA (5 μg/mL).

FIG. 4J shows fluorescence spectra of monolayers on PAAm.

FIG. 4K shows WGA binding to monolayers normalized for surface coverage.

FIG. 4L shows fluorescence spectra illustrating minimal increase in adsorption of rhodamine-labeled ConA (5 μg/mL) to TCD-GlcNAc/PAAm versus unfunctionalized PAAm. WGA exposed to unfunctionalized PAAm is shown for comparison as well. Higher gain settings were used in FIG. 4L to adequately resolve spectral differences at low emission intensity, leading to the higher observed fluorescence for the TCD-GlcNAc+WGA trace in comparison with the intensity in FIG. 4I.

FIGS. 4M-4O show μCP patterned TCD-GlcNAc (FIG. 4M) on HOPG, FIG. 4N shows ater transfer to PAAm, and FIG. 4O shows after transfer to PAAm and exposure to WGA. Inset in FIG. 4N uses enhanced contrast to emphasize the square pattern.

FIG. 5A shows molecular models of head group regions in polymerized monolayers of 10,12-TCD-GlcNAc/HOPG (top panel) and 4,6-TCD-GlcNAc/HOPG (bottom panel), illustrating the differences in head group organization between molecules.

FIG. 5B shows models following 4 ns dynamics with HOPG removed from PDA frozen, to simulate conditions on sPDAs transferred to PAAm.

FIG. 5C shows fluorescence intensities of 4,6-TCD-GlcNAc/PAAm and 10,12-TCD-GlcNAc/PAAm surfaces allowed to equilibrate with specified concentrations of WGA (0.5, 10, or 15 μg/mL).

FIG. 5D shows fluorescence intensity spectra data of 10,12-TCD-GlcNAc/PAAm and 4,6-TCD-GlcNAc/PAAm surfaces, before (top graph) and after (bottom graph) exposure to WGA (0.5 μg/mL).

FIG. 5E shows data related to the distribution of neighboring GlcNAc-GlcNAc distances along each sPDA. The gray tie line at 13.0 Å represents the distance between dimerized WGA binding sites.

FIG. 6A shows the molecular structure of TCD-NH₂ displayed above molecular models of unpolymerized and polymerized TCD-NH₂ on HOPG (TCD-NH₂/HOPG or TCD-NH₂@HOPG).

FIG. 6B is an AFM image of polymerized TCD-NH₂/HOPG showing lamellar periodicity.

FIGS. 6C and 6D are SEM images of microscale TCD-NH₂/HOPG structure.

FIG. 6E is an AFM micrograph image illustrating nanoscale lamellar ordering in striped domains of TCD-NH₂/HOPG.

FIG. 6F are SEM images of TCD-NH₂ illustrating microscale domain structure.

FIG. 7A shows a schematic of an experimental setup for sPDA transfer.

FIG. 7B shows fluorescence images of LS-TCD-NH₂ (left) and CP-TCD-NH₂/PAAm (right).

FIG. 7C shows fluorescence spectra of sPDA-functionalized PAAm and polydimethylsiloxane (PDMS).

FIG. 7D shows fluorescence spectra of μCP-TCD-NH₂/PAAm.

FIGS. 8A and 8B show fluorescence emission intensity of PAAm gels with varied % T (mass % of total acrylate in gel) (FIG. 8A) and varied % C (mass % of crosslinker vs. total acrylate) (FIG. 8B).

FIG. 8C shows a graph of PAAm pore size diameters calculated from cryogenic electronic microscopy (cryoEM) image analysis.

FIGS. 8D-8F show cryoEM images of pore structure and texture of an unfunctionalized PAAm gel with 10% T and 5% C (FIG. 8D), 30% T and 5% C (FIG. 8E), and 40% T and 5% C (FIG. 8F).

FIGS. 8G-8I show cryoEM images of pore structure and texture of TCD-NH₂/PAAm gel with 10% T and 5% C (FIG. 8G), 30% T and 5% C (FIG. 8H), and 40% T and 5% C (FIG. 8I).

FIG. 9A is a schematic illustrating the probability of transfer in relationship to local PAAm network density.

FIG. 9B are fluorescence images of TCD-NH₂/PAAm and PAAm gels with histograms of pixels at each hue, illustrating shift with % T.

FIG. 9C is an image of PAAm prepared at 50% T with increased contrast, showing faint speckling.

FIG. 9D illustrates the relationship between heterogeneous PAAm network structure and variability in crosslinking to PDA.

FIG. 10A shows the structures of polymerizable amphiphiles tested for DNA binding on sPDA/PAAm surfaces.

FIG. 10B shows a molecular model illustrating the relative scales of dsDNA and surface-patterned amines in sPDA.

FIG. 10C shows fluorescence spectra of TCD-NH₂/PAAm in the absence (green) and presence (red) of ethidium bromide-labeled DNA (EtBr-DNA).

FIG. 10D shows fluorescence emission of PAAm in the presence (red) and absence (gray) of EtBr-DNA.

FIG. 10E shows fluorescence spectra of TCD-NH₂/PAAm in the presence and absence of EtBr-DNA.

FIG. 10F shows composite fluorescence spectra from sPDA/PAAm substrates with amine, carboxylic acid, or hydroxyl surface patterns in the presence of EtBr-DNA.

FIG. 10G shows spectra from FIG. 10F with subtraction of bare PAAm in the presence of EtBr-DNA.

FIG. 11A is a molecular model of unpolymerized pentacosadiynamine (PCD-NH₂) on HOPG and polymerized and FIG. 11B is a molecular model of polymerized PCD-NH₂ on HOPG.

FIG. 11C is an AFM image of unpolymerized PCD-NH₂ on HOPG.

FIG. 11D is an AFM image of polymerized PCD-NH₂ on HOPG showing lamellar periodicity.

FIG. 12A shows fluorescence emission spectra of sPDA/PAAm before and after incubation with the functional azide arginine-glycine-aspartic acid (RGD).

FIG. 12B shows fluorescence emission spectra of sPDA/PAAm before and after incubation with the functional azide Bn-N₃ (Bn-N₃) with PAAm background subtraction.

FIG. 12C shows fluorescence mission spectra of sPDA/PAAm before and after incubation with the functional azide Bn-N₃ with PAAm background subtraction.

FIG. 12D shows fluorescence emission spectra of diyne phophoethanolamine (dPE) sPDA/PAAm before and after incubation with the functional azide Bn-N₃.

FIG. 13A is an optical image of C2C12 murine myoblasts cultured on bare PAAm.

FIG. 13B is an optical image of C2C12 murine myoblasts cultured on TCDA/PAAm.

FIG. 13C is an optical image of C2C12 murine myoblasts cultured on bare PAAm exposed to cRGD under click conditions.

FIG. 13D is an optical image of C2C12 murine myoblasts cultured on TCDA/PAAm+cRGD.

While the present disclosure is susceptible to various modifications and alternative forms, exemplary embodiments thereof are shown by way of example in the drawings and are herein described in detail.

### DETAILED DESCRIPTION

Controlling the chemistry of hydrogels and other substrates is important in many applications. In particular, being able to achieve local changes in substrate surface chemistry can provide benefits across a range of applications including the generation of gradient gels to improve chromatograph separation, the ability to microscale chemical environments in which local chemistry and mechanical properties can impact interactions with cells, and/or providing a dense, complex array of ligands that are expressed on cell surfaces to facilitate tissue regeneration, drug delivery, and the like.

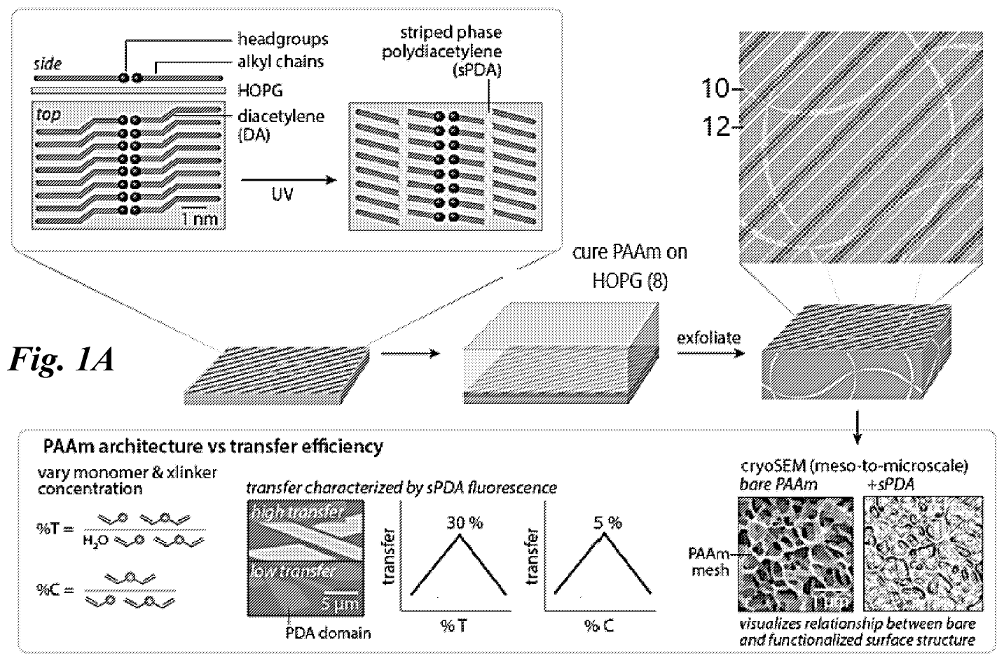

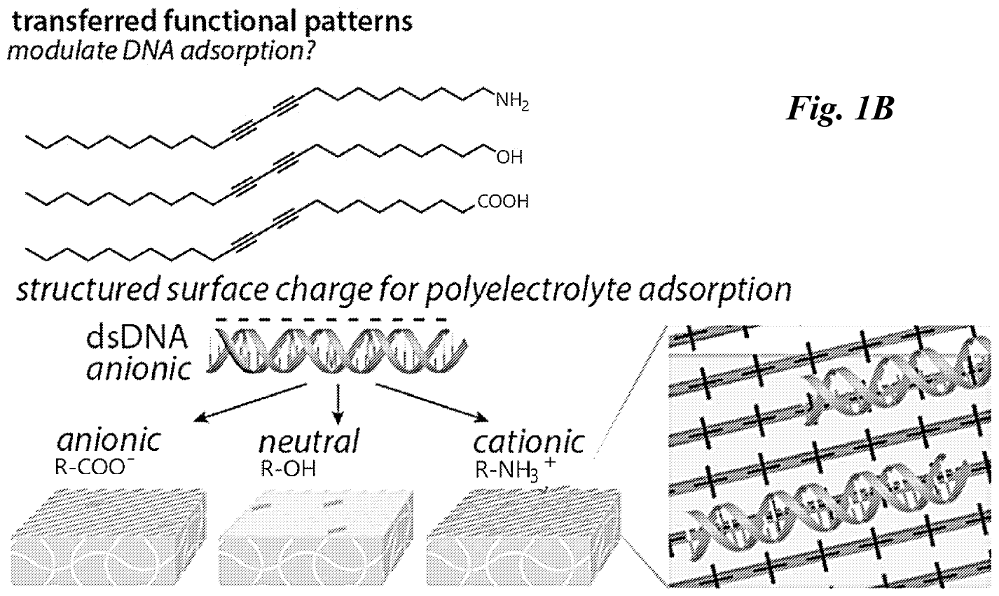

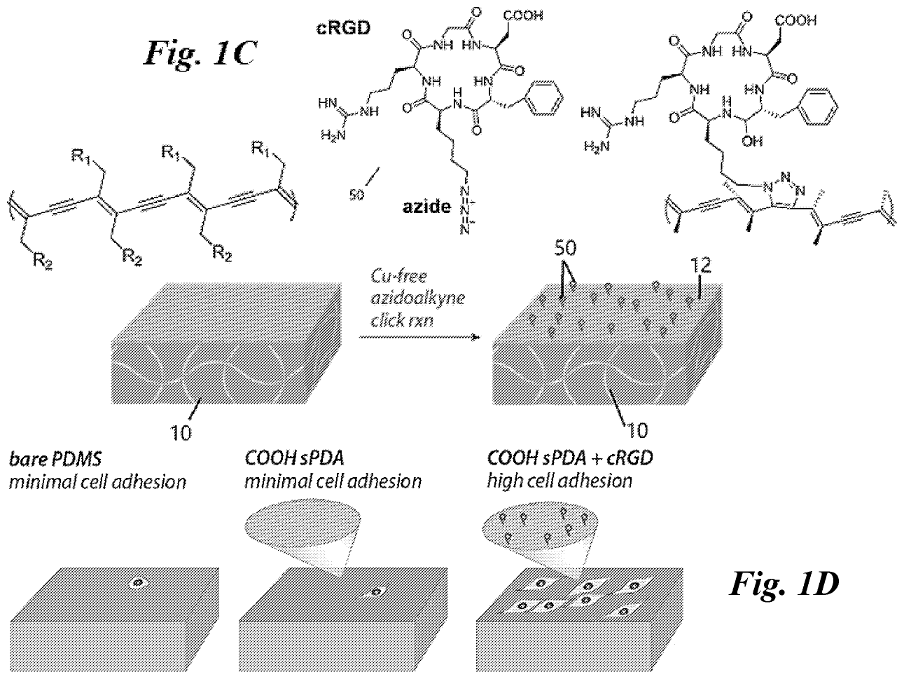

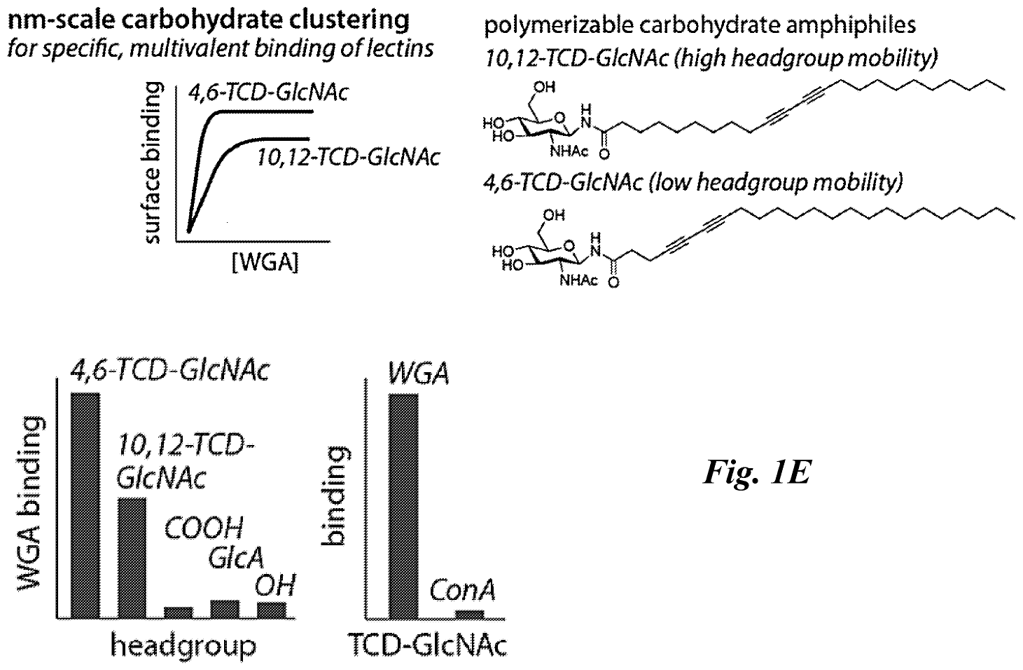

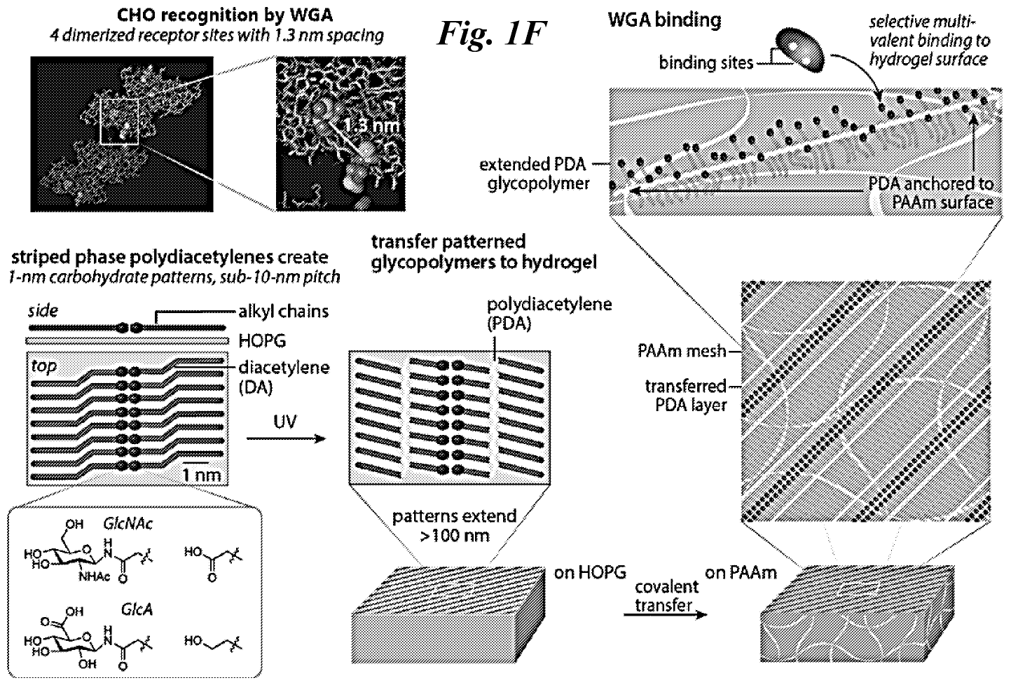

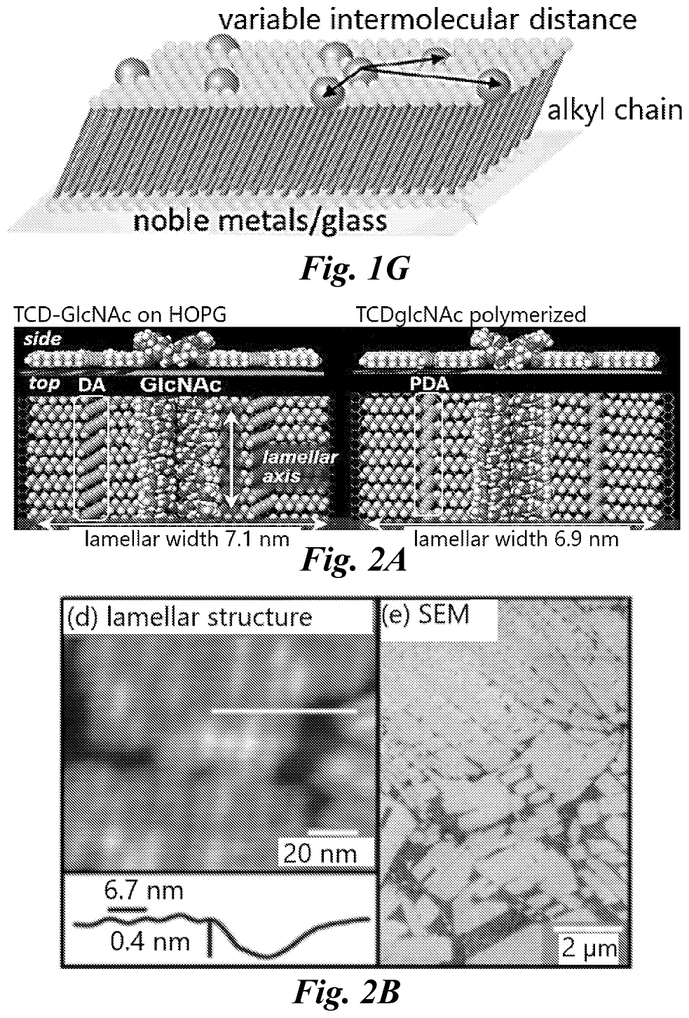

The present disclosure is predicated, at least in part, on a method of controlling the surface chemistry of a support material (e.g., a polymeric substrate such as, for example a hydrogel (e.g., a polyacrylamide (PAAm))) (FIG. 1B). The method involves a polymerization process involving in-situ radical reaction of a small molecule monomer (i.e., an amphiphile) to generate an ordered polymer network that displays one or more ligands in a repeating striped pattern. When, for example, covalently linked linear patterns of amines are transferred to the surface, they can also be used to control adsorption of deoxyribonucleic acid (DNA) at the interface, laying the groundwork for increasingly complex structure assembly.

The present methods provide detailed control over placement and orientation of ligands and/or functional head groups (such as carbohydrates, for example) on a surface of a polymeric substrate over scales from 1 to 10 nm or even up to 100 nm. As used herein, the term “ligand” is a molecule, ion, or atom that is attached to the central atom or ion. Geometrically localized patterns can also be generated by the local application of molecules to the surface using microcontact printing.

### Nanostructured Molecular Layers and Scaffolds

Novel molecular layers and scaffolds comprising such molecular layers are provided. A scaffold can comprise a molecular layer positioned on a surface of a support material. The support material can be an amorphous and/or soft substrate. The term “amorphous” means that the material does not exhibit diffraction Bragg peaks in the x-ray diffraction pattern that otherwise results from a repeating, long range periodic atomic or molecular ordering. The term “soft substrate” means any material that is flexible, pliable, or malleable when exposed to external force. Other physical characteristics common to soft substrates suitable for use in the scaffolds hereof include linear elasticity and incompressibility. Generally, soft substrates can have a Young's modulus in the range of about 1 to about 100,000 pascal (Pa). The soft substrates hereof can include those that can be tunable to a stiffness of physiological tissues with a Young's modulus of about 1,000 to about 100,000 Pa, about 1,000 to about 5,000 Pa, about 1,000 to about 10,000 Pa, about 5,000 to 10,000 Pa, about 5,000 to about 20,000 Pa, about 10,000 to about 20,000 Pa, about 10,000 to about 50,000 Pa, about 20,000 to about 50,000 Pa, about 30,000 to about 50,000 Pa, about 40,000 to about 50,000 Pa, about 50,000 to about 100,000 Pa, about 60,000 to about 100,000 Pa, about 70,000 to about 100,000 Pa, about 80,000 to about 100,000 Pa, about 90,000 to about 100,000 Pa. In certain embodiments, the soft substrate is prepared from a single material. In other embodiments, the soft substrate is prepared from more than one material. In yet other embodiments, a soft substrate is prepared by layering one or more soft substrates, e.g., to mimic tissue layers in vivo. The support material can be an amorphous, soft, and/or porous substrate.

In certain embodiments, a molecular layer comprises striped phase polydiacetylenes (sPDAs) with ordered patterns of functional groups thereon in nanometer resolution. sPDAs are a type of conjugated polymer that exhibit unique phase behavior, characterized by alternating regions of different molecular conformations. These regions can be aligned in stripes (or domains) and the properties of sPDAs are highly dependent on their molecular structure and organization.

The molecular layers hereof are customizable and are readily transferrable to a support material to functionalize the surface thereof—even an amorphous support material or soft substrate comprising substantial heterogeneity on length scales (e.g., a hydrogel).

The molecular layers and scaffolds hereof can pattern interactions with DNA, for example; a capability of potential importance for preconcentration in chromatographic applications, as well as for the development of nanostructured hybrid materials and supports for cell culture. Further, such molecular layers and scaffolds can comprise nanometer-resolution carbohydrate patterns to allow for selective, multivalent interactions (K_{D}˜40 nM) that can, for example, be used to design synthetic glycan-mimetic interfaces with features from molecular to mesoscopic scales, including soft scaffolds for cells.

Still further, the present disclosure is predicated, at least in part, on the surprising and unexpected discovery that sPDAs can participate in copper-free azidoalkyne “click” reactions, which typically require a strained alkyne (e.g., cyclooctyne) reaction partner. Accordingly, in certain embodiments, the monolayer can be further functionalized with secondary surface chemistry (e.g., secondary functionalities) installed along the sPDA backbone. In addition, the molecular layers can be secondarily functionalized with peptides and the like, making it possible to achieve a high degree of complexity with respect to the interface.

Conventionally, nanometer-resolution functional patterns have been difficult to achieve in broadly used interface chemistries such as functional alkyl-thiol monolayers on gold or functional alkylsilanes on silicon. However, 1-nm resolution functional patterns can be readily assembled on substrates such as highly oriented pyrolytic graphite (HOPG) using lying-down phases of simple diyne amphiphiles. Groszek, Preferential adsorption of long-chain normal paraffins on MoS₂, WS₂, and graphite from N-heptane, Nature 204: 680 (1964); Rabe & Buchholz, Commensurability and mobility in 2-dimensional molecular patterns on graphite, Science 253: 424-427 (1991); Okawa et al., Controlled chain polymerization and chemical soldering for single-molecule electronics, Nanoscale 4: 3013-3028 (2012); Claridge, Standing, lying, and sitting: translating building principles of the cell membrane to synthetic 2D material interfaces, Chemical Communications 54: 6681-6691 (2018).

Due to relatively strong noncovalent alkyl-H interactions, amphiphiles spontaneously assemble in a lying-down (or striped) orientation with the alkyl chains parallel to the substrate (e.g., HOPG) surface (see FIG. 1A) and are stabilized by the van der Walls interaction between adjacent alkyl chains and hydrogen-bonding between polar head groups. This can generate a polymer backbone on the initial substrate comprising, for example, striped 1 nm-resolution functional patterns with a specific pitch (e.g., sub-10 nm pitch). For amphiphiles with an internal diacetylene (DA), topochemical photopolymerization can be used to generate sPDAs (i.e. stripes of conjugated polydiacetylenes) that link together molecules (i.e., functional head groups) along the row or polymer backbone, which can be relatively rigid. Okawa & Aono, Linear chain polymerization initiated by a scanning tunneling microscope tip at designated positions, J Chemical Physics 115: 2317-2322 (2001). Amphiphiles assembled in this way can create 1-nm wide stripes of polar head groups with a pitch (e.g., 5-10 nm), tunable based on alkyl chain length. Shi & Claridge, Lipids: an atomic toolkit for the endless frontier, ACS Nano DOI: 10.1021/acsnano.1c07428 (2021). For example, in some cases, the periodicity equates with about twice the molecular length of the alkyl chain. Accordingly, lengthening or shortening the alkyl chains can have a direct effect on at least the periodicity of the pattern.

This type of patterning can be controlled over large areas and can influence both interfacial wetting and further assembly at the interface. Bang et al., Sitting phases of polymerizable amphiphiles for controlled functionalization of layered materials, J Am Chemical Soc'y 138: 4448-4457 (2016); Villarreal et al., Modulating wettability of layered materials by controlling ligand polar head group dynamics, J Am Chemical Soc'y 139: 11973-11979 (2017); Choong et al., Sitting phase monolayers of polymerizable phospholipids create dimensional, molecular-scale wetting control for scalable solution based on patterning of layered materials, ACS Applied Materials & Interfaces 9: 19326-19334 (2017).

Polydiacetylenes can also be utilized in a reaction process that transfers the nm-resolution functional patterns to soft, biocompatible scaffolds such as PDMS and polyacrylamide hydrogels. Davis et al. (2021), supra; Bechtold et al., Striped poly(diacetylene) monolayers control adsorption of polyelectrolytes and proteins on 2D materials and elastomers, ACS Applied Nano Materials 4: 7037-7046 (2021); Shi et al., Plenty of room at the top: a multi-scale understanding of nm-resolution polymer patterning on 2D materials, Angew Chemistry International Ed 60: 25436-25444 (2021).

Building on this, the present disclosure synthesizes polymerizable amphiphiles with various head groups using sPDA assembly to achieve nanometer-resolution patterning on a hard, crystalline material (e.g., the first substrate), and subsequently transferring the polymerized, patterned surface to a desired support material (e.g., a hydrogel or soft substrate) to form the scaffold.

The molecular layer is the portion comprising the functionalization that is formed on a first substrate and for placement on a support material to form the scaffold. In certain embodiments, a molecular layer (e.g., for placement on a support material) is provided. The molecular layer can be a monolayer. The terms “monolayer” and “thin film” can be used interchangeably herein without distinctions and are defined as a layer of material ranging from less than about 1 nm to several thousand nanometers in thickness. In some embodiments, a monolayer is a two-dimensional (2D) material, generally having a layer thickness between one atomic layer and a few nanometers (nm). In some embodiments, a monolayer is a three-dimensional (3D) material. In certain embodiments, a scaffold is provided comprising the molecular layer positioned on a surface of a support material. The molecular layer can be, in some embodiments, covalently bonded to the surface of the support material (e.g., using the methods hereof).

The support material can comprise any structure upon which a functionalized surface comprising the molecular layer is desired. The support material can be an amorphous material. The support material can comprise a soft substrate. The support material can comprise an amorphous and/or soft substrate. In certain embodiments, the support material is an amorphous, porous substrate.

In certain embodiments, the support material comprises a hydrogel. The hydrogel can be homopolymeric, copolymeric, or a multipolymer interpenetrating polymeric hydrogel (IPN), which is made of two independent cross-linked synthetic and/or natural polymer components contained in a network form. The hydrogel can be semicrystalline or crystalline. The hydrogel can be a matrix, a film or a microsphere. The hydrogel can be nonionic, ionic (e.g., anionic or cationic), ampholytic (i.e., containing both acidic and basic groups), or zwitterionic (i.e., containing anionic and cationic groups in each structural repeating unit).

The hydrogel can be a hydrophilic hydrogel. The hydrogel can comprise a polyacrylate, polyacrylamide, or polyethylene glycol (PEG) hydrogel. The hydrogel can be any type of hydrogel suitable for use as a cell adhesion scaffold. The hydrogel can be any type of hydrogel suitable for use as an implant. The hydrogel can be any type of hydrogel suitable for use as a cell support scaffold. In certain embodiments, the support material is compatible with a traditional cell culture support (e.g., such that the support material can be used to facilitate cell growth or adhesion).

The hydrogel can have any suitable pore diameter, such as from about 1 nm to about 200 nm. Typically, most hydrogels have a heterogeneous structure with pores of various sizes. For example, a hydrogel can have both small pores (e.g., about 1 to about 10 nm) and large pores (e.g., about 20 to about 200 nm).

In certain embodiments, the hydrogel comprises PAAm. PAAm is a hydrogel that is widely used in protein and deoxyribonucleic acid (DNA) chromatography, cell culture, and other applications. Nichols et al. (1985-1986), supra; Pandey & Mann, Proteomics to study genes and genomes, Nature 405: 837-846 (2000); Engler et al., Matrix elasticity directs stem cell lineage specification, Cell 126: 677-689 (2006). Gel formation is typically achieved through radical-mediated polymerization of an acrylamide (Aam) monomer with a bis-acrylamide (Bis) crosslinker. Pore sizes, and thus mechanical properties, can be controlled by adjusting the ratio of acrylate to water (% T), and the ratio of crosslinker to monomer (% C) as shown in FIG. 1A. The molecular layer can comprise polymerized amphiphiles organized in a lying-down orientation to form an exposed polymer backbone. “Amphiphile” means a chemical compound comprising hydrophilic and hydrophobic constituents. Each of the polymerized amphiphiles can comprise one or more alkyl chains. Additionally, each of the polymerized amphiphiles can comprise one or more functional head groups covalently bonded with an alkyl chain. The one or more functional head groups can be in an orientation capable of binding a target. Furthermore, the alkyl chains and functional head groups can be organized in a repeating striped pattern (e.g., comprising a width at a periodicity). In certain embodiments, the repeating striped pattern comprises lines substantially parallel to each other.

As previously described, due to relatively strong noncovalent alkyl-H interactions, the amphiphiles can spontaneously assemble in this lying-down (or striped) orientation and are stabilized by the van der Walls interaction between adjacent alkyl chains and hydrogen-bonding between polar head groups to generate the polymer backbone. The polymer backbone can orient in, for example, 1 nm-resolution functional patterns with a specific pitch (e.g., sub-10 nm pitch). In certain embodiments, the polymerized amphiphiles are organized in a lying-down (striped pattern) orientation that is substantially parallel to the surface of the support material, whereas the one or more exposed alkyl chains and the one or more functional head groups are not so restricted.

The amphiphile of the molecular layer can be any amphiphile suitable for inclusion in the molecular layer. In certain embodiments, the polymer backbone comprises a single type of amphiphile. In certain embodiments, the polymer backbone comprises two or more different amphiphiles. It will be appreciated that the types of amphiphile(s) used can be customized for the intended purpose of the resulting monolayer.

In certain embodiments, the amphiphile comprises a C₅-C₃₀ functionalized amphiphile with an internal diyne. The amphiphile can comprise a functionalized amphiphile with an internal diyne that comprises more than 30 carbons. The amphiphile can be or comprise 10,12-tricosadiynamine (10,12-TCD-NH₂). The amphiphile be or can comprise 4,6-tricosadiynamine (4,6-TCD-NH₂). The amphiphile can be or comprise 4,6-pentacosadiynamine (4,6-PCD-NH₂) or 10,12-pentacosadiynamine (10,12-PCD-NH₂). In certain embodiments, the amphiphile is or comprises 10,12-tricosadiynol (10,12-TCD-OH), 10,12-pentacosadiynol (10,12-PCD-OH), 10,12-tricosadiynoic acid (10,12-TCD-COOH), and/or 10,12-pentacosadiynoic acid (10,12-PCD-COOH). The amphiphile can be or comprise 1,2-bis(10,12-tricosadiynoyl)-sn-glycero-3-phosphocholine (diyne PC) or 1,2-bis(10,12-tricosadiynoyl)-sn-glycero-3-phosphoethanolamine (diyne PE).

In certain embodiments, the amphiphiles comprises a C₅-C₃₀ amine-functionalized amphiphiles with an internal diyne, such as, by way of non-limiting example, 10,12-TCD-NH₂ or 10,12-PCD-NH₂. In certain embodiments, the amphiphiles are or comprise 10,12-TCD-OH, 10,12-PCD-OH, 10,12-TCD-COOH, 10,12-PCD-COOH, 4,6-TCD-NH₂, 4,6-PCD-NH₂, 4,6-TCD-OH, 4,6-PCD-OH, 4,6-TCD-COOH, 4,6-PCD-COOH, diyne PC, diyne PE, or any combination of the foregoing.

Each amphiphile can comprise one or more alkyl chains. The one or more functional groups of the amphiphiles can be covalently bonded to the relevant alkyl chain. The alkyl chains can be exposed. The functional head groups can be exposed. The polymer backbone can be exposed (e.g., for attachment). These exposures result, at least in part, from the lying-down orientation of the polymer backbone, with the amphiphiles positioned parallel to the interface (i.e., lying flat) and can be important for transfer (i.e., the ability for the polymer backbone to undergo additional functionalization reactions). The polymer backbone can comprise polydiacetylene.

Prior to polymerization, each amphiphile can comprise a polymerizable functional group within the one or more alkyl chains. The amphiphiles can be polymerized through the polymerizable functional groups within the one or more alkyl chains.

The polymerizable functional groups (e.g., DA groups) can be positioned at any suitable position along the length of the alkyl chains. The position of the polymerizable functional group on an alkyl chain can affect accessibility of the chemically or biologically reactive head group (i.e., functional head group) as described in U.S. Pat. No. 10,889,669, which is hereby incorporated by reference for its teachings regarding the same.

The polymerizable functional groups can be polymerized by any suitable polymerization method. Examples of suitable polymerization methods are exposure to UV light (see, e.g., U.S. Pat. No. 10,525,502), heat, and/or energetic electrons. A device that can be used for in situ thermal control and transfer of the layer of polymerized amphiphiles onto the first substrate is described in U.S. Pat. No. 11,031,268, which is incorporated herein by reference for its teachings regarding the same. See also U.S. Patent Application Publication No. 2021/0060604 for the use of an apparatus to perform noncovalent functionalization of a substrate using Langmuir-Schaefer conversion (U.S. Patent Application Publication No. 2021/0060604 is hereby incorporated by reference for its teachings regarding the same).

For example, a polymerizable functional group can be a polymerizable DA group. For amphiphiles with an internal DA, polymerization can be used to generate sPDAs (i.e. stripes of conjugated polydiacetylenes) that link together molecules (i.e., functional head groups) along the row or polymer backbone.

The alkyl chains can be utilized to control the length between the polymer backbone and the patterned functionality (i.e., functional head groups). Accordingly, selection of the alkyl segments can be used to further tune the patterned design and properties of the interface. Pursuant to the present disclosure, such patterns can then be transferred to a scaffold (such as a hydrogel) surface, even when such scaffolds are soft and/or amorphous.

The alkyl chains can be positioned such that the functional head groups coupled thereto are organized in a pattern comprising a width at a periodicity (e.g., a repeating striped pattern dictated by the underlying pattern of the polymer backbone). The pattern can also comprise the functional head groups being positioned in an orientation capable of binding a target (e.g., a target at the interface such as a cell). These ordered molecular patterns can be polymerized (e.g., photopolymerized by ultraviolet (UV) irradiation) to “lock in” the pattern and generate striped arrays of PDAs with polymer backbones exposed at the interface for further reactions. By controlling the monomer structure and assembly conditions, it is possible to generate nanometer-resolution functional patterns on a substrate such as HOPG that extend over large microscopic areas and include diverse pattern symmetries and head group chemistries that impact macroscopic and nanoscopic wetting and assembly of nano- and microscopic objects at the interface.

These molecular layers comprising functional patterns can then be transferred to a support material as previously described.

The one or more alkyl chains of each amphiphile can be any suitable alkyl chains. “Alkyl” generally refers to a straight or branched hydrocarbon chain radical consisting solely of carbon and hydrogen atoms, such as having from one to fifteen carbon atoms (e.g., C₁-C₁₅ alkyl). Disclosures provided herein of an “alkyl” are intended to include independent recitations of a saturated “alkyl,” unless otherwise stated. An alkyl can comprise one to thirteen carbon atoms (e.g., C₁-C₁₃ alkyl). An alkyl can comprise one to eight carbon atoms (e.g., C₁-C₈ alkyl). An alkyl can comprise one to five carbon atoms (e.g., C₁-C₅ alkyl). An alkyl can comprise one to four carbon atoms (e.g., C₁-C₄ alkyl). An alkyl can comprise one to three carbon atoms (e.g., C₁-C₃ alkyl). An alkyl can comprise one to two carbon atoms (e.g., C₁-C₂ alkyl). An alkyl can comprise one carbon atom (e.g., C₁ alkyl). An alkyl can comprise about ten to thirty carbon atoms (e.g., C₁₀-C₃₀ alkyl). An alkyl can comprise about ten to about twenty-five carbon atoms (e.g., C₁₀-C₂₅ alkyl). An alkyl can comprise about fifteen to about twenty-five carbon atoms (e.g., C₁₅-C₂₅ alkyl). An alkyl can comprise about twenty to about twenty-five carbon atoms (e.g., C₂₀-C₂₅ alkyl). An alkyl can comprise about twenty-three to about twenty-five carbon atoms (e.g., C₂₃-C₂₅ alkyl). In other embodiments, the alkyl chain is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). An alkyl chain can be a nonpolar alkyl chain.

The alkyl chain can be attached to the rest of the molecule by a single bond. The alkyl chains can be linear or branched. Linear alkyl chains can be preferrable in certain applications.

The one or more functional head groups can be covalently bonded with an exposed alkyl chain. The functional head groups can comprise chemically or biologically reactive or interactive head groups. “Chemically or biologically reactive or interactive” is used herein to describe a functional head group on an amphiphile that can undergo a chemical or biological reaction or interaction with a component of a sample, such as a cell or a tissue, for example. The head group can be cationic under physiological conditions and can be neutral during deposition of the patterned layer or assembly.

The one or more functional head groups can be polar head groups.

A functional head group can be an amine, a CHO, a carboxylate, an alcohol, a peptide a substrate of an enzyme, a nucleic acid, a lipid, or any combination of the foregoing.

A functional head group can be a CHO head group. In certain embodiments, the CHO head group can be or comprise a monosaccharide, a glycan, a N-acetyl-D-glucosamine (GlcNAc), a glucuronic acid (GlcA), and/or a N-acetyl-D-neuraminic acid.

The functional head group can be neutral. In certain embodiments, the functional head group is neutral and comprises a hydroxyl. The head group can be a zwitterionic head group. In certain embodiments, the functional head group is a zwitterionic head group and comprises a phosphoethanolamine. The head group can be anionic under physiological conditions and can be neutral during deposition of the patterned layer or assembly. The anionic head group can be a carboxylate.

In certain embodiments, the head groups comprise CHO head groups (e.g., N-acetyl-D-glucosamine (GlcNAc)) to achieve nm-resolution CHO patterning that mimics key aspects of cell-surface-glycan architectures.

As noted above, the one or more functional head groups are organized in a pattern comprising a width at a periodicity and further in an orientation capable of binding a target (e.g., a cell, a biomolecule, a bioactive, and/or a myoblast). The repeating elements of the striped pattern can be linear and substantially parallel to each other. The pattern in which the functional head groups are organized can be any ordered pattern but such pattern is typically, at least in part, dictated by the organization of the polymer backbone. In certain embodiments, the pattern comprises parallel lines. In certain embodiments, the pattern of the one or more functional head groups is a nanoscale pattern (e.g., a pattern having a nanoscale). The pattern of the one or more functional head groups can have a sub-10-nm scale.

The pattern of the one or more functional head groups can be organized to mimic certain desired properties, for example, of nature. For example, the pattern of the one or more functional head groups (i.e., comprising CHO head groups) can mimic properties of polysaccharide components (e.g., hyaluronic acid). In certain embodiments, the CHO type can be selected, and the pattern of the one or more functional groups designed, such that, in use, the CHOs act together as a component of an extracellular matrix (ECM). In certain embodiments, the pattern of the one or more functional head groups mimics properties of macromolecules or components of an ECM (for example, as a means for modulating cell adhesion, spreading, proliferation or differentiation, or for cellular reprogramming). This is possible due to the detailed control over placement and orientation of CHOs provided by the monolayers hereof. The monolayer can be useful for modulating cell adhesion, cell proliferation, cell differentiation, and/or reprogramming of a cell. In such cases, one or more of the functional head groups can comprise a CHO. The placement and orientation of the CHOs can be selected over scales from 1 to 10 nm for individual lectins and up to 100 nm or more when lectins themselves are clustered, as in a viral capsid. For instance, the wheat germ agglutinin (WGA) protein exhibits binding specificity for GlcNAc and N-acetyl-D-neuraminic acid (sialic acid, Sia) with a 1.3 nm distance between receptor sites for GlcNAc (FIG. 1F) while concanavalin A (ConA) has a distance of about 6.5 nm between mannose binding sites. Carbohydrate linkage and ring orientation are also important factors (e.g., α2,3-linked Sia is associated with avian influenza viruses and α2,6-linkages with human viruses). Such interactions can also span larger scales when lectins are clustered for recognition. For example, the diameter of a typical hemagglutinin lectin expressed on the influenza A virus (IAV) surface is ca. 14 nm, while the virus diameter is 100-150 nm; the targeted glycan length is ca. 2-20 nm, with multiple glycans binding to lectins across the IAV surface. More broadly, the distribution of glycans across a cell surface can lead to pattern formation over length scales from nanometers to micrometers, extending to even larger scales across tissues.

The molecular layers hereof can be used to display monosaccharides and glycans at interfaces, providing convenient readouts of binding interactions, for example, as well as the potential for longer-range ensemble interactions. The molecular layers hereof can comprise a multi-valent binding surface.

The one or more functional head groups are held in an orientation capable of binding a target. The target can be a cell (e.g., a myoblast). The target can be a biomolecule. The target can be a bioactive molecule.

In certain embodiments, the width of the pattern of the functional head groups is at or about 1 nm (such as 1 nm). In certain embodiments, individual ordered regions of the polymerized amphiphiles have linear dimensions of greater than about 100 nm in length while maintaining nanoscale periodicity of the striped pattern of function head groups. The ordered regions of the polymerized amphiphiles can have linear dimensions of greater than about 10 μm in length (such as 10 μm, 11 μm, 12 μm, 15 μm, or greater than 15 μm) while maintaining nanoscale periodicity of the striped pattern of functional head groups. The ordered regions of the polymerized amphiphiles can have linear dimensions of greater than about 1 μm in length (such as 1 μm, 2 μm, 3 μm, or greater than 4 μm) while maintaining nanoscale periodicity of the striped pattern of functional head groups.

The periodicity can be any suitable periodicity, such as about 5 nm to about 7 nm (such as 5 nm to about 7 nm or about 5 nm to 7 nm) or about 5 nm to about 10 nm (such as 5 nm to about 10 nm or about 5 nm to 10 nm). The pattern of the one or more functional head groups can have a sub-100-nm scale.

The width of the pattern of functional head groups can be at or about 1 nm (such as 1 nm). In certain embodiments, the width of a striped phase is at or about 1 nm (such as 1 nm). In certain embodiments, the width of the pattern is about 1 nm to about 2 nm (such as about 1 nm to 2 nm, 1 nm to about 2 nm, or 1 nm to 2 nm).

The molecular layer hereof can be for placement on a support material, for example. In certain embodiments, the molecular layer is transferred to a surface of a support material. In certain embodiments, the molecular layer is covalently transferrable to the support material.

The polymer backbone (e.g., the exposed polymer backbone) of the molecular layer can optionally be secondarily functionalized. In certain embodiments, the polymer backbone of the molecular layer can participate in copper-free azidoalkyne “click” reactions. Using this chemistry, complex secondary surface functionalization moieties, such as cyclic arginine-glycine-aspartic acid (cRGD) cell-adhesion peptides or fragments or functional analogs thereof, can be applied to the surfaces of polymeric materials. Indeed, the molecular layer can provide a straightforward and structured display of a wide range of azide- or thiol-functionalized molecules on its surface, even where the support material is a soft, biocompatible material and in addition to any primarily functionalization afforded by the functional head groups.

Accordingly, the polymer backbone can be covalently bonded to an azide-functionalized or a thiol-functionalized molecule in the absence of copper. The azide-functionalized molecule or the thiol-functionalized molecule can be a cell adhesion molecule. In certain embodiments, the cell adhesion molecule is cRGD.

In certain embodiments, the functionality of the molecular layer comprises CHO, one or more peptides, a tripeptide sequence of RGD or a functional analog or fragment thereof, a matrisome component, or a combination of any of the foregoing.

The scaffolds and/or molecular layers can be for use in cell culturing, chromatographic biomolecule separation, modulating microscale chemical environments, as a regenerative medicament or implant, or any combination of the foregoing.

In certain embodiments, the scaffold comprises a molecular layer comprise diyne amphiphiles with CHO head groups assembled in nm-resolution CHO patterns covalently bonded to a surface of a hydrogel. In spite of the significant heterogeneity of the hydrogel, transferred patterns of appropriate CHOs (e.g., GlcNAc) can enable selective interactions with wheat germ agglutinin (WGA), a model lectin that exhibits multivalent binding with appropriately spaced GlcNAc moieties. Additionally, WGA binding selectivity can be modulated by the choice of alkyl chain structure, supporting that this strategy can be used to further control CHO presentation at the interface. More broadly, this approach has potential utility in the generation of glycan arrays and in designing interfaces for controlled interactions with cells.

The present scaffolds and/or molecular layers can also be used for multivalent receptor microarrays for screening molecular interactions, display geometries, spatial organization of ligands and/or other binding characteristics. In certain embodiments, the multivalent receptor microarray can comprise a lectin assay for screening glycan-lectin interactions. For example, the molecular layer can control glycan display from molecular to microscopic scales, on both hard and soft substrates, which combines desirable aspects of polymer and surface-based approaches to bridge length scales. In certain embodiments, such approach can be utilized as a potent screening and inhibitory tool for glycan binding proteins (GBPs).

Glycan microarrays have been the most broadly successful approached used to study glycan-lectin interactions; in these arrays, functionalized monosaccharides or glycans are covalently attached to derivatized solid surfaces such as glass, silicon, and gold. Hyun et al., The glycan microarray story from construction to applications, Accounts of Chemical Research 50: 1069-1078 (2017). However, to date, two significant challenges of this conventional approach remain: (1) limited control over glycan orientation and inter-glycan distances when glycan density is decreased, and (2) the hardness of the functionalized substrates, which limits capacity for screening interactions under biologically relevant mechanical conditions. Some studies have demonstrated control over glycan density through macromolecular scaffolds (e.g., polymers, dendrimers, clusters, peptides, and proteins); however, synthetic polymer scaffolds typically provide limited control over glycan spacing or orientation, thereby decreasing selectivity. Kiessling & Grim, Glycopolymer probes of signal transduction, Chem Soc'y Reviews 42: 4476-4491 (2013); Di Maio et al., Controlled density glycodendron microarrays for studying carbohydrate-lectin interactions, Organic & Biomolecular Chem 19: 7357-7362 (2021); Mueller et al., Organizing multivalency in carbohydrate recognition, Chem Soc'y Reviews 45: 3275-3302 (2016); Lin et al., Controlling ligand spacing on surface: polyproline-based fluorous microarray as a tool in spatial specificity analysis and inhibitor development for carbohydrate protein interactions, ACS Applied Materials & Interfaces 9: 41691-41699 (2017); Zhang et al., An array-based method to identify multivalent inhibitors, J Am Chemical Soc'y 132: 9653-9662 (2010); Fasting et al., Multivalency as a chemical organization and action principle, Angewandte Chemie-International Ed 51: 10472-10498 (2012). Conversely, peptide- and protein-based scaffolds that provide elements of control over spacing (e.g., cyclic polyprolines) require complex synthesis.

Developing a modular, biocompatible scaffold with precisely-controlled chemistries thereon can offer numerous advantages that are significant to various industries. Glycan density and orientation could lead to improved glycan screening array performance by more closely replicating multivalent binding geometries observed in vivo. Nanometer-resolution functional patterns are difficult to achieve in broadly used monolayer chemistries such as functional alkylthiol monolayers on gold or functional alkylsilanes on silicon. However, 1-nm resolution functional patterns can be readily assembled on substrates such as HOPG and, pursuant to the methods hereof, transferred to an amorphous, soft, and/or porous support material (e.g., a hydrogel or the like). In certain embodiments, a scaffold is provided that comprises a surface upon or to which a molecular layer is applied and/or covalently transferred. The scaffold can be a cell culture support. The scaffold can be a cell support material compatible to be added to a cell culture support. The scaffold can comprise a hydrogel. In certain embodiments, the cell culture platform can comprise an in vivo cell culture platform (i.e., designed for use in vivo). In certain embodiments, the cell culture platform can comprise an in vitro cell culture platform (i.e., designed for use in vitro). Non-limiting examples of an in vitro cell culture platform include a 3D cell culture platform, an organ-on-a-chip platform, an immune cell culture platform, and an induced pluripotent stem cell culture platform.

Receptors or multivalent lectin assay formats are also provided. In certain embodiments, the receptor or multivalent lectin assay format comprises any of the hydrogels described herein.

A chromatography or biosensing platform, a cell culture support, or an implant for regenerative medicine can comprise any of the scaffolds hereof, on the surface of which is a patterned layer of polymerized amphiphiles. When used as part of a chromatography platform, the clustering of functional groups on the material can be used to induce selective binding of an analyte for purification and/or the surface display can be used to generate customized chromatographic stationary phases displaying complex molecules needed for bio-separation or to create multimodal stationary phases. The functional head groups of the monolayer for such application can be head groups that bind DNA, for example.

When used as part of a cell culture support, the spatial arrangement and/or orientation and coupling of functional head groups (e.g., chemically or biologically reactive or interactive head groups) can mimic structural elements of macromolecular components (e.g., polysaccharides, such as hyaluronic acid) of an ECM, such as one encoded by a matrisome. The support can be used to culture any cell, such as one used to engineer a tissue or repair a tissue, e.g., a myoblast, in vitro or in vivo. n an embodiment of a cell culture support, the amphiphile can be TCD-NH₂, PCD-NH₂, TCD-OH, PCD-OH, TCD-COOH, PCD-COOH, diyne PC, diyne PE, or any combination of the foregoing. The thiol-functionalized molecule can be a cell adhesion molecule. The cell adhesion molecule can be cRGD.

Implants are also provided. The implant can comprise a molecular layer bonded (e.g., covalently bonded) to a surface of a support material such as a hydrogel. The implant can be an implantable biological device. The implant can be an implantable hardware. When used as part of an implant for regenerative medicine, for example, the surface can be used to display peptides or proteins such as those that are components of biological basement membranes. It will be understood that the monolayer and materials hereof can help to facilitate cell adhesion and/or growth in and around an implant displaying the monolayer and/or materials hereof.

### Methods

In view of the foregoing, a method of preparing a hydrogel comprising a functionalized surface is provided.

The method can comprise providing or obtaining a first substrate comprising a molecular layer positioned thereon. Any of the molecular layers, support materials, and/or scaffolds described herein can be used with this method. For example, the molecular layer can comprise polymerized amphiphiles organized in a lying-down orientation to form an exposed polymer backbone. Each of the polymerized amphiphiles can comprise one or more alkyl chains and one or more functional head groups covalently bonded with an alkyl chain. Further, the one or more functional head groups can be in an orientation capable of binding a target (e.g., a cell, bioactive molecule, biomolecule, or the like) and the alkyl chains and functional head groups can be organized in a repeating striped pattern comprising a width and periodicity.

In certain embodiments, the one or more alkyl chains lie flat on the first substrate (at least following polymerization) and are positioned such that the functional head groups are organized in the pattern. The pattern of the functional head groups can be, for example, any of the patterns described herein such as about 1 nm to about 2 nm in width (e.g., about 1 nm to 2 nm, 1 nm to about 2 nm, or 1 nm to 2 nm) at a periodicity from about 5 nm to about 10 nm (e.g., about 5 nm to 10 nm, 5 nm to about 10 nm, or 5 nm to 10 nm). The pattern of the functional head groups can be, for example, parallel lines. The one or more functional head groups can be chemically or biologically reactive or interactive head groups. The one or more functional head groups can be chemically or biologically reactive or interactive head groups which are cationic under physiological conditions. In certain embodiments, the cationic head group is an amine. In certain embodiments, the one or more functional head groups comprises CHO head groups. The functional head groups can be any of those described herein or would be reasonably known in the art in view of the present disclosure. The functional head groups can be neutral or zwitterionic under physiological conditions. The head groups can be a neutral head group such as hydroxyl. An example of a zwitterionic head group is phosphoethanolamine. The chemically or biologically reactive or interactive head groups can be anionic under physiological conditions. An example of anionic head groups is carboxylate.

The polymerizable functional groups can, as previously described, comprise DA groups. In such instances, polymerization of the DA groups with DA groups of adjacent amphiphiles can form polydiacetylene within the exposed polymer backbone.

The amphiphiles can comprise C₅-C₃₀ functionalized amphiphiles with an internal diyne. The amphiphiles the amphiphiles comprise TCD-NH₂, PCD-NH₂, or a combination of the foregoing. The amphiphiles can comprise 10,12-TCD-GlcNAc, 4,6-TCD-GlcNAc, TCD-OH, PCD-OH, TCD-COOH, PCD-COOH, diyne PC, diyne PE, or any combination of the foregoing.

While the first substrate comprising the molecular layer positioned thereon can be provided independently, it can optionally be generated. In certain embodiments, obtaining the first substrate comprising the molecular layer comprises: (1) ordering polymerizable amphiphiles as a standing phase film on an aqueous subphase, each of the polymerizable amphiphiles comprising one or more alkyl chains comprising a polymerizable functional group and one or more functional head groups covalently bonded with an alkyl chain; (2) contacting the first substrate to the film in a manner that reorders the polymerizable amphiphiles from the standing phase into ordered a repeating striped pattern on the first substrate, wherein the ordered pattern forms a polymer backbone in which the polymerizable amphiphiles are organized in a lying-down orientation and define the pattern of the one or more functional head groups; and (3) polymerizing the polymerizable functional groups of the polymerizable amphiphiles with polymerizable functional groups of adjacent amphiphiles to stabilize the pattern and polymerize the amphiphiles. In such cases, the alkyl chains can lie flat on the first substrate and can be positioned such that the functional head groups are organized in the pattern.

The patterned layer of polymerized amphiphiles affixed to the first substrate is then transferred to a surface of a hydrogel/support material by covalent bonding such that the surface of the cured hydrogel comprises the patterned layer of polymerized amphiphiles comprising the one or more functional head groups. Furthermore, the alkyl chains can be exposed following transfer of the molecular layer to the surface of the support material/hydrogel. Additionally, the polymer backbone and the head groups of the molecular layer are also exposed and available for binding/reactions. The pattern of the one or more functional head groups can be dictated by the organization of the polymerized amphiphiles within the exposed backbone.

During curing, which can involve a cross-linking reaction between a vinyl-terminated base polymer and a cross-linker containing reactive Si—H bonds, for example. Curing a scaffold such as a hydrogel in contact with the sPDA molecular layer hereof, then exfoliating, can result in transfer of the sPDA monolayer to the scaffold surface, thereby functionalizing the scaffold. This surface functionalization can be leveraged to enable control over interactions with nanoscale objects (e.g., inorganic nanowires) and molecules (e.g., polyelectrolytes or CHOs) in the environment.

The first substrate can be any suitable substrate. In certain embodiments, the substrate comprises graphene. The substrate can comprise polydiacetylene. The substrate can comprise PDMS. The substrate can comprise polyacrylamide (PAAm). In certain embodiments, the substrate comprises a layered material comprising MoS₂ or WS₂. The substrate can comprise pyrolytic graphite (HOPG). The first substrate can comprise polydimethylsiloxane (PDMS; also known as dimethylpolysiloxane or dimethicone).

The first substrate can have any suitable dimensions and can be 2D or 3D. The first substrate can be of any suitable thickness. Graphene, for example, can be less than 1 nm in thickness, whereas graphite can be about 0.1-2 mm in thickness.

The first substrate can comprise a multivalent binding surface (e.g., by virtue of the polymerized amphiphiles and one or more functional head groups thereof). In certain embodiments, the first substrate has a pore diameter from about 1 nm to about 200 nm (such as 1 nm to about 200 nm, about 1 nm to 200 nm, or 1 nm to 200 nm).

The first substrate can be prepared using any suitable method known in the art, such as Langmuir-Schaefer (see, e.g., U.S. Pat. No. 10,756,278 for an improved method).

Accordingly, the method comprises providing the first substrate comprising a patterned layer of polymerized amphiphiles thereon. Such patterned orientation of the polymerized amphiphiles can dictate the pattern of the one or more functional head groups.

To transfer the patterned layer to a surface of the curing hydrogel an aqueous reaction mixture comprising a monomer, a crosslinker, and a radical initiator can be applied on top of the patterned layer of polymerized amphiphiles. In such case, the aqueous reaction mixture can cure by free-radical polymerization to form the hydrogel while in contact with the patterned layer. In this manner, the patterned layer is transferred to a surface of the cured hydrogel.

The aqueous reaction mixture can be any suitable aqueous reaction mixture that can be polymerized into a hydrogel which, depending on the application, can desirably be biocompatible. Examples of aqueous reaction mixtures include, without limitation, those that result in the production of polyacrylamide, poly-2-hydroxyethyl methacrylate, polyacrylic acid, polyacrylonitrile, cross-linkable polyethylene glycol (PEG), protein (e.g., collagen or gelatin), and/or polysaccharides (e.g., starch, alginate, or agarose).

The monomer in the aqueous reaction mixture can be any monomer that can be polymerized into a hydrogel, which, depending on the application, can desirably be biocompatible. The aqueous reaction mixture can comprise a single type of monomer or a mixture of monomers. Likewise, each monomer can comprise one or more functional groups, such as acrylic acrylamide, allyl, PEG diacrylate, vinyl, methacrylic, alkenyl, and any combination thereof. Examples of monomers include, but are not limited to, an acrylamide monomer or a diacrylamide monomer (such as when the hydrogel is polyacrylamide), poly(2-hydroxyethylmethacrylate), methacrylic acid, N-isopropylacrylamide, collagen methacrylate, and gelatin methacrylate.

In certain embodiments, the reaction mixture comprises from about 10% to about 50% total acrylamide (e.g., 10% to about 50%, about 10% to 50%, or 10% to 50%) and from about 1% to about 10% bisacrylamide (e.g., 1% to about 10%, about 1% to 10%, or 1% to 10%), as a percentage of total acrylamide in each instance.

If desired, one or more degradation sites, which can be the same or different, can be incorporated into the monomer. The site can be selected to be degraded by hydrolysis, photolysis, microbes, or enzymes, for example. Examples of degradation sites include, but are not limited to, polylactic acid and matrix-metalloproteinase sensitive (MMP-sensitive) peptide.

The crosslinker in the aqueous reaction mixture can be any crosslinker that can polymerize the monomer. Examples of crosslinkers include, but are not limited to, bisacrylamide (such as when the monomer is an acrylamide or diacrylamide), PEG dimethacrylate, and N,N′-methylene bisacrylamide. In certain embodiments, the crosslinker is bisacrylamide, the radical initiator is ammonium persulfate, and the radical stabilizer is N,N,N′,N′-tetramethylethylenediamine.

The radical initiator in the aqueous reaction mixture can be any radical initiator that can initiate free radicals for polymerization of the monomer. Examples of radical initiators include, but are not limited to, ammonium persulfate (such as when the monomer is an acrylamide or diacrylamide), azoisobutylnitrile, benzoylperoxide, or benzoin.

The aqueous reaction mixture can further comprise a radical stabilizer. The radical stabilizer can be any radical stabilizer that can stabilize the free radicals for polymerization of the monomer. Examples of radical stabilizers include, but are not limited to, N,N,N′,N′-tetramethylethylenediamine (such as when the monomer is an acrylamide or diacrylamide).

The reaction mixture can comprise from about 10% to about 50% total acrylamide and from about 1% to about 10% bisacrylamide (as a percentage of total acrylamide), such as about 30% total acrylamide and about 5% bisacrylamide (as a percentage of total acrylamide). In other embodiments, the reaction mixture can comprise about 10% PEG diacrylate, with appropriate amounts of ammonium persulfate (APS) and N,N,N′,N′-tetramethylethylenediamine (TEMED) (such as about 0.1% by mass).

The aqueous reaction mixture is allowed to cure by an appropriate curing method depending on the components of the mixture. An example of a curing method is free-radical polymerization.

Transferring the molecular layer to the surface of a support material can be performed concurrently or sequentially with curing the support material/hydrogel. For example, an aqueous reaction mixture can be applied on top of the molecular layer which is positioned over and/or adjacent to an uncured hydrogel. The aqueous reaction mixture can comprise a monomer, a crosslinker, and a radical initiator such that the aqueous reaction can undergo free radical polymerization to form the hydrogel or otherwise crosslink and/or bond the molecular layer therewith.

The hydrogel can alternatively be cured prior to the transfer step. In such instances, a layer of crosslinker can be applied to the surface of the hydrogel to which the molecular layer is to be transferred such that such excess crosslinker facilitates the covalent bonding therewith. Additionally or alternatively, the hydrogel can be formulated to comprise available reaction sites on the targeted surface such that the molecular layer can covalently bond therewith.

In certain embodiments the hydrogel, to the surface of which has been transferred by covalent bonding the patterned layer of polymerized amphiphiles, is exfoliated from the first substrate by any suitable method known in the art. An example of an exfoliating method is gentle insertion of a sharp blade between the hydrogel and the first substrate at the edge of the first substrate. Exfoliation can be assisted through the introduction of water or other polar solvent that wets the hydrogel.

The hydrogel can be homopolymeric, copolymeric, or a multipolymer IPN, which is made of two independent cross-linked synthetic and/or natural polymer components contained in a network form. The hydrogel can be amorphous, semicrystalline, or crystalline. The hydrogel can be a matrix, a film or a microsphere. The hydrogel can be nonionic, ionic (e.g., anionic or cationic), ampholytic (i.e., containing both acidic and basic groups), or zwitterionic (i.e., containing anionic and cationic groups in each structural repeating unit).

The hydrogel can have any suitable pore diameter, such as from about 1 nm to about 200 nm. Typically, most hydrogels have a heterogeneous structure with pores of various sizes. For example, a hydrogel can have both small pores (e.g., about 1 to about 10 nm) and large pores (e.g., about 20 to about 200 nm).

Transfer of the patterned layer of polymerized amphiphiles can primarily occur in areas of the hydrogel with pore sizes below about 100 nm. For hydrogels made from collagen or gelatin, pore diameters may be larger, e.g., up to 5 μm.

After applying the aqueous reaction mixture on top of the patterned layer of polymerized amphiphiles, the method can further comprise applying a second substrate, which can be the same as or different from the first substrate, on top of the aqueous reaction mixture. The second substrate can comprise on its surface, which faces the aqueous reaction mixture, a layer of reactive functional groups, e.g., one or more alkenes, or a patterned layer of polymerized amphiphiles, which can be the same as or different from the patterned layer of polymerized amphiphiles of the first substrate. Non-limiting examples of reactive functional groups include, without limitation, a peptide (such as a peptide comprising the amino acid sequence Arg-Gly-Asp (RGD) or a fragment or functional analog thereof), a substrate of an enzyme, a nucleic acid, a lipid, and/or a component of an ECM (such as one encoded by a matrisome). In certain embodiments, the one or more functional head groups comprises a CHO, and the reactive functional group(s) comprises a tripeptide sequence of RGD (e.g., cyclic RGD) or a fragment or functional analog thereof, a matrisome component, or a combination of any of the foregoing.

After the aqueous reaction mixture is allowed to cure, the hydrogel can also be covalently bonded to the second substrate. An example of a different substrate is glass (e.g., vinyl-functionalized glass). In certain embodiments, the first and second substrates comprise the same material selected from the group consisting of HOPG, graphene, and a layered material comprising MoS₂ or WS₂.

Where secondary functionality is desirable on the molecular layer and/or patterned surface, the method can further comprise contacting the patterned layer of polymerized amphiphiles on the surface of the hydrogel with a thiol-functionalized molecule or an azide-functionalized molecule under conditions that promote a click reaction between a polydiacetylene and a thiol of the thiol-functionalized molecule or between a polydiacetylene and an azide of the azide-functionalized molecule. The thiol-functionalized or azide-functionalized molecule can be a cell adhesion molecule. In certain embodiments, the cell adhesion molecule is cRGD.

Methods are also provided for adsorbing a biomolecule in a sample. The method can comprise contacting the sample with a hydrogel hereof (e.g., a polyacrylamide hydrogel), on the surface of which is a patterned layer of polymerized amphiphiles comprising a functional head group under adsorptive conditions, whereupon the biomolecule in the sample is adsorbed onto the surface of the hydrogel. The biomolecule can be DNA, for example. The patterned layer of polymerized amphiphiles can be any of the embodiments of the same described or otherwise contemplated herein. In certain embodiments, the amphiphile comprises an amine functional head group. In certain embodiments, the amphiphile is TCD-NH₂ or PCD-NH₂

Methods of adsorbing a biomolecule in a sample (e.g., a biological sample from a subject) using the scaffolds hereof are provided. A method of adsorbing a biomolecule in a sample can comprise, for example, contacting, under adsorptive conditions, a sample with a surface of any of the scaffolds hereof, whereupon the biomolecule in the sample is adsorbed onto the molecular layer of the scaffold. It will be appreciated that such applications can be used to detect DNA in a sample, for example. The support material of the scaffold can be a polyacrylamide hydrogel. In certain embodiments, the amphiphile comprises an amine functional head group. The amphiphile can be TCD-NH₂ or PCD-NH₂. The biomolecule can be DNA.

Methods of adhering cells to a polymeric material are also provided. In certain embodiments, a method of adhering cells to a polymeric material comprises contacting cells with a polymeric material comprising a scaffold hereof (or, for example, a patterned layer of polymerized amphiphiles and one or more alkyl chains comprising a polymerizable DA group on a surface thereof, each of the polymerized amphiphiles comprising one or more functional head groups covalently bonded to the one or more alkyl chains and wherein: the one or more functional head groups are organized in a pattern comprising a width of about 1 nm at a periodicity of about 5 nm to about 10 nm, and positioned in an orientation capable of binding a target, and the DA groups are covalently bonded to an azide-functionalized or thiol-functionalized molecule in the absence of copper). In certain embodiments, the exposed polymer backbone of the molecular layer can comprise polydiacetylene covalently bonded to a thiol-functionalized and/or an azide-functionalized molecule in the absence of copper (i.e., the molecular layer can be secondarily functionalized).

The azide-functionalized or thiol-functionalized molecule can be a cell adhesion molecule. In certain embodiments, the cell adhesion molecule is a cRGD. The cells can be a mixture of cells. The cells can form a tissue.

In the above description, numerous specific details are set forth to provide a thorough understanding of the present disclosure. Particular examples may be implemented without some or all of these specific details and it is to be understood that this disclosure is not limited to particular biological systems or particular organs or tissues, which can, of course, vary but remain applicable in view of the data provided herein.

Additionally, various techniques and mechanisms of the present disclosure sometimes describe a connection or link between two components. Words such as attached, linked, coupled, connected, and similar terms with their inflectional morphemes are used interchangeably, unless the difference is noted or made otherwise clear from the context. These words and expressions do not necessarily signify direct connections but include connections through mediate components. It should be noted that a connection between two components does not necessarily mean a direct, unimpeded connection, as a variety of other components may reside between the two components of note. Consequently, a connection does not necessarily mean a direct, unimpeded connection unless otherwise noted.

Further, will be understood that the disclosure is presented in this manner merely for explanatory purposes and the principles and embodiments described herein may be applied to monolayers and materials that have configurations other than as specifically described herein. Indeed, it is expressly contemplated that the components of the monolayers and materials hereof be tailored in furtherance of the desired application thereof (e.g., for use with implants, regenerative medicine, chromatography studies, etc.).

All publications, patents, and patent documents referenced in this document are incorporated by reference. In the event of inconsistent usages between this document and those documents so incorporated by reference, the usage in the incorporated references should be considered supplementary to that of this document; for irreconcilable inconsistencies, the usage in this document controls.

### Certain Definitions

Unless defined otherwise, all technical and scientific terms used herein have the same meaning as commonly understood by one of skill in the chemical and biological arts. Although any methods and materials similar to or equivalent to those described herein can be used in the practice or testing of the subject of the present application, the preferred methods and materials are described herein. Additionally, as used in this specification and the appended claims, the singular forms “a”, “an” and “the” include plural referents unless the content clearly dictates otherwise. Thus, for example, where a compound/composition is substituted with “an” alkyl or aryl, the compound/composition is optionally substituted with at least one alkyl and/or at least one aryl.

When ranges are used herein for physical properties, such as molecular weight, or chemical properties, such as chemical formulae, all combinations and sub-combinations of ranges and specific embodiments therein are intended to be included.

Additionally, the term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +/−5% to 15% of the recited value) provided that one of ordinary skill in the art would consider equivalent to the recited value (e.g., having the same function or result). The term “comprising” (and related terms such as “comprise” or “comprises” or “having” or “including”) is not intended to exclude that in other certain embodiments, for example, an embodiment of any compound, composition of matter, composition, method, or process, or the like, described herein, may “consist of” or “consist essentially of” the described features. The term “substantially” can allow for a degree of variability in a value or range, for example, within 90%, within 95%, or within 99% of a stated value or of a stated limit of a range.

In describing representative embodiments, the disclosure may have presented a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

It is therefore intended that this description and the appended claims will encompass, all modifications and changes apparent to those of ordinary skill in the art based on this disclosure.

Any use of section headings and subheadings is solely for ease of reference and is not intended to limit any disclosure made in one section to that section only; rather, any disclosure made under one section heading or subheading is intended to constitute a disclosure under each and every other section heading or subheading.

Various modifications and variations of the described compositions, methods, and uses of the technology will be apparent to those skilled in the art without departing from the scope and spirit of the technology as described. Although the technology has been described in connection with specific exemplary embodiments, the invention as claimed should not be unduly limited to such specific embodiments. Indeed, various modifications of the described modes for carrying out the invention that are obvious to those skilled in the art are intended to be within the scope of the following claims.

The terms and expressions which have been employed are used as terms of description and not of limitation. In this regard, where certain terms are defined and are described or discussed elsewhere, the definitions and all descriptions and discussions are intended to be attributed to such terms. There also is no intention in the use of such terms and expressions of excluding any equivalents of the features shown and described or portions thereof.

Further, all publications and patents mentioned herein are incorporated by reference in their entireties for all purposes. In the event of inconsistent usages between this document and those documents so incorporated by reference, the usage in the incorporated reference should be considered supplementary to that of this document; for irreconcilable inconsistencies, the usage in this document controls.

### EXAMPLES

The following examples serve to illustrate the present disclosure. The examples are not intended to limit the scope of the claimed invention in any way.

### Example 1

### Materials and General Methods

Materials. 10,12-tricosadiynoic acid (10,12-TCDA, 97% purity) was purchased from GFS Chemicals (Powell, OH), dissolved in chloroform, and filtered using a 13-mm syringe filter with a Polytetrafluoroethylene (PTFE) membrane and 0.2-μm pores (VWR, Radnor, PA). 1,2-Bis(10,12-tricosadiynoyl)-sn-glycero-3-phosphoethanolamine (dPE, >99.0% purity) was purchased from Avanti Polar Lipids (Alabaster, AL) and used as received. Acrylamide (Aam, ≥99.9% purity), bisacrylamide (Bis), ammonium persulfate (APS), TEMED were purchased from Bio-Rad Laboratories (Hercules, CA).

Chloroform (≥99.5% purity), ethidium bromide (EtBr, 10 mg/mL in H₂O), 3-(trimethoxysilyl)propyl methacrylate (TMS-PMA), deoxyribonucleic acid sodium salt from salmon testes (dsDNA, 2000 bp), and phosphate-buffered saline (PBS, pH 7.4) were purchased from Sigma-Aldrich (St. Louis, MO) and used as received. Oxalyl chloride (>99% purity), lithium aluminum hydride (LiAlH₄, 95% purity), anhydrous dichloromethane (DCM, ≥99.8% purity), anhydrous diethyl ether (≥99.7% purity), and ammonium hydroxide solution (28-30%, NH₃ basis) were also all purchased from Millipore Sigma (St. Louis, MO) and used as received.

Isopropanol (IPA, ≥99.5% purity) and benzyl azide (Bn-N₃, ≥94% purity) were purchased from Fisher Scientific (Hampton, NH), and used as received. Ethanol (200 Proof) was purchased from Decon Laboratories, Inc. (King of Prussia, PA). Anhydrous sodium sulfate (Na₂SO₄, ≥99.0% purity), N,N-dimethylformamide (DMF, ≥99.8% purity), sodium dodecyl sulfate (SDS), and sodium hydroxide were all purchased from Fisher Scientific (Hampton, NH) and used as received. 10,12-tricosadiynoic acid (TCD-COOH, 97% purity) was purchased from GFS Chemicals (Powell, OH), dissolved in chloroform, and filtered using a 13-mm syringe filter with a PTFE membrane and 0.2 μm pores (VWR, Radnor, PA).

Acrylamide (Aam, 99.9% purity), N,N′-methylenebis(acrylamide) (Bis, 99% purity), TEMED, and ammonium persulfate (APS) were all purchased from Bio-Rad Laboratories (Hercules, CA) and used as received.

Unless otherwise specified, amphiphiles were deposited on 1 cm×1 cm highly oriented pyrolytic graphite (HOPG, MicroMasch, Watsonville, CA) substrates. Substrates were cleaved immediately prior to sample deposition. All initial steps in the transfer process were carried out under UV-filtered light to prevent unwanted polymerixation. Electroformed 40-μm square Nickel mesh was purchased from Precision Eforming (Cortland, NY). PELCO conductive liquid silver paint, standard SEM pin stub mounts, AFM specimen discs (alloy 430), and double-coated carbon conductive tape were purchased from Ted Pella (Redding, CA).

Cyclo[Arg-Gly-Asp-D-PheLys(Azide)](cRGD) was purchased from Vivtide LLC (Gardner, MA). Milli-Q water (≥18.2 MΩ·cm resistivity) was used whenever water was required in an experiment. Ultrahigh purity nitrogen (UHP N₂, 99.999% purity) was purchased from Indiana Oxygen Company (Indianapolis, IN). Embedding medium for cryotomy was purchased from Sakura Finetek (Torrence, CA).

### Methods Generally.

Langmuir-Schaefer transfer. Unless otherwise stated, monolayers of TCD-NH₂, PCD-NH₂, TCD-OH, and TCD-COOH were prepared via a previously reported Langmuir-Schaefer (L-S) conversion process using a custom-built, temperature controlled transfer stage, as described in Davis et al., Hierarchical noncovalent functionalization of 2D materials by controlled Langmuir-Schaefer conversion, Langmuir 34: 1353-1362 (2018), Hayes et al., Multimicrometer noncovalent monolayer domains on layered materials through thermally controlled Langmuir-Schaefer conversion for noncovalent 2D functionalization, ACS Applied Materials & Interfaces 9: 36409-36416 (2017), and Bang et al., Spatially controlled noncovalent functionalization of 2D materials based on molecular architecture, Langmuir 34: 5454-5463 (2018).

The stage was utilized in conjunction with a MicroTrough XL Langmuir-Blodgett trough (Kibron Inc., Helsinki, Finland). HOPG substrates were mounted on standard 12-mm diameter stainless-steel AFM specimen discs and positioned on a magnet recessed in the temperature-controlled stage.

Conductive carbon tape was used to affix the HOPG to the specimen disk surface to ensure temperature uniformity across the substrate surface. The temperature of the substrate was measured using a thermocouple prior to dipping.

Films of each amphiphile were assembled at the air-water interface by depositing the appropriate amount of a 0.075 mg/mL amphiphile solution in chloroform, on a subphase comprised of 40 mM CaCl₂) in Milli-Q water, maintained at 30° C.

For example, for TCD-NH₂ studies, TCD-NH₂ films were created at the air-water interface by depositing 36 μL of 0.75 mg/mL chloroform solution of the amphiphile on a subphase comprised of 40 mM CaCl₂ in Milli-Q water, maintained at 30° C. Following deposition, 15 minutes was allowed for the chloroform to evaporate. Barriers were then swept inward at 6.23 mm/min to achieve the target mean molecular area (MMA) (typically 30 Å²/chain); the feedback mechanism was subsequently switched to maintain the surface pressure measured at the point at which the target MMA was achieved. HOPG substrates were freshly cleaved, mounted on the thermally controlled stage, heated to 70° C. for full coverage monolayers, then lowered (at 2 mm/min) into contact with the subphase. Contact was maintained for 2 minutes, and the HOPG was then lifted out of contact at a rate of 2 mm/min.

dPE films were created at the air-water interface by depositing 40 μL of 0.6 mg/mL chloroform solution of dPE on a subphase of Milli-Q water maintained at 30° C. Following deposition, 30 minutes was allowed for the chloroform to evaporate. Trough barriers were then moved inward at 6.23 mm/min until a target surface pressure of 30 mN/m was achieved. HOPG substrates were freshly cleaved, mounted on a the thermally controlled stage, and heated to 70° C., then lowered (at 6 mm/min) into contact with the subphase. Contact was maintained for 2 minutes. The HOPG was then lifted out of contact at 2 mm/min.

After contact with the subphase was broken, HOPG substrates were blown dry with UHP N₂. Finally, unless otherwise stated, HOPG substrates were placed under a hand-held UV lamp (254 nm, 8 W) for 1 hour with about 10 cm between the lamp and the substrate to induce diyne photopolymerization.

Preparation and assembly of striped phase monolayer of TCDA and dPE on HOPG. Striped PDA (sPDA) monolayers of TCDA and dPE were prepared using a previously reported Langmuir-Schaefer (LS) conversion method described above. LS conversion was performed using a MicroTrough XL Langmuir-Blodgett trough (Kibron Inc., Helsinki, Finland). The film was assembled at the air-water interface by depositing chloroform solution of TCDA (0.75 mg/mL, 34 μL) or dPE (0.6 mg/mL, 41 μL) on a subphase of water (˜18 MΩ·cm) at 30° C. The chloroform was allowed to evaporate for 15 minutes before the trough barriers were slowly moved inward (6 mm/min barrier motion) until the target MMA (35 Å²/molecule for TCDA) or target pressure (30 mN/m) was achieved. A freshly cleaved HOPG substrate was mounted on an automated dipper attachment, with the HOPG oriented nearly parallel to the air-water interface (cleaved side facing down), then lowered horizontally at a constant rate of 6 mm/min. The temperature of substrates was held at 45° C. for TCDA and 70° C. for dPE monolayer. After 2 minutes of contact with the interface, the HOPG was gently lifted out of contact with the liquid at the same speed. After contact with the interface was broken, the substrate was blown dry with UHP N₂.

AFM imaging. All AFM measurements were performed using a Bruker (Bruker Instruments, Billerica, MA) MultiMode AFM, equipped with an E scanner, under ambient conditions, utilizing Bruker RFESP-75 tips (nominal force constant 3 N/m and radius of curvature <10 nm). Micrographs were collected in tapping mode with a resolution of either 512 or 1024 data points/line.

Image analysis. Images were processed using the Java-based image processing software ImageJ (NIH, Bethesda, MD) and Gwyddion SPM scanning probe microscopy data visualization and analysis software. Plane flattening, median line corrections, scar artifact removal, contrast adjustment and pixel counting were corrected using appropriate software modules. Relative coverage was estimated by digital image analysis with ImageJ after scale normalization for quantification of 40× fluorescence micrographs. A representative area of 20 μm² was identified for region of interest (ROI) analysis of pixel intensity values. For each fluorescence data point graphed, 3 samples were prepared and 6 images were analyzed per sample. Pixel intensity values were averaged, and corresponding standard deviations were determined and reported for the data point tabulated.

SEM imaging. SEM imaging under high magnification was performed using a Teneo VS SEM (FEI Company, Hillsboro, Oregon) at a working distance of about 7 mm, using the segmented in-lens T3 secondary electron (SE) detector. An image acquisition beam current of 0.4 nA was typically utilized with a 32-μm diameter aperature at an accelerating voltage of 5 kV. Substrates were mounted with conductive carbon tape to standard SEM pin stub specimen mounts. A small amount of colloidal silver paint (PELCO®, Ted Pella, Inc.) was applied along the perimeter of the HOPG to enhance substrate-mount conductivity. All images were collected with a 1536×1024 pixel resolution and a 30-μs dwell time, unless otherwise specified.

Molecular modeling. Software packages Maestro and Macromodel (Schrödinger, Cambridge, MA) were used, respectively, to visualize molecular structures and to perform force field minimizations. Energy minimizations on all models were performed using the OPLS_2005 force field, with extended cutoffs for van der Waals, electrostatic, and hydrogen-bonding interactions. Minimizations were performed using the Polak-Ribiere conjugate gradient (PRCG) algorithm and gradient method with 50,000 runs and a convergence threshold of 0.05.

Preparation of PDMS stamps. Polydimethylsiloxane (PDMS) stamps for microcontact printing were prepared using a modification of the protocol described in Thakar & Baker, Lithography-free production of stamps for microcontact printing of arrays, Analytical Methods 2: 1180-1183 (2010). Briefly, a SYLGARD 184 silicone elastomer base and curing (crosslinking) agents were combined at a 10:1 m/m ratio and thoroughly mixed. A 40-μm square Nickel mesh was cut into 1 cm² pieces and affixed, using double-sided tape, onto a glass Petri dish. The PDMS mixture was poured over the Nickel mesh, then deaerated in a vacuum desiccator until no bubbles remained. The PDMS was cured overnight at 60° C.; the underlying Nickel mesh pieces were then gently peeled from the PDMS, generating a micropatterned surface. Micropatterned stamps were cleaned by sonication in a 1:1:1 (v/v/v) mixture of ethanol, methanol, and Milli-Q water for 1 hour, and subsequently placed in an oven for an additional 1 hour at 60° C. to allow residual polar solvent mixture to evaporate. The micropatterned stamps were then soaked in hexanes for 6 hours, replacing the solvent with fresh hexanes every 2 hours. Finally, the micropatterned stamps were dried overnight at ° C. and stored covered in a petri dish, pattern side up, prior to use.

Microcontact printing (μCP) of dPE. Inking and printing steps were performed following the procedure described in Davis et al., Hierarchically patterned striped phases of phospholipids: toward controlled presentation of carbohydrates, Faraday Discussions 219: 229-243 (2019). Briefly, the molecular ink solution was prepared at 2.5 mg/mL in CHCl₃ and diluted to 0.4 mg/mL with ethanol. Prior to inking, the PDMS stamp was rinsed with ethanol and blown dry with UHP N₂. The stamp was then immersed for 1 minute in the ink solution, removed and blown dry with UHP N₂, and placed pattern side up at room temperature for 1 hour. The pattern side of the PDMS stamp was brought into contact with a freshly cleaved HOPG substrate for 1 minute and carefully lifted off the surface. The microcontact printed HOPG substrate was then exposed to UV (to induce diyne photopolymerization) in a UVP CL-1000 Ultraviolet crosslinker device (254 nm, 8 W) for 1 hour with about 10 cm between the lamp and substrate surface.

Covalent Transfer of PDA Striped Phases from HOPG to PAAm.

Preparation of vinyl functionalized coverslips. Surfaces were grafted with 3-(trimethoxysilyl)propyl methacrylate (TMS-PMA) using the procedures described in Yuk et al., Tough bonding of polymeric materials to diverse non-porous surfaces, Natural Materials 15: 190-196 (2016).

This approach generated surfaces displaying vinyl groups capable of participating in the polymeric material polymerization, creating covalent linkages between the gel and the glass surface. Glass coverslips were first cleaned of residual organic material by sonication in a 1:1:1 (v/v/v) mixture of ethanol, isopropanol, and water for 30 minutes. Coverslips were then rinsed vigorously with ethanol and dried using UHP N₂. To activate the glass surface with hydroxyl (OH) groups, the slides were treated by oxygen plasma (30 W at a pressure of 200 mtorr; Harrick Plasma, model PDC-XG3) for 5 minutes on each side. Immediately following plasma cleaning, the glass coverslips were submerged in 40 mL or 100 mL of silane solution (100 mL water, 10 μL acetic acid (used to adjust the solution pH to 3.5), and 2 wt % TMS-PMA) and incubated for 2 hours or overnight (ca. 12 hours), respectively, at room temperature. Finally, the substrates were thoroughly rinsed with ethanol and water, in triplicates, then dried and stored in a low-humidity environment prior to use.

Preparation of polymeric material precursor solution. The polyacrylamide (PAAm) monomer mixture was prepared by following the protocol reported in Denisin & Pruitt, Tuning the range of polyacrylamide gel stiffness for mechanobiology applications, ACS Applied Materials & Interfaces 8(34): 21893-21902 (2016). PAAm polymeric materials (hydrogels) were prepared according to formulations reported in Tse & Engler, Preparation of polymeric material substrates with tunable mechanical properties, Current Protocols in Cell Biology 47: 10.16.11-10.16.11 (2010). Briefly, to prepare monomer/crosslinker precursor solutions, acrylamide (AAm) and bis-acrylamide (Bis) were dissolved together in 5 mL of Milli-Q water, using compositions described herein based on the total concentration of AAm/Bis (w/v) in the gel mixture (% T), and based on the crosslinker-to-monomer ratio (% C):

$$\begin{matrix}\% T = \frac{AAm ( g ) + Bis ( g )}{\text{Total vol} ( mL )} \times 100 \\ \% C = \frac{Bis ( g )}{AAm ( g ) + Bis ( g )} \times 100\end{matrix}$$

Solutions were ultrasonicated for at least 15 minutes (or until all the solid was dissolved) and then sealed with parafilm and stored at room temperature.

For example, to prepare PAAm with a formulation of 30% T and 5% C, acrylamide (Aam) (3.910 g) and bisacrylamide (Bis) (0.205 g) were added to water (13.7 mL). Precursor solution was ultrasonicated until all the solid was dissolved.

Preparation of HOPG substrate for polymeric material gelation. For PAAm curing and sPDA monolayer exfoliation, the monolayer (e.g., sPDA)/HOPG samples were first covered with rubber molds 2 cm in diameter and 2 mm thick (Ted Pella, Inc.) with an 0.5 cm×0.5 cm square cut to fit the HOPG area. Double-sided clear tape (3M Scotch) was used to create a temporary seal between the HOPG and the rubber mold, preventing polymeric material precursor solution from leaking.

Exposure of HOPG substrate to precursor solution. Precursor solution was degassed by bubbling with UHP N₂ for 5 minutes prior to gelation. A 10% w/v solution of the ammonium persulfate (APS) initiator was prepared in Milli-Q water. The catalyst, TEMED, was used as received. To initiate gelation, 10 μL of the APS solution, 1 μL of TEMED, and 989 μL of the chosen AAm/Bis precursor solution were mixed by gentle pipetting, and vortexed thoroughly for 30 seconds. Subsequently, 100 μL of the precursor mixture were cast on the prepared HOPG substrates, and the mixture was immediately capped with a 1 cm² vinyl-functionalized glass coverslip, creating a sandwich-like arrangement and ensuring that the entire HOPG surface was covered by the acrylamide mixture. Polymeric materials were cured at room temperature for 15 minutes, and stored in Milli-Q water, unless otherwise specified. Hydrogels were cured at room temperature for at least 1 hour prior to further use.

Reaction of cRGD with PDA. A stock solution (0.5 mg/mL) of cRGD was prepared in PBS buffer (50 mM, pH 7.4; 1 mM CaCl₂)). This stock solution was diluted to 20 μg/mL in PBS buffer. Next, 600 μL of cRGD solution (20 μg/mL) were added to each PAAm substrate to cover the surface completely. The substrates were incubated using a shaker for 4 hours at room temperature. Each PAAm substrate was washed three times with water and two times with PBS buffer for 30 seconds per cycle to remove any nonspecifically adsorbed cRGD.

Reaction of benzyl azide with PDA. A stock solution (1 mg/mL) of Bn-N₃ was prepared in ethanol. This stock solution was diluted to 20 μg/mL in water. Next, 800 μL of Bn-N₃ solution (20 μg/mL) was added to each PAAm substrates to cover the surface completely. The substrates were incubated using a shaker for 4 hours at room temperature. Each PAAm substrate was washed with ethanol three times with ethanol and two times with water 10 seconds per cycle to remove any nonspecifically adsorbed Bn-N₃.

C2C12 cell culture. C2C12 cells were passaged twice before substrate exposure. Substrates were seeded with 6,000 cells for 24 hours. Cells were then fixed with paraformaldehyde and blocked with 1% BSA. Phalloidin staining was carried out using 2 μL of 4× Alexa fluor 568 (A568) in 800 μL 0.1% BSA. Nuclei were stained with 800 μL DAPI in 0.1% BSA (1 μL DAPI/mL staining solution). Substrates were stored in phosphate-buffered saline (PBS) buffer prior to imaging by confocal microscope.

For cell adhesion studies, C2C12 cells were cultured in Advanced DMEM containing 10% FBS, 1× penicillin/streptomycin, and 1% glutagro supplement at 37° C. in 5% CO₂. Cells were seeded on sterilized PDMS at 2.5×10⁴ cells/mL and grown to 70% confluence before switching to a 2% horse serum differentiation media. To analyze cell adhesion, spreading and differentiation, cells were fixed and stained with DAPI (1:1000) and phalloidin (1:200).

DNA adsorption experiments. Initially, a 1 mg/mL stock solution (2500 μM) of ethidium bromide (Eb) in Milli-Q water was prepared and diluted to 100 μM with Milli-Q water (see, e.g., the protocol described in Chib et al., Associated anisotropy decays of ethidium bromide interacting with DNA, Methods Applied Fluorescence 2(1): 015003 (2014).

Intercalation of ethidium bromide (Eb) in double stranded DNA (dsDNA). The ratio of dsDNA sodium salt from salmon testes (2000 base pairs long, ˜680 nm) to Eb was adjusted to produce a ratio of ca. 20 Eb molecules/DNA strand. For instance, to prepare a 1 μM DNA and 20 μM Eb solution, 2.60 mg of dsDNA were measured into a vial, 40 μL of Eb solution was added, and additional Milli-Q water was added to achieve a final volume of 2 mL. The mixture was sonicated for 1 hour, mixed thoroughly by vortex for at least 5 minutes, and allowed to further equilibrate overnight. This solution was stored at −20° C. when not in use.

Sample preparation. For drop-casting tests, 100 μL of the corresponding Eb-DNA solution was cast on the TCD-NH₂@PAAm hydrogels and incubated for 10 minutes in a petri dish protected from light and dust. Following incubation, the Eb-DNA solution was carefully wicked off the hydrogel surface using a kimwipe, followed by three rinsing cycles of 30 seconds each, with PBS buffer pH 7.4. Samples were stored in PBS buffer (pH 7.4) and analyzed within 1 hour after the adsorption experiment to minimize desorption.

Eb-intercalation in dsDNA. The components were weighted together and diluted to the corresponding concentration with Milli-Q water, and further sonicated for 1 hour. This solution was left at room temperature to equilibrate overnight prior to use.

Eb-DNA adsorption on TCD-NH₂ monolayers. The functionalized hydrogels were equilibrated by soaking them in Milli-Q water (PBS buffer pH 7.4) overnight. Briefly, 100 μL of the Eb-DNA solution was deposited on each substrate and thereafter incubated at room temperature for 10 minutes in a container to protect the hydrogels from light and dust. Subsequently, the substrates were rinsed 3 times with PBS buffer (pH 7.4) The swollen equilibrated hydrogel substrates were imaged within the next hour, using confocal microscopy imaging (λₑₓ 480 nm for an λₑₘ of 620 nm), which significantly reduced drifting in the focal plane. Note that Eb-NDA@TCD-NH₂—PAAm in solution represents analyte desorption over time.

Wide-field fluorescence microscopy. Wide-field fluorescence micrographs were obtained using an Olympus BX-51 optical microscope with an Olympus DP71 color camera. Images were acquired using either a 10× (metallographic, plan achromatic, NA=0.25, infinity-corrected optics), 40× (metallographic, plan-fluorite aberration correction, NA=0.75, infinity-corrected optics) or a 100× (metallographic, apochromatic, and flat field correction, NA=0.95) brightfield objective. A UMWB2 filter cube was utilized (460-490 nm excitation band-pass filter, dichroic filter wavelength of 500 nm, long-pass emission filter wavelength of 520 nm). The dwell times for imaging were kept at 1 second for 10× and 40× images, and 4 seconds for 100× images, with 3 accumulations taken under integration capturing mode. Image resolution was 1024×1024 pixels, unless otherwise specified.

Confocal fluorescence microscopy and spectral imaging. Confocal micrographs and associated spectra were acquired utilizing a Zeiss LSM 880, Axio Examiner upright confocal microscope. Samples were immersed in pH 7.4 PBS buffer for at least 24 hours prior to imaging to minimize sample motion due to dynamic swelling under imaging conditions. Unless otherwise stated, excitation was provided by a 488-nm Ar laser set to 100% power. Data were obtained using a 20× objective (plan-apochromat, DIC M27 75 mm, NA=1.0) and a 32-channel GaAsP spectral photomultiplier tube detector. Micrographs and corresponding spectra were obtained at a resolution of 512×512 pixels, a bit depth of 8 bits, a dwell time of 16.38 μs/pixel (using unidirectional scanning and averaging 16 times per line), and a pinhole set to 1 Airy unit (A.U.). For detection, 16 of the 32 channels were used to collect data, with bins centered at values from 531-664 and a resolution (bin width) of 8.9 nm.

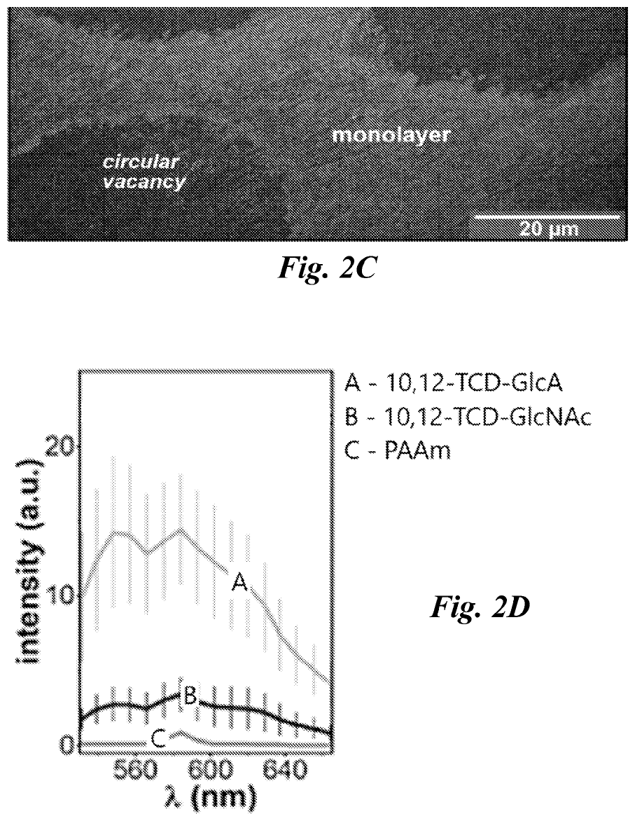

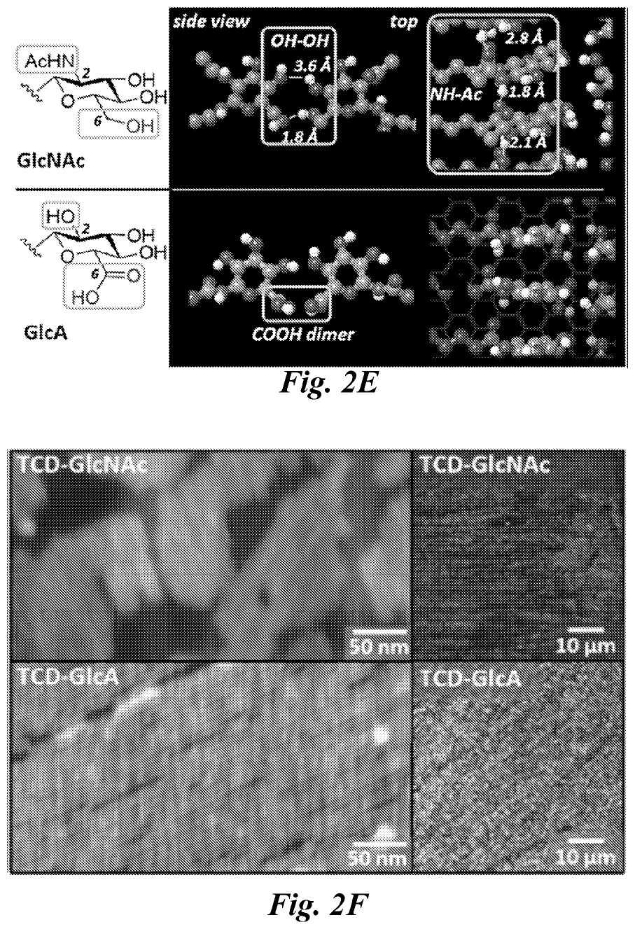

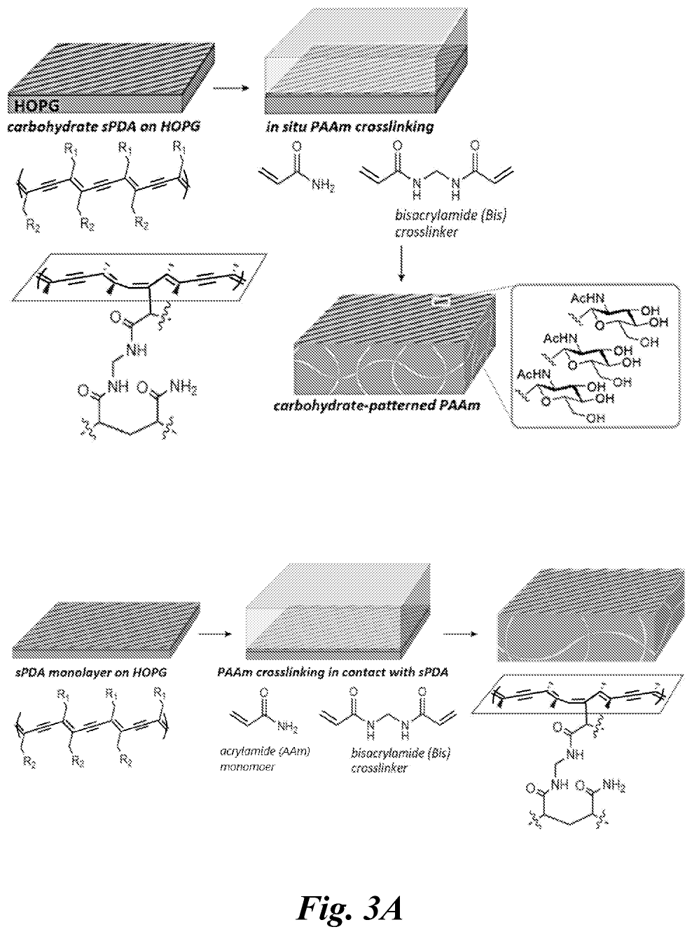

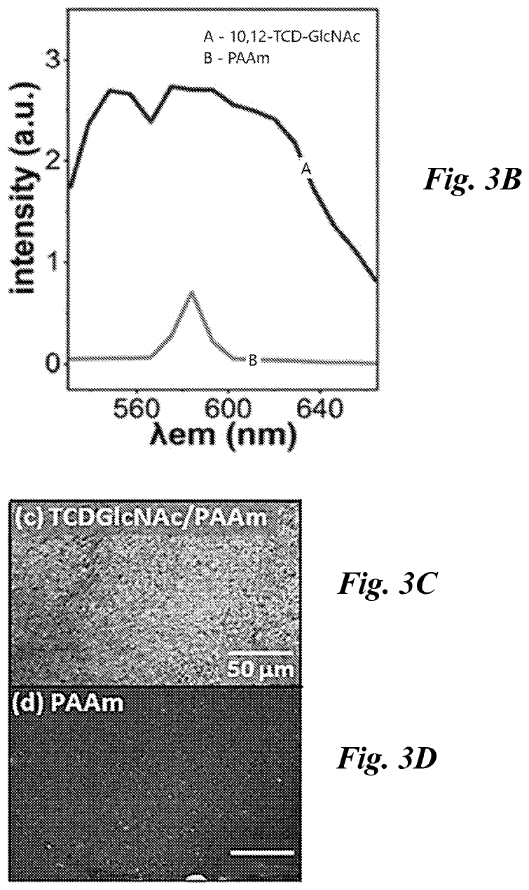

Larger AFM and SEM images illustrating LS assembly of striped phase films of dPE and TCD-NH₂ on HOPG. FIGS. 2B-2D, 3B-3F, and 4B-4D show AFM and SEM images illustrating the lamellar and domain structure for stiped phase polydiacetylene (PDA) films. FIGS. 3E and 3F show larger AFM and SEM images, respectively, illustrating nanoscale and microscale molecular ordering achieved using thermally regulated Langmuir-Schaefer conversion. In FIG. 3E, AFM images of TCD-NH₂ exhibit striped phase domains with edge lengths >100 nm. Also visible in the images are long linear features generally crossing the entire image from left to right, which correspond to HOPG step edges (an example is labeled in FIG. 3E).

In FIG. 3F, SEM images provide microscale information on assembled TCD-NH₂ striped phase domain morphology on HOPG. Long linear defects evolve in ordered monolayers during polymerization due to conformational changes when the diacetylene rehybridizes to form the polydiacetylene; these defects are emphasized in the SEM electron beam. Linear defect orientation can be used to infer molecular row orientation.

### Example 2

### Synthesis of Amphiphiles

Synthesis of 4,6-TCD-COOH. Synthesis was carried out using a modification of the procedures set forth in Mowery & Evans, The synthesis of conjugated diacetylene monomers for the fabrication of polymerized monolayer assemblies, Tetrahedron Letters 38(1): 11-14 (1997). Briefly, a solution of 1-octadecyne (1.0 equiv, 3.7 mmol), NBS (1.1 equiv, 4.1 mmol), and silver nitrate (0.1 equiv, 0.37 mmol) in acetone (50 mL) was stirred at room temperature for 3 hours. Acetone was then removed by a rotary evaporator. The resulting product was dissolved in heptane. Subsequently, water (30 mL) was added. The organic fraction was extracted and dried over anhydrous sodium sulfate (Na₂SO₄). The heptane was removed under reduced pressure to yield 1-bromo-1-octadecyne as a white solid. Next, 4-pentynoic acid (1 equiv, 4.2 mmol) was dissolved in 1 M KOH (1.8 equiv, 6.8 mmol) and methanol (10 mL). Next, CuI (0.25 equiv, 1.1 mmol) dissolved in ethylamine solution (10 mL) and hydroxylamine hydrochloride (0.1 equiv, 0.42 mmol) was added, which turns the reaction mixture yellow. The reaction mixture was then cooled to −78° C.

A solution of 1-bromo-1-octadecyne (1 equiv, 3.8 mmol) dissolved in THF (10 mL) was then added dropwise. The reaction was allowed to warm to room temperature and stirred overnight. If the solution turned blue, additional aliquots of hydroxylamine hydrochloride were added to the reaction mixture. The reaction was quenched with an aqueous solution of 30% sulfuric acid. The solution was extracted with diethyl ether (3×15 mL) and washed with water (3×15 mL) and brine (3×15 mL), respectively. The organic layer was dried over anhydrous Na₂SO₄. The solvent was removed under reduced pressure. The crude 4,6-tricosadiynoic acid (4,6-TCDCOOH) was recrystallized from heptane.

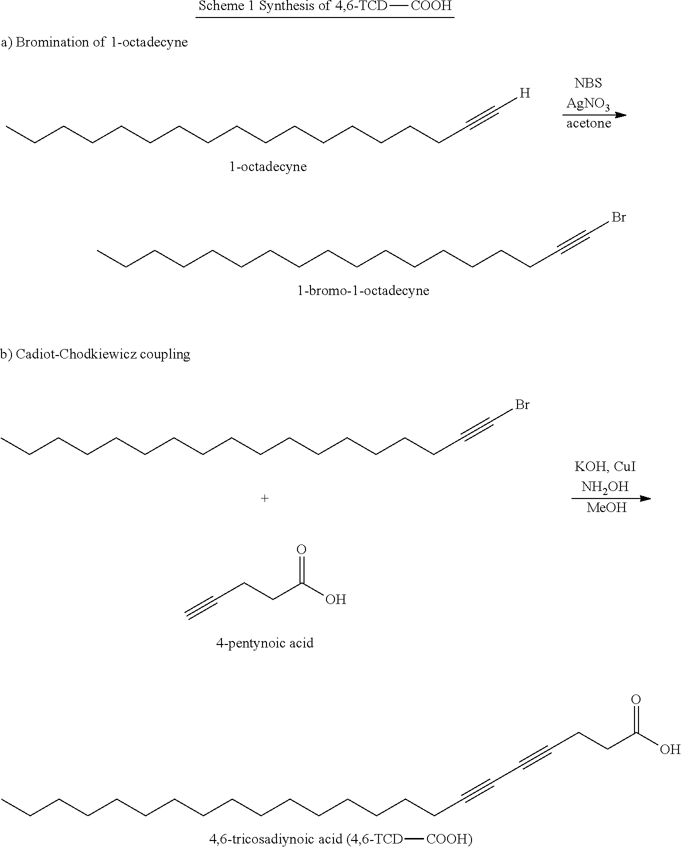

Synthesis of 4,6-TCD-COCl and 10,12-TCD-COCl. Synthesis was performed utilizing the method set forth in Lee et al., A reversibly mechanochromic conjugated polymer, Chemical Communications 55(63). 9395-9398 (2019). First, the diynoic acid (1 equiv, 2.9 mmol) was dissolved in anhydrous DCM under N₂ atmosphere. Oxalyl chloride (1.3 equiv, 3.7 mmol) and two drops of N,N-dimethylformamide (DMF) were added to the solution. The reaction mixture was stirred at room temperature overnight and concentrated under reduced pressure to obtain the diynoyl chloride as a yellow oil, which was used for the next step without further purification.

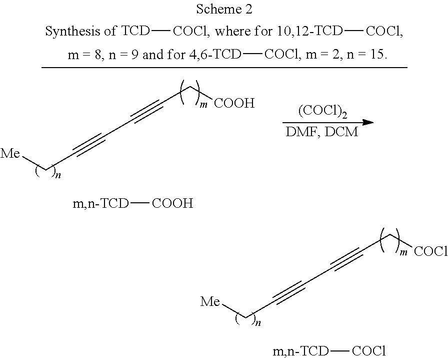

Synthesis of 10,12-TCD-GlcA. 10,12-TCD-GlcA was prepared by modification of the procedures set forth in Pitt et al., Synthesis of a glucuronic acid and glucose conjugate library and evaluation of effects on endothelial cell growth, Carbohydrate Research 339(11): 1873-1887 (2004) and Tosin & Murphy, Synthesis of alpha-glucuronic acid and amide derivatives in the presence of a participating 2-acyl protection group, Organic Letters 4921): 3675-3678 (2002). D-glucuronic acid (GlcA, 1 equiv, 7.7 mmol) was added to acetic anhydride (23 mL), and the resulting suspension was stirred at 0° C. for 10 minutes. Next, iodine (0.053 equiv, 0.41 mmol) was added and the resulting mixture was stirred at 0° C. for 2 hours, and subsequently at room temperature for 3 hours. After the reaction was complete, excess acetic anhydride was removed under reduced pressure. The resulting mixture was extracted with DCM (40 mL) and washed with 1 M Na₂S₂O₃ (3×40 mL). The organic layer was dried over anhydrous Na₂SO₄. The solvent was removed under reduced pressure to obtain OAc-GlcA as white solid.

OAc-GlcA (1 equiv, 3.3 mmol) was dissolved in anhydrous methanol (15 mL), and the resulting solution was refluxed for 24 hours. The excess methanol was removed under reduced pressure and the crude product (OMe-GlcA) was recrystallized from methanol. Next, OMe-GlcA (1 equiv, 2.5 mmol) was dissolved in anhydrous DCM under N₂ atmosphere, and TMS-N₃ (2.5 equiv, 6.2 mmol) and SnCl4 (0.5 equiv, 1.2 mmol) were added to it under N₂ atmosphere. The reaction mixture was stirred for 15 hours at room temperature and, thereafter, the reaction mixture was diluted with DCM (15 mL). Saturated sodium bicarbonate solution was added, and the mixture was vigorously stirred for 30 minutes. The organic layer was poured into water and twice extracted with DCM. The combined organic layer was washed with brine and solvent was evaporated under reduced pressure. Crude OMe-GlcA-N₃ was recrystallized in ethyl acetate and hexane (1:1). OMe-GlcA-N₃ (1 equiv, 1.3 mmol) and 10,12-TCD-COCl (1.2 equiv, 1.5 mmol) were dissolved in anhydrous DCM (15 mL) under N₂ atmosphere. Triphenylphosphine (1.3 equiv, 1.7 mmol) was dissolved in anhydrous DCM (5 mL), and the resulting solution was added to the reaction mixture dropwise. The reaction was stirred at room temperature for 15 hours. Subsequently, the mixture was diluted with CHCl₃ (20 mL), washed once with a saturated aqueous solution of NaHCO₃ (10 mL) and then with water (10 mL) until neutral pH was achieved. After drying over Na₂SO₄, the solution was filtered, then evaporated to dryness, to obtain 10,12-OMe-TCD-GlcA.

A solution of 0.05 M lithium hydroxide was prepared in 2.5:1:0.5 methanol-water-THE. 10,12-OMe-TCD-GlcA (1 equiv, 0.3 mmol) was dissolved in 0.05 M lithium hydroxide solution (6 equiv.), and the solution was stirred at 0° C. for 3 hours. The mixture was diluted with water and pH was adjusted to 3 with Amberlite® IR 120 H. The Amberlite was removed by filtration and the solvent was removed under reduced pressure. The resulting aqueous solution was freeze dried to yield 10,12-TCD-GlcA as white solid.

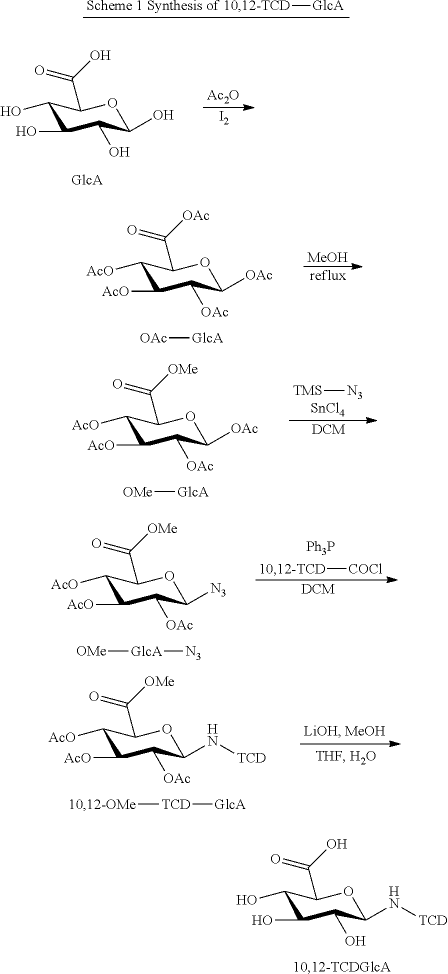

Synthesis of 10,12-TCD-OH. Synthesis was performed utilizing the procedures set forth in Clarisse et al., Aqueous 1,3-dipolar cycloadditions promoted by copper nanoparticles in polydiacetylene micelles, Green Chemistry 19(13): 3112-3115 (2017). Briefly, 10,12-TCD-COOH (1 equiv) was dissolved in anhydrous diethyl ether (Et₂O). The resulting solution was cooled to 4° C. Then, lithium aluminum hydride (LiAlH₄, 3 equiv) was added. The reaction mixture was stirred for 4 hours at room temperature. After completion of the reaction, the mixture was cooled to 4° C. in an ice bath. The reaction was quenched with an aqueous solution of sodium hydroxide (15% w/w), followed by water. The resulting orange precipitate was filtered off on celite, and the filtrate was washed with aqueous 10% w/v hydrochloric acid. The organic phase was dried over anhydrous Na₂SO₄ and concentrated under reduced pressure to obtain 10,12-TCD-OH as a white solid.

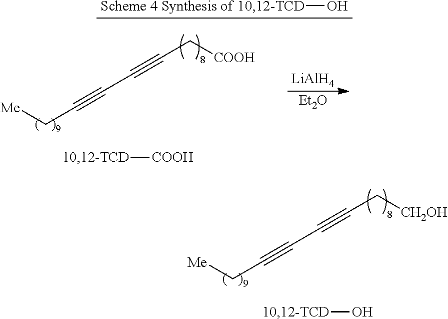

Synthesis of 10,12-diynamine from a 10,12-diynoic acid. 10,12-pentacosadynamine (PCD-NH₂) and 10,12-tricosadiynamine (TCD-NH₂) were both prepared from the structurally analogous 10,12-diynoic acid (i.e., 10,12-pentacosadiynoic acid (PCD-COOH) and 10,12-tricosadiynoic acid (TCD-COOH) using a modification of the procedures reported in Howarth et al., Lipophilic peptide nucleic acids containing a 1,3-diyne function: synthesis, characterization and production of derived polydiacetylene liposomes, Tetrahedron 61: 8875-8887 (2005) and Lee et al., A reversibly mechanochromic conjugated polymer, Chemistry Communications 55: 9395-9398 (2019).

Briefly, 10,12-diynoic acid (1 eq) (e.g., TCD-COOH or PCD-COOH, respectively) was dissolved in anhydrous DCM under N₂ atmosphere. Oxalyl chloride (1.3 eq) and N,N-dimethylformamide (DMF) (2 drops) were added to the solution. The reaction mixture was stirred at room temperature overnight and concentrated under reduced pressure to obtain 10,12-diynoyl chloride as a yellow oil, which was used for the next step without further purification.

Next, in a round-bottom flask, 28-30% aqueous ammonium hydroxide (1.3 eq) was added. 10,12-diynoyl chloride (1 eq) was dissolved in THF and the resulting solution was added to the ammonium hydroxide solution at 0° C. The reaction mixture was stirred at room temperature for 6 hours. The product was extracted with DCM (3×50 mL) and the combined organic extract was dried over anhydrous Na₂SO₄. The DCM was evaporated under reduced pressure to yield 10,12-tricosadiynoyl amide as a white solid. 10,12-diynoyl amide (1 eq) was placed in a round-bottom flask. Anhydrous diethyl ether was added to the flask under N₂ atmosphere, yielding a white suspension. Subsequently, LiAlH₄ (10 eq) was added to the suspension at 0° C. and the reaction mixture was stirred at room temperature for 20 hours. After the reaction was complete, the mixture was cooled to 0° C. and treated with the sequential dropwise addition of water, aqueous NaOH (15% w/w) and water. The mixture was filtered to remove inorganic impurities, and the filtrate was dried over anhydrous Na₂SO₄. After drying over Na₂SO₄, the solvent was evaporated under reduced pressure to yield the final product (TCD-NH₂). TCD-NH₂ was dissolved in chloroform and filtered using 13-mm syringe filters with PTFE membranes and 0.2-μm pores (VWR, Radnor, PA) prior to use.

Synthesis of 10,12-TCD-GlcNAc and 4,6-TCD-GlcNAc. To examine the role of the alkyl chain segment proximal to the head group in spatially constraining carbohydrates for multivalent binding, two isomeric N-acetylglucosamine (GlcNAc) amphiphiles (10,12-tricosadiynoic acid (10,12-TCD-GlcNAc) and 4,6-tricosadiynoic acid (4,6-TCD-GlcNAc)) were synthesized. 4,6-TCD-GlcNAc positions the diyne much closer to the carbohydrate head group, which could result in stronger ligand clustering and, thus, potentially increase K_{D}.

Amphiphiles with GlcNAc and GlcA head groups were prepared through modifications of procedures reported in Gyorgydeak et al., Synthesis of N-(beta-D-glucopyranosyl) and N-(2-acetamido-2-deoxy-beta-D-glucopyranosyl) amides as inhibitors of glycogen phosphorylase, Bioorganic & Medicinal Chemistry 12: 4861-4870 (2004). First, 4,6-TCD-GlcNAc was synthesized by Cadiot-Chodkiewicz coupling. Briefly, to generate amphiphiles with GlcNAc-modified head groups, 4,6-trimethylsilyl chlorodifluoroacetate (TCDA) and (commercially-available) 10,12-TCDA were treated with oxalyl chloride (COCl)₂ in the presence of a catalytic amount of DMF to obtain 4,6-TCDCl and 10,12-TCDCl, respectively (see FIG. 1G). This process activates the carbonyl group towards nucleophilic substitution. Conjugation of GlcNAc to the diynoyl chloride (e.g., TCD-Cl) was performed using trimethyl phosphine (PMe₃)-mediated Staudinger reaction (FIG. 1A). Finally, the O-acetyl protecting groups were removed from the conjugated intermediate by Zemplen deacetylation.

More specifically, 2-Acetamido-3,4,6-tri-O-acetyl-2-deoxy-β-D-glucopyranosyl azide (GlcNAc-N3, 1 equiv, 0.14 mmol) was dissolved in anhydrous DCM (20 mL) and trimethyl phosphine (PMe₃, 1.2 equiv, 1.7 mL, 1 M THF solution) was added dropwise under N₂ atmosphere. After 10 minutes, TCD-COCl (1.1 equiv, 0.16 mmol) was added, and the mixture was stirred at room temperature overnight. The volatiles were removed under reduced pressure, and the crude product crystallized from ethanol. The acetyl-protected compound (1 equiv, 0.18 mmol) was dissolved in anhydrous methanol and sodium methoxide (0.1 equiv, 0.02 mmol, 0.5 M in methanol) was added. The reaction mixture was stirred at room temperature for 3 hours. Amberlite® IRC 120 H was added to neutralize the reaction mixture. The product was isolated as a precipitate from the solution.

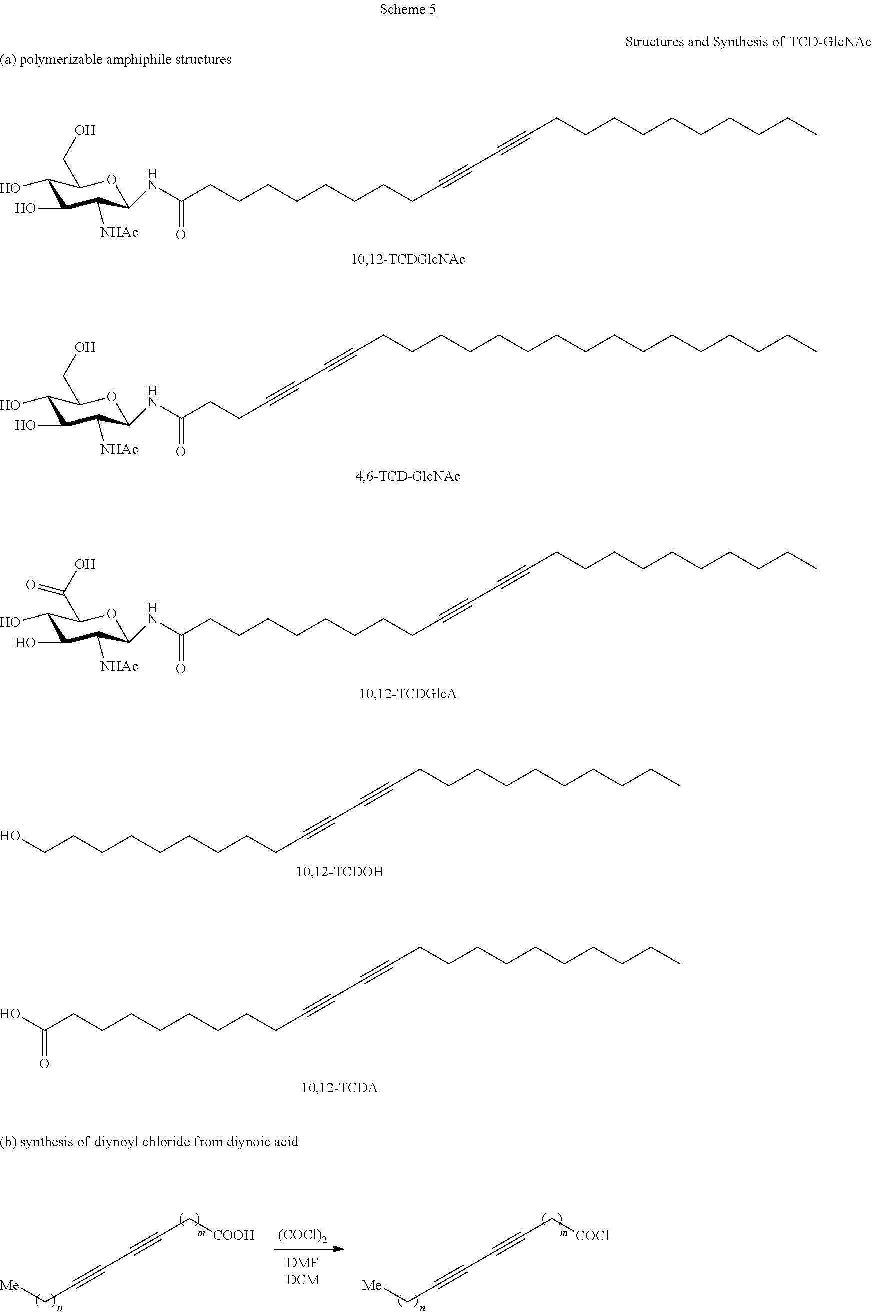

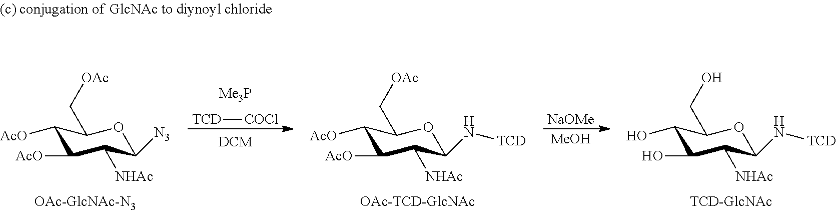

### Example 3

### Preparation and Assembly of sPDA Monolayers on HOPG

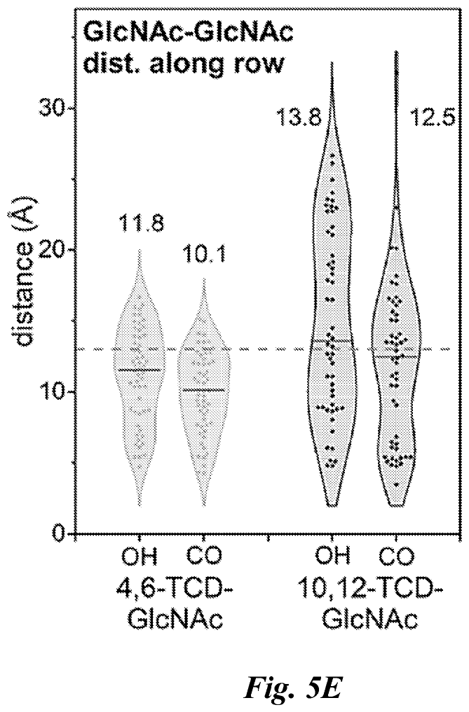

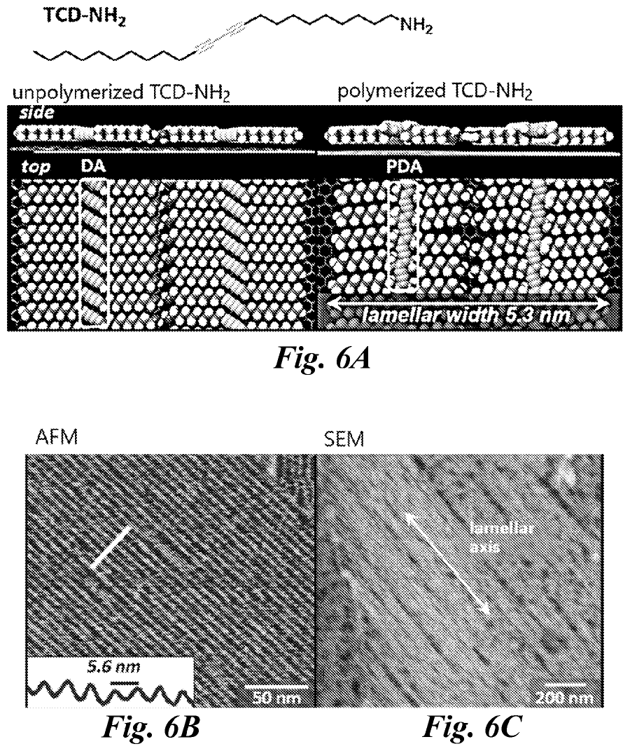

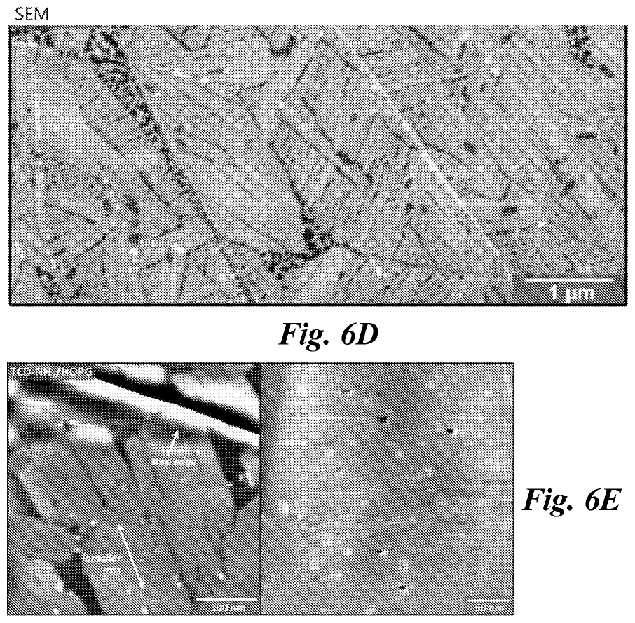

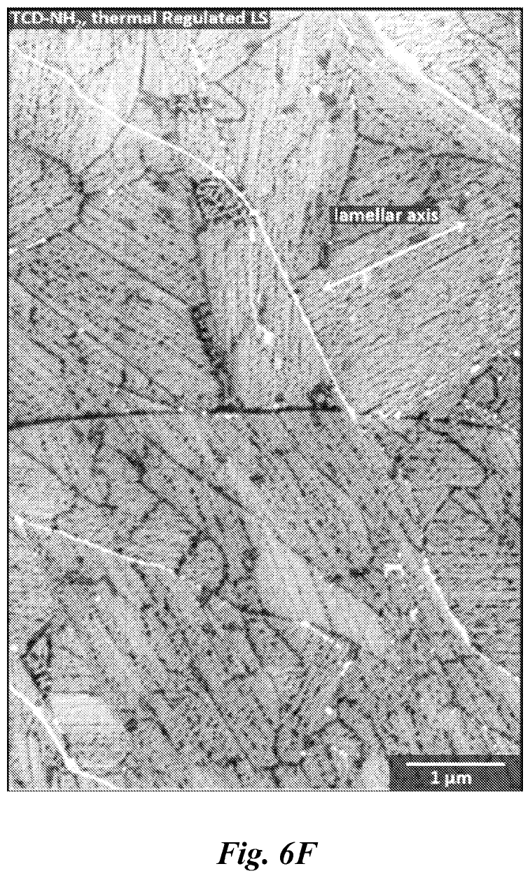

Striped polydiacetylene (sPDA) monolayers of polymerizable amphiphiles on HOPG (FIGS. 1A-1B and 6A-6D) were prepared via L-S conversion using a custom-built temperature-controlled transfer stage. Davis et al. (2018), supra; Bang et al. (2018), supra; and Hayes et al. (2017), supra. The stage was utilized in conjunction with a MicroTrough XL Langmuir-Blodgett trough (Kibron Inc., Helsink, Finland).

Briefly, L-S conversion begins with assembly of a standing phase Langmuir film of amphiphiles on an aqueous subphase. Freshly cleaved HOPG substrates were mounted on standard 12-mm diameter stainless steel atomic force microscopy (AFM) specimen discs and positioned on a magnet recessed in the temperature-controlled stage. Conductive carbon tape was used to affix the HOPG to the specimen disk surface to ensure temperature uniformity across the substrate surface. The temperature of the substrate was measured using a thermocouple prior to dipping.

Films of each amphiphile were assembled at the air-water interface by depositing the appropriate amount of a 0.75 mg/mL amphiphile solution in chloroform, on a subphase comprised of 40 mM CaCl₂ in Milli-Q water, maintained at 30° C. Barriers were then swept inward at 6.23 mm/min to achieve the target MMA; the trough feedback mechanism was set to maintain the target MMA. HOPG substrates were freshly cleaved, mounted on the thermally controlled stage, set at either 25° C. for transfer of monolayers containing circular vacancies, or 70° C. for full coverage monolayers, then lowered (at 2 mm/min) into contact with the standing phase monolayer (i.e., L-S film) and a subset of molecules on the L-S film, causing some molecules within the film to transfer to the HOPG surface and convert from the standing arrangement on the subphase, to a lying-down orientation on the HOPG, with their alkyl chains parallel to the substrate. Contact was maintained for 2 minutes, and the HOPG was then lifted out of contact at a rate of 2 mm/min. Under appropriate conditions, the molecules reorient so that the alkyl chains assemble parallel to the HOPG hexagonal lattice (FIG. 1A, top left), optimizing the relatively strong alkyl-Π interactions between molecule and substrate. Monolayers of molecules containing reactive diyne can then be photopolymerized to set the molecular pattern.

Accordingly, after contact with the subphase was broken, HOPG substrates were blown dry with UHP N₂. The HOPG substrates were then placed under a hand-held ultraviolet (UV) lamp (254 nm, 8 W) for 1 hour with about 10 cm between the lamp and the substrate to induce diyne photopolymerization. Conditions used for each amphiphile in this test set are shown in Table 1.

TABLE 1
Test Set 1 - Amphiphile solution volume and target
MMA for L-S transfer of each amphiphile.

| | Volume | Target MMA |
|---|---|---|
| Amphiphile | (μL) | (Å²/chain) |
| TCD-NH₂ | 25 | 54 |
| PCD-NH₂ | 36 | 40 |
| TCD-OH | 20 | 34 |
| TCD-COOH | 35 | 34 |

In initial experiments, sPDAs assembled from TCD-NH₂ (FIG. 6A, structure at top) were utilized. Molecules assembled head-to-head, generating lines of functional head groups about 1 nm in width. Energy-minimized models (FIG. 6A, bottom) illustrate an edge-to-edge lamellar width of 5.3 mm (FIG. 6A, right) for the polymerized system, in good agreement with the 5.6 nm periodicity observed by AFM in FIG. 6B. While sPDAs are typically studied at nanoscopic scales (<1 μm) by AFM, scanning electron microscopy (SEM) enables the characterization of molecular ordering over microscopic scales (3-500 μm), commensurate with the scale of fluorescence microscopy images used for characterization on hydrogel surfaces. In SEM images, stripe phase monolayers locally increase secondary electron generation, creating brighter features in the SEM image that contrast with darker bare HOPG. Molecular orientation in the monolayer is revealed by cracking defects along the lamellar axis (visible in FIGS. 6C and 6D) that accumulate due to conformational changes during striped phase polymerization (compare lamellar width in FIG. 6A).

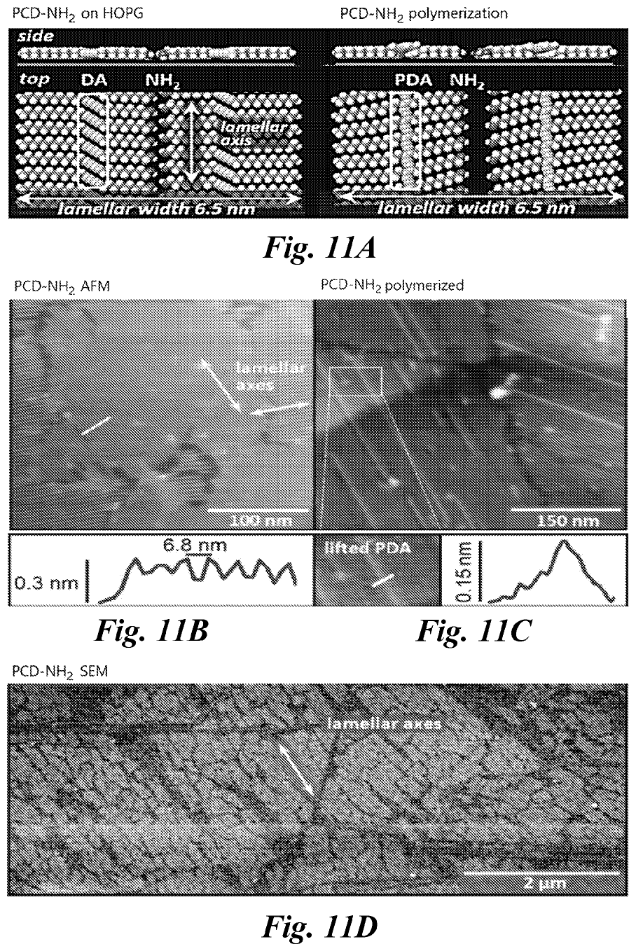

In other tests where the amphiphiles comprised PCD-NH₂, the amphiphiles lying on HOPG ordered into a repeating striped (lamellar) pattern, which generated 1-nm-wide stripes of functional head groups embedded within the layer of exposed alkyl chains, with a periodicity of about 6.5 nm (FIG. 11A). Irradiation with UV light induced topochemical polymerization of the ordered diacetylene (DA) moieties (FIG. 11B). Both striped pattern and polymerization were visible at nanoscopic scales in AFM images (FIGS. 11C and 11D) and at larger scales using scanning electron microscopy (SEM) (FIG. 11E).

Patterns of GlcNAc for controlled multivalent binding were also prepared by generating monolayers of TCD-GlcNAc on HOPG (FIG. 3A) through L-S conversion. Briefly, the molecules were ordered as a standing phase Langmuir film on an aqueous subphase, and a heated HOPG substrate was then lowered horizontally into contact with the Langmuir film causing a subset of the molecules to reorder from the standing phase into a striped lamellar phase. This arrangement formed about 1.7 nm wide stripes of GlcNAc comprising two opposing rows of GlcNAc, with alky chains on both edges, ca. 7 nm in width (FIGS. 2A and 2B). As shown in the atomic force microscopy (AFM) image of FIG. 2B, the measured lamellar periodicity of 6.7 nm is in good agreement with the modeled structure (right portion (TCD-GlcNAc polymerized) of FIG. 2A). The size scale of molecular domains formed in this way can exceed length scales that are readily imaged by AFM; however, previously we have illustrated that SEM can be used to visualize long-range ordering of molecular domains as shown in FIG. 2B.

TCD-GLcNAc monolayers were prepared using the L-S conversion method described herein in Gyorgydeak et al. (2004), supra using a MicroTrough XL Langmuir-Blodgett trough (Kibron Inc., Helsinki, Finland). Films of each amphiphile were assembled at the air-water interface by depositing a solution (0.75 mg/mL) of desired amphiphile in appropriate solvent (Table 1) on a subphase of Milli-Q water (about 18 MO-cm) at 30° C. For 4,6-TCD-GlcNAc, 10 mM NaOH was used as a subphase. The solvent was allowed to evaporate (15 minutes for chloroform and 30 minutes for methanol) before the trough barriers were slowly moved inward (6-6.23 mm/min barrier motion) until the target MMA was achieved.

TABLE 1
Amphiphile solution volume and target MMA for
Langmuir-Schaefer transfer of each amphiphile.

| | concen- | | | |
|---|---|---|---|---|
| | tration | volume | | MMA |
| amphiphile | (μg/mL) | (μL) | solvent | (Å²/molecule) |
| 10,12-TCD-GlcNAc | 1 | 28 | methanol | 43 |
| 4,6-TCD-GlcNAc | 1 | 45 | methanol | 18 |
| 10,12-TCD-GlcA | 1 | 28 | methanol | 35 |
| 10,12-TCD-OH | 0.75 | 28 | chloroform | 45 |
| 10,12-TCD-COOH | 0.75 | 34.5 | chloroform | 35 |

HOPG substrates were freshly cleaved, mounted on a custom-built, thermally controlled dipping apparatus (Hayes et al. (2017), supra) with the HOPG oriented nearly parallel to the air-water interface (cleaved side facing down), then lowered horizontally at a constant rate of 6 mm/min. The temperature of substrates was held at 65° C. After 2 minutes of contact with the interface, the HOPG was gently lifted out of contact with the liquid at the same speed. After contact with the interface was broken, the substrates were blown dry with UHP N₂. The resulting monolayers were then photopolymerized by UV irradiation (254 nm) for 1 hour with about 10 cm between the lamp and the substrate to induce diyne photopolymerization.

TCD-GlcNAc monolayers were prepared under L-S conditions that induced formation of microscopic vacancies. The procedure was significantly similar to those the present inventors demonstrated for other diyne amphiphiles. Davis et al., Hierarchically patterned noncovalent functionalization of 2D materials by controlled Langmuir-Schaefer conversion, Langmuir 34: 1353-1362 (2018). Briefly, to induce formation of circular vacancies in the monolayer, the dipper speed and substrate temperature were decreased to 2 mm/min and 25° C., respectively. These microscopic patterns are useful as internal standards in assays for binding affinity and specificity of WGA to clusters of carbohydrates embedded in striped phase monolayers. The size scale of vacancies formed in this way exceeded length scales that are readily imaged by AFM; however, SEM can be used to visualize long-range ordering of molecular domains as shown in FIG. 2C. Hayes et al. (2017), supra.

### Example 4

### Selectivity of WGA Towards TCD-GlcNAc Monolayers

Previous studies by others have shown that, in solution, WGA binds with GlcNAc specifically in comparison with other carbohydrates. Monsigny et al., Eur J Biochem 104(1): 147-153 (1980). To assess the selectivity of WGA interaction with TCD-GlcNAc monolayers, PAAm was functionalized with amphiphiles having head groups representing the individual functional groups common in glycans: a hydroxyl group (TCD-OH) and a carboxylic acid (TCD-COOH or TCDA). Additionally, PAAm substrates were functionalized with sPDAs generated from a glucuronic acid (Glc) amphiphile (TCD-GlcA), with a carbohydrate head group that was structurally similar to the GlcNAc, but not a target for WGA binding.

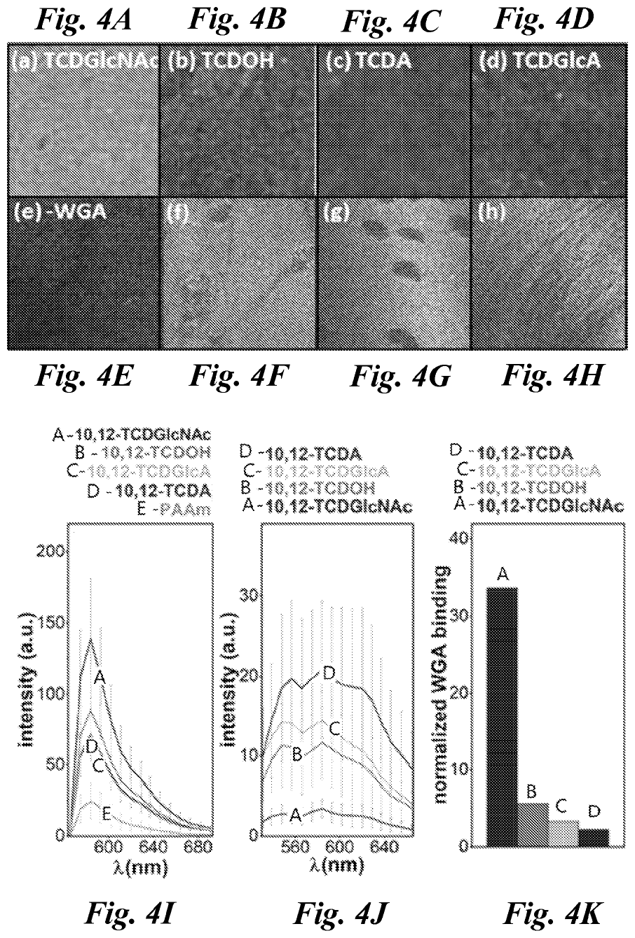

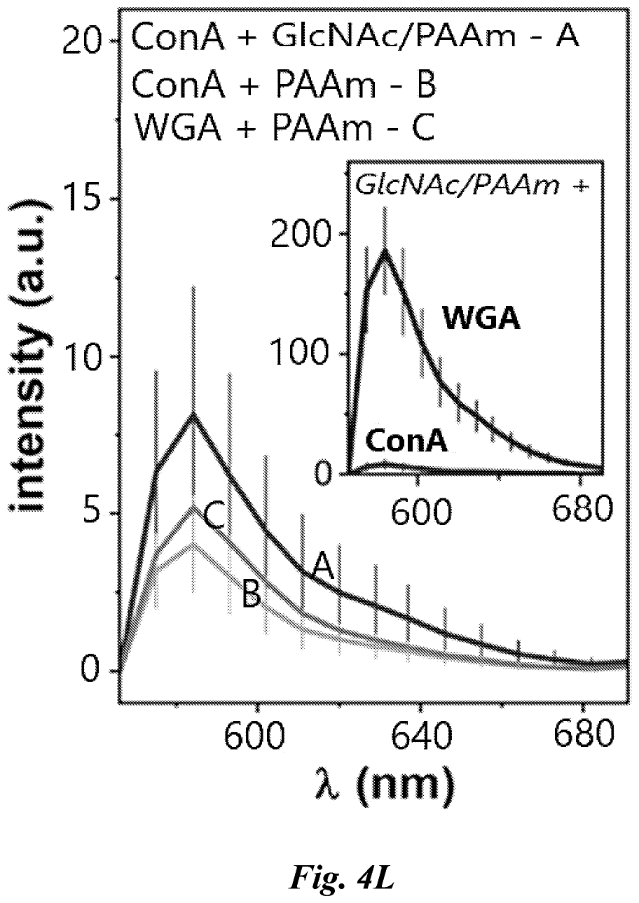

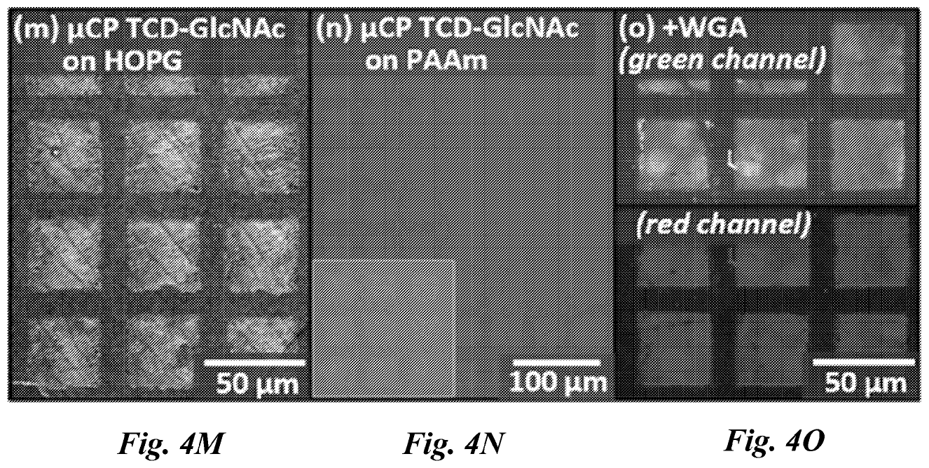

sPDA-functionalized PAAm surfaces were exposed to WGA (5 μg/mL). Greater emission intensity from TCDGlcNAc-functionalized PAAm was observed (FIG. 4A) as compared to other functionalized substrates (FIGS. 4B-4D) exposed to WGA, which supports the preferential binding of WGA to TCDGlcNAc. Interestingly, this increased binding occurred in spite of the relatively low transfer efficiency of TCDGlcNAc in comparison with other surface chemistries.

To control for differences in level of surface functionalization, PDA emission was quantified from monolayers before (FIG. 4J) and after WGA exposure (FIG. 4I; note the larger intensity scale following WGA exposure). It has been shown that differences in sPDA emission intensity relate to the efficiency of sPDA transfer from HOPG to PAAm. Fluorescence emission intensity from TCDGlcNAc monolayers was substantially lower than that for TCDOH, TCDGlcA and TCDA monolayers. Thus, the interfacial concentration of GlcNAc on PAAm was lower in comparison to other functional groups, by up to a factor of 10 in comparison with TCDA, and about five times in comparison with TCDOH and TCDGlcA.

To establish a ratio of WGA binding normalized for surface concentration of the functionalization moiety, maximum emission intensity (I₅₈₄) of WGA-treated monolayers was divided by emission intensity at the PDA emission peak (I₅₈₄). FIG. 4K shows WGA binding normalized for surface coverage using this approach. The normalized WGA binding for TCD-GLcNAc monolayer was 6-(TCD-OH) to 15-(TCDA) times stronger than other functional moieties, while binding to GlcA is similar to that observed for the OH and COOH patterns.

Conversely, the functionalized surfaces did not increase the adsorption of ConA (FIG. 4L); exposure of fluorescence ConA to both TCD-GlcNAc-functionalized (red trace) and unfunctionalized (light red trace) PAAm produces similar fluorescence emissions, also comparable to that observed from WGA exposed to unfunctionalized PAAm (blue trace). Therefore, it appears that the interaction between WGA and the TCD-GlcNAc monolayer is highly selective.

To illustrate the capability to control lectin-carbohydrate interactions across geometrically controlled areas of a soft surface, square patterns of TCD-GlcNAc sPDAs on HOPG were generated by microcontact printing (μCP) (FIG. 4M). After transfer to PAAm, the squares were visible in fluorescence images (FIG. 4N; inset shows enhanced contrast); a larger area of the substrate is shown in this panel to facilitate visualization of the relatively faint pattern. Square patterns became clearly visible in both green and red channels of widefield epifluorescence images after exposure to WGA (FIG. 4O).

### Example 5

### Measurement of Surface Dissociation Constant (K_{D})

In principle, varying the length of the alkyl chain segment joining the carbohydrate to the sPDA backbone represents a means of controlling local concentration of carbohydrates along the stripe. In this context, short alkyl chain segments, which decrease the volume available to each carbohydrate, would be expected to produce higher local concentration (effective molarity) of carbohydrates and potentially decrease K_{D} for a given surface coverage of the same carbohydrate.

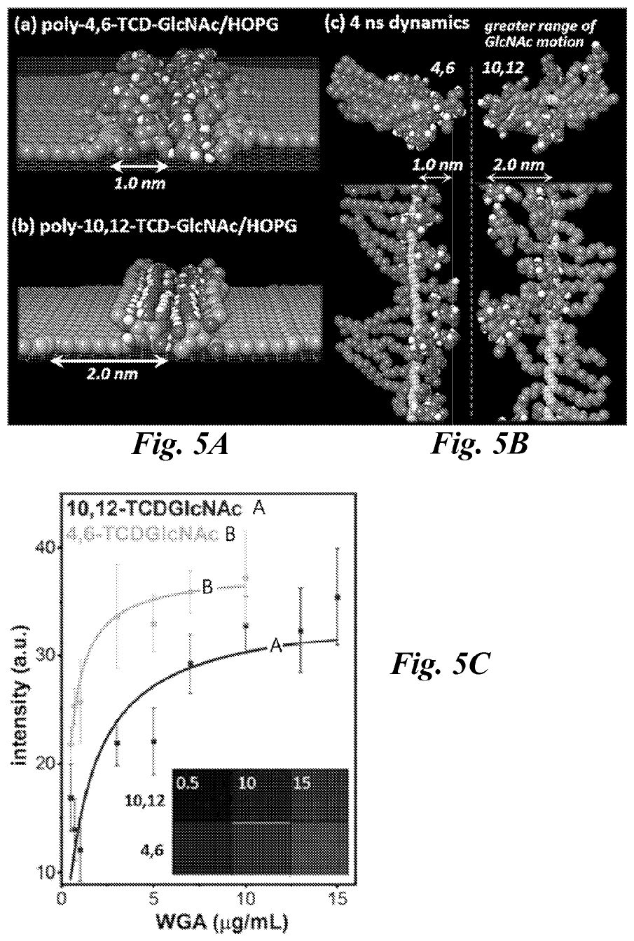

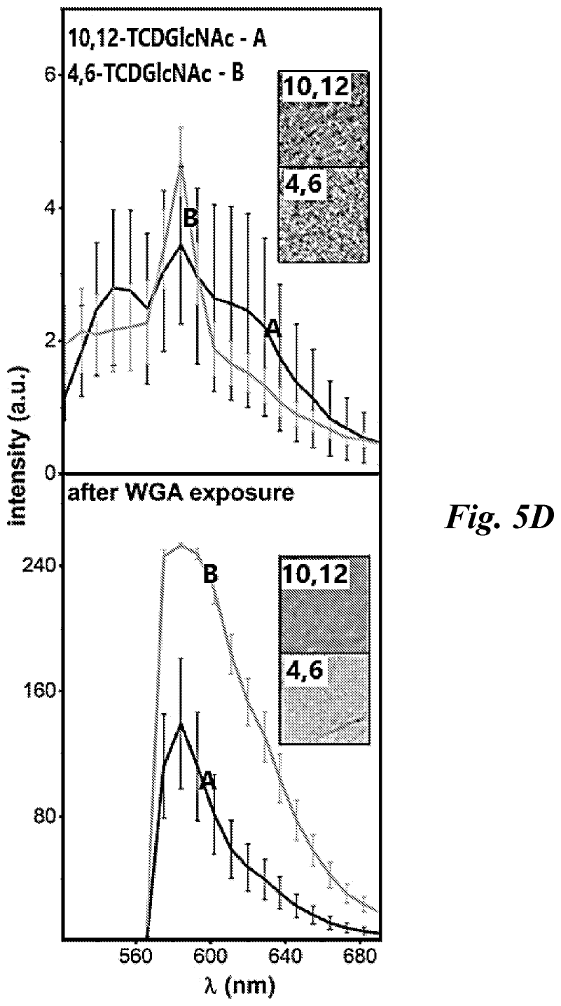

To evaluate the effect of proximal chain length on GlcNAc spatial distribution, models of two isomers of TCD-GlcNAc were created with different proximal chain lengths: 4,6-TCD-GlcNAc (3-carbon proximal chain) and 10,12-TCD-GlcNAc (9-carbon proximal chain). Minimized molecular models of polymerized 4,6-TCD-GlcNAc and 10,12-TCD-GlcNAc monolayers on HOPG (FIG. 5A) illustrate the potential for restrictions on the trajectories of GlcNAc head groups based on the position of the PDA (highlighted in white). The distance from the end of the GlcNAc to the PDA was about 1.0 nm for 4,6-GlcNAc, increasing to about 2.0 nm for the 10,12-TCD-GlcNAc.

To better simulate the possible arrangement of GlcNAc when the PDA is suspended above a porous PAAm substrate, sequential 1 ns molecular dynamics runs were performed in explicit water, with the HOPG substrate removed form the model and the PDA backbone frozen (FIG. 5B). This set of constraints was intended to mimic the periodic covalent linkage of the relatively stiff PDA (persistence length about 16 nm) to the PAAm mesh, while allowing for the motion of pendant alkyl chain segments and head groups. Models after dynamics illustrated that GlcNAc head groups in the 4,6-TCD-GlcNAc layer were more tightly clustered in comparison with those models of 10,12-TCD-GlcNAc. Distances between pyranose rings along each PDA were evaluated, pooling 4 and 8 ns timepoints (FIG. 5E), producing median values of 11.8 and 10.1 Å for C2-OH and C6-CO, respectively, for 4,6-TCD-GlcNAc. Equivalent calculations for 10,12-TCD-GlcNAc produced values of 13.8 and 12.5 Å, respectively. Although differences in median values are modest, the dotted gray tie line illustrates the distance between paired carbohydrate recognition sites on WGA (13 Å). Notably, due to a combination of the lower median values and tighter distribution of values for 4,6-TCD-GlcNAc, a much larger fraction of the distances between adjacent carbohydrates falls within this range (69 and 87%, respectively for C2-OH and C6-CO in the 4,6-TCD-GlcNAc models, in comparison with values of 46 and 54% for the 10,12-TCD-GlcNAc models). The differences in lengths of the alkyl linkers connecting the PDA and carbohydrate head groups for 4,60 and 10,12-TCD-GlcNAc may also contribute to differences in affinity in ways not directly related to local concentration effects, for instance, by influencing the range of possible orientations for the carbohydrate head groups.

To evaluate the effect of proximal chain length on Glc-NAc-WGA binding avidity experimentally, binding to 10,12-TCD-GlcNAc and 4,6-TCD-GlcNAc (see Scheme 5 for structure and synthesis). The transfer efficiency of both isomers to PAAm was similar within error, as measured by PDA fluorescence emission (FIG. 5D, top). Following exposure to WGA (5 μg/mL), a large increase in fluorescence emission was observed for carbohydrate-functionalized surfaces, particularly near 584 nm, the emission maximum for rhodamine label appended to WGA. Spectral distribution also changed to resemble that for the rhodamine label. Emission intensity from 4,6-TCD-GlcNAc+WGA monolayers was substantially greater than that from 10,12-TCD-GlcNAc+WGA monolayers at 5 μg/mL (FIG. 5D, bottom), consistent with the prediction of increased binding to the more localized GlcNAc moieties.

Past studies have reported K_{D} for monovalent interactions between WGA and GlcNAc in the micromolar to millimolar range, and multivalent interactions in the nanomolar range. Wright, Crystallographic elucidation of the saccharide binding mode in wheat germ agglutinin and its biological significance, J Molecular Biology 141: 267-291 (1980); and Kristiansen et al., Quantitative studies of the binding of wheat germ agglutinin (WGA) to chitin-oligosaccharides and partially N-acetylated chitosans suggest inequivalence of binding sites, Carbohydrate Polymers 38: 23-32 (1999). To evaluate whether WGA interaction with TCD-GlcNAc sPDAs were multivalent or monovalent, TCD-GlcNAc/PAAm surfaces were exposed to increasing concentrations of rWGA, maintaining the surface density of GlcNAc in the sPDA constant. The magnitude of emission intensity depends on the active interaction between GlcNAc and rWGA. To calculate K_{D}, the WGA concentration was plotted against the emission intensity and data were fit to a Languir isotherm:

$$F = {F}_{max} [ W G A ] / \{ [ W G A ] + {K}_{D} \} ,$$

where Fₘₐₓ is the maximum fluorescence intensity, a measure of the GlcNAc and WGA interactions, [WGA] is the WGA concentration, and K_{D} is the equilibrium dissociation constant for surface GlcNAc and WGA. Liang et al., Quantitative analysis of carbohydrate-protein interactions using glycan microarrays: determination of surface and solution dissociation constants, J Am Chemical Soc'y 129: 11177-11184 (2007).

The emission intensity increased linearly as [WGA] increased from 0.5 μg/mL (FIG. 5C) indicating an increase in the number of active interactions. As the [WGA] increased further to 15 μg/mL, the emission intensity did not change substantially, denoting the saturation of GlcNAc ligands on the surface. The value of K_{D} obtained by WGA binding curve (36.05±1.30 nM) was consistent with multivalent binding on the surface.

### Example 6

### Transfer of sPDA Monolayers from HOPG to Polymeric Materials

The possibility of transferring sPDAs from HOPG to PAAm polymeric materials using in situ curing of the bulk polymer, analogous to the process demonstrated previously for the transfer to PDMS, was also examined.

Polyacrylamide is generated through a free-radical polymerization of an acrylamide (AAm) monomer, bis(acrylamide) (Bis) crosslinker, ammonium persulfate (APS) radical initiator, and TEMED radical stabilizer. The surface-templated glycopolymers were covalently transferred to PAAm using a covalent transfer strategy in which PAAm is cured in contact with the carbohydrate sPDA on HOPG. This process is effective in transferring sPDAs with simple carboxylic acid or amine head groups to PDMS or to PAAm. Davis et al. (2021), supra. Here, this strategy was leveraged to generate much higher-complexity functional patterns to generate selective multivalent binding on the amorphous hydrogel surface.

Surfaces were grafted with TMS-PMA in accordance with the procedures reported in Yuk et al. (2016), supra. PAAm hydrogels were prepared using the procedures reported in Tse & Engler (2010), supra.

Briefly, AAm and Bis were mixed with a radical initiator and radical stabilizer, poured into rubber molds 2 cm in diameter and 2 mm thick (Ted Pella, Inc.) with an 0.5 cm×0.5 cm square cut to fit the HOPG area. Double sided clear tape (3M Scotch) was used to create a temporary seal between the HOPG and the rubber mold, preventing hydrogel precursor solution from leaking.

To prepare the monomer/crosslinker precursor solution, acrylamide (AAm) (8% w/w) and bis-acrylamide (Bis) (2% w/w) were dissolved together in 5 mL of Milli-Q water, using compositions described herein based on the total concentration of AAm/Bis (w/v) in the gel mixture (% T), and based on the crosslinker-to-monomer ratio (% C):

$$\begin{matrix}\% T = \frac{AAm ( g ) + Bis ( g )}{\text{Total vol} ( mL )} \times 100 \\ \% C = \frac{Bis ( g )}{AAm ( g ) + Bis ( g )} \times 100\end{matrix}$$

The precursor solution was degassed by bubbling with UHP N₂ for five minutes prior to gelation. 10% w/v solution of the APS initiator was prepared in Milli-Q water. To initiate gelation, 10 μL of the APS solution, 1 μL of TEMED, and 989 μL of the AAm/Bis precursor solution were mixed by gentle pipetting, and vortexing thoroughly for 30 seconds. Subsequently 100 μL of the precursor mixture were cast on the prepared HOPG substrates, which were then immediately capped with a 1 cm² vinyl-functionalized glass coverslip, ensuring that the entire HOPG surface was covered by the AAm mixture, and allowed to undergo a free-radical reaction, which set the PAAm and covalently linked it to both vinyl groups on the glass slide and to the PDA backbones in the monolayer. Hydrogels were cured at 60° C. for 15 minutes and exfoliated from the HOPG along with the glass coverslip. Solutions were ultrasonicated for at least 15 minutes (or until all the solid was dissolved) and then sealed with parafilm and stored at room temperature.

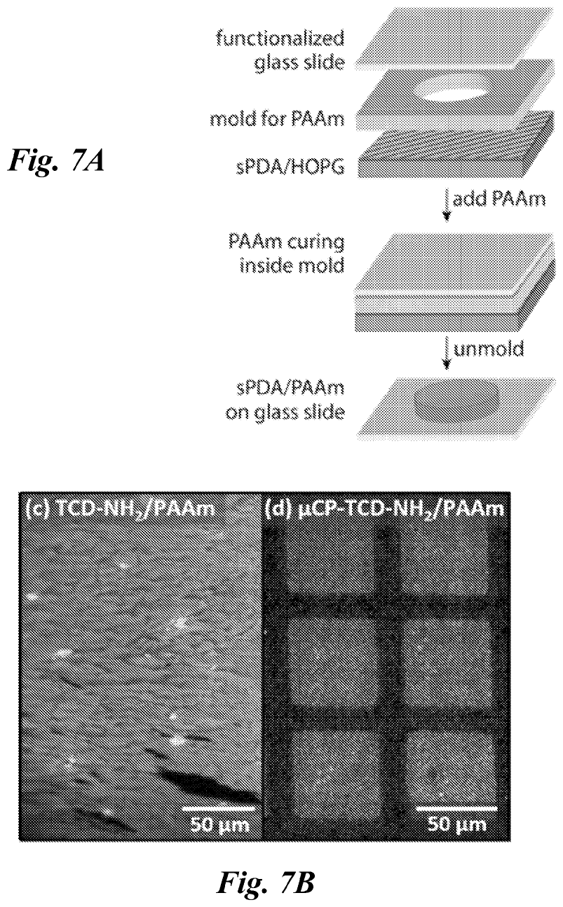

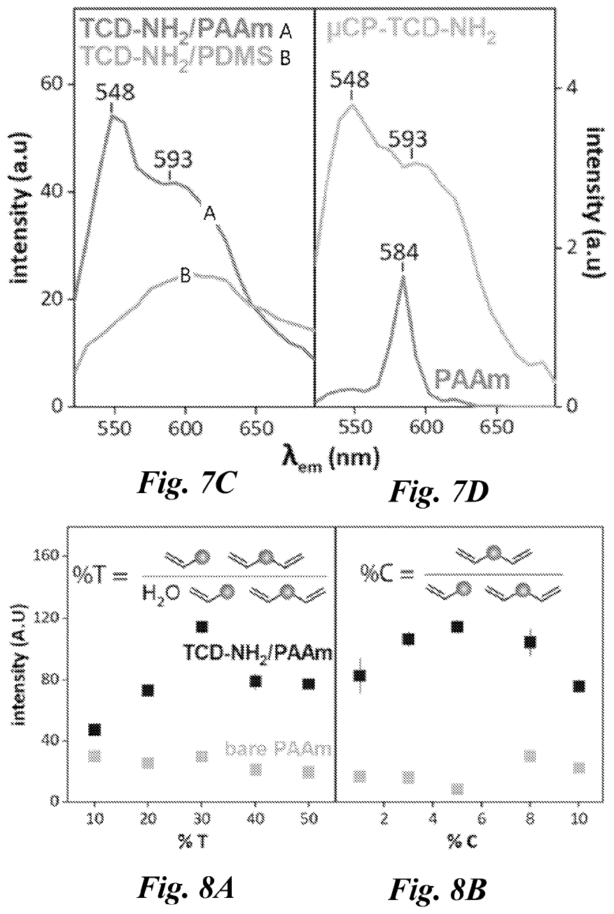

After crosslinking, the PAAm gel was gently exfoliated from the HOPG substrate (FIG. 3A), simultaneously exfoliating any sPDA elements covalently linked to the bulk PAAm network. Because uncured PAAm mixture has low/viscosity relative to PDMS mixtures used previously, transfer was carried out by placing a mold with a circular cutout atop the sPDA/HOPG, adding the PAAm mixture, and placing a functionalized glass slide on top of the stack (FIG. 7A). Maximal transfer was achieved with a total acrylamide concentration of 30-40% and 5% (m/m) fraction of Bis relative to AAm.

Following in situ PAAm curing and exfoliation, confocal spectroscopy (FIG. 3B) and microscopy (FIG. 3C) illustrated surface morphological features and spectral emission features characteristic of sPDA transfer. To provide an internal standard for WGS binding in later experiments, sPDA monolayers were assembled under conditions that resulted in circular vacancies (similar to those visible in the SEM image of FIG. 2C). Following transfer, similar surface features were visible in fluorescence emission.

Previously, the present inventors have shown that sPDA fluorescence emission can be used to quantify the extent of sPDA transfer to PAAm. FIGS. 7B and 7C illustrate this process using both full sPDA monolayers and ssquare patterns of TCD-NH₂ sPDAs assembled on HOPG by microcontact printing (μCP) and then transferred. Fluorescence emission spectra acquired over areas with transferred sPDAs (FIG. 7C) can be fitted to a primary peak near 548 nm, and a set of longer-wavelength peaks, similar to those previously observed for sPDAs transferred to PDMS. Shi et al. (2022), supra. In contrast with PDMS, which exhibits background fluorescence at 514 nm, PAAm exhibits a peak near 584 nm (FIG. 7D, PAAm trace). μCP-TCD-NH₂ fluorescence intensities are lower.

While the spectral characteristics for 10,12-TCD-GlcNAc emission were similar to those observed previously for other sPDAs with simpler head group chemistries, emission intensity was lower than those for full TCD-NH₂ sPDA layers (FIGS. 7C and 7D), indicating the likelihood of lower transfer efficiency and consistent with the smaller average molecular domain sizes achieved by μCP in comparison with L-S transfer, and the previously observation that transfer efficiencies increase with increasing sPDA length. Shi et al. (2022), supra.

To assess whether the functionalized PAAm surfaces yield specificity for carbohydrates known to bind WGA, monolayers of TCD-GlcNAc and TCD-GlcA were assembled and photopolymerized (FIGS. 2D-2E). GlcNAc is known to bind to WGA, while GlcA does not. GlcNAc and GlcA differ at two substituents on the pyranose ring (FIG. 2E, left): at C2, GlcNAc has a bulky acetamido (—NHAc) group, while GlcA has a smaller OH; at C6, GlcNAc has an OH group, and GlcA has a COOH group.

Minimized molecular models of the two sPDAs indicated that C6 is one of the pyranose ring positions that mediates interactions between opposing rows of headgroups along the lamellar median, with GlcA forming strong H-bonded COOH dimers and GlcNAc forming weaker H-bonds between OH groups. Experimentally, it was observed that TCD-GlcNAc forms smaller ordered molecular domains (typical domain sizes 200-300 nm, FIG. 2F, top right) on HOPG in comparison with TCD-GlcA (700-900 nm, FIG. 2F, bottom right) and other amphiphiles with simpler head groups. This would be consistent with the weaker inter-OH H-bonding and possibly also the presence of the bulky acetomido group.

Following in situ PAAm curing and exfoliation, confocal spectroscopy and microscopy (FIGS. 2D and 2E, far right) illustrate surface morphological features and spectral emission features characteristic of sPDA transfer, including a primary emission peak centered at 548 nm and sidebands extending up to about 640 nm. While the spectral characteristics for TCD-GlcNAc emission were similar to those previously observed for other sPDAs with simpler headgroup chemistries, emission intensity is somewhat lower, pointing to the possibility of lower transfer efficiency. TCD-GlcA transferred more efficiently, as evidenced based on greater emission intensity, which is consent with previously work in which the data supported that monolayer chemistries that produce longer sPDAs also resulted in more efficient transfer.

### Example 7

### Impact of Polyacrylamide Gel Structure on sPDA Transfer

A series of experiments were carried out to examine transfer efficiency in relation to PAAm composition. Recently, the relationship between sPDA polymer length and the efficiency of transfer to PDMS was examined, parameterizing the per-unit reaction probability with the PDA, and the number of linkages to the PDA required to exfoliate it with the bulk PDMS. Shi et al. (2022), supra. The previous models were developed by comparing transfer efficiency for molecules with known populations of polymer lengths. Overall, these measurements suggest a 1-2% probability of reaction per PDA unit (P_{rxn,PDMs}), and minimum of 2-3 covalent linkages typically required for exfoliation of each sPDA.

PAAm polymerization is different than PDMS crosslinking, in that each AAm or Bis monomer can react with the PDA. PAAm gel structure is typically controlled by varying the percentage of reactive acrylate (% T) in the reaction mixture, and the percentage of the acrylate that is crosslinker (% C) pursuant to the formula described in Example 3 above. Here, acrylate percentages (% T) from 10-50% and crosslinker percentages (% C) from 1-10%, spanning the range of compositions common for DNA and protein electrophoresis, were used. Polyacrylamide Gel Electrophoresis, In Cold Spring Harbor Protocols, Cold spring Harbor Laboratory Press: 2020.

Overall, it was observed that as T % increases from 10% to 30%, fluorescence emission at λ_{max,PDA} increased by a factor of three, then decreased as % T increased further (40 and 50%). Similarly, emission at λ_{max,PDA} reached a maximum at % C=5%, decreasing by a factor of approximately two at % C=1% and 10%. The observed maximum at 5% crosslinker is in line with previous electron microscopy observations of pore structure, which have indicated a minimum in pore size in approximately that range, for acrylate percentages near those used here. Ruchel et al., Transmission-electron microscopic observations of freeze-etched polyacrylamide gels, J Chromatography 166: 563-575 (1978).

Samples were prepared for cryogenic electron microscopy (cryoEM) by being placed in 10% v/v ethanol/Milli-Q water for 10 minutes; ethanol was used as a cryoprotectant to minimize crystalline ice formation during cryo-fixation. Aston et al., Evaluation of the impact of freezing preparation techniques on the characterization of alginate hydrogels by cryo-SEM, Eur. Polym J 82: 1-15 (2016). Samples were then prepared by cutting irregular edges off, producing flat surfaces with final dimensions of ca. 0.5 cm×0.5 cm. Each sample was then mounted on a flat specimen holder with optimal cutting temperature (OCT) cryo embedding media. Mounted samples in the specimen holder were clamped to an ALT118 brass Gatan plunger (Gatan, Oxford, UK) and immersed in a liquid nitrogen slush under ambient pressure until equilibrated. Samples were then cryo-transferred into the Gatan Alto 2500 preparation chamber, preset to −180° C. Cross-sectional samples were prepared using a vise clamp holder and a surgical scalpel to fracture the sample at the base. Subsequently, the sample was transferred onto the Nova SEM cryo stage, which was pre-cooled to −85° C. for sublimation of ice. Sublimation was monitored until the sample surface was frost-free. When sublimation was complete, samples were returned to the cryo preparation chamber for sputter-coating with a Pt target for 45-60 seconds; a 45-second sputtering time was used, unless otherwise specified.

Following cryo-fixation and sputtering, samples were imaged using a FEI Nova Nano SEM (FEI, Thermo Fisher Scientific, Hillsboro, OR). The SEM chamber temperature was maintained at −140° C., and imaging was performed using a 5 kV beam with a spot size of 5 mm. Electrons were collected using an Everhart Thornley Detector for low magnification images, and an immersion-mode through-the-lens detector for high magnification images.

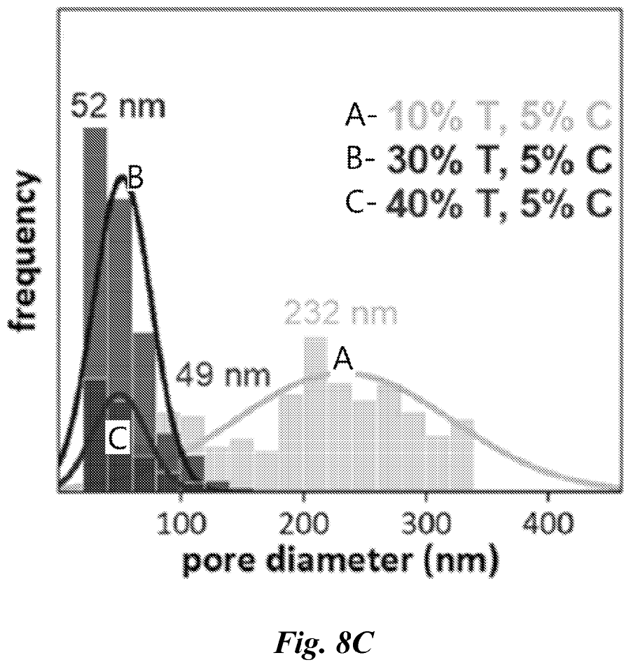

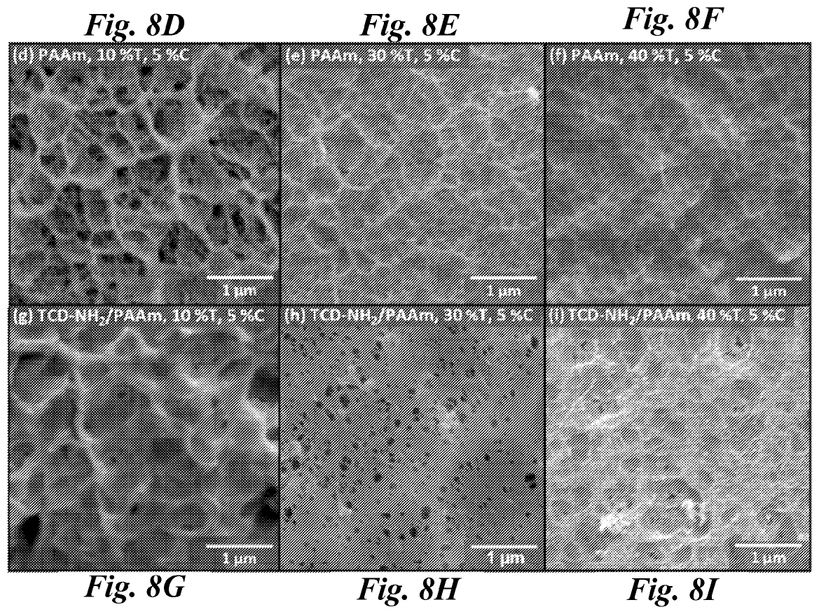

Analysis of cryoEM images estimates surface pore diameters at about 50 nm for 30 and 40% T gels, which produce higher degrees of transfer (FIG. 8C). CryoEM images of unfunctionalized PAAm (FIGS. 8D-8F) and TCD-NH2/PAAm (FIGS. 8G-8I) illustrate smoothing of the gel surface associated with the transfer of the TCD-NH₂ sPDA monolayer, particularly for PAAm compositions that result in high degrees of sPDA transfer based on fluorescence emission. These differences likely reflect changes in the polymerized network at the surface that include not only the sPDA, but PAAm oligomers that react with the sPDA, changing the near-surface pore structure. At larger scales, the relationship between the surface layer and the underlying gel structure is in some cases evident based on tearing near the edges.

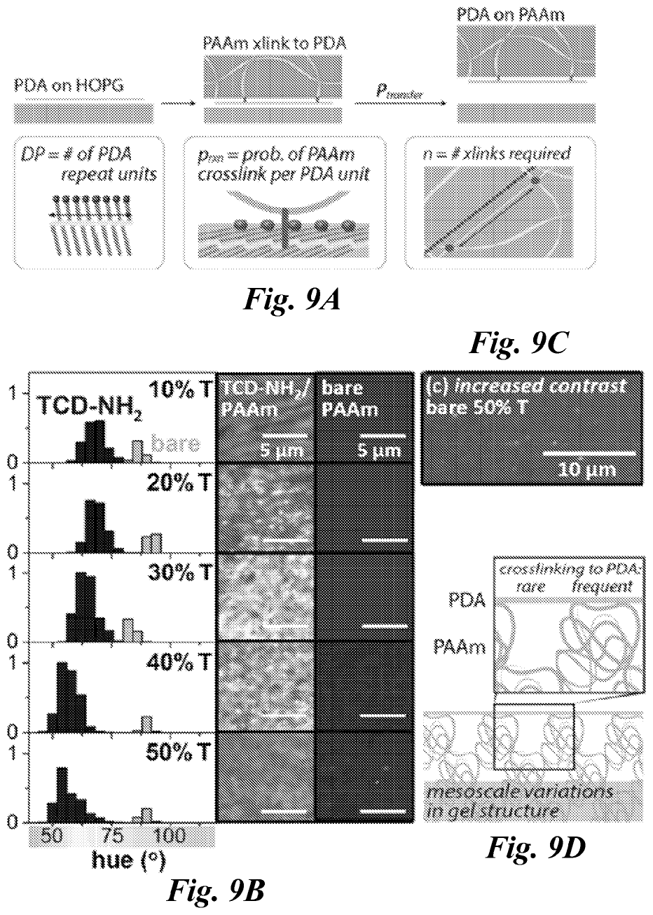

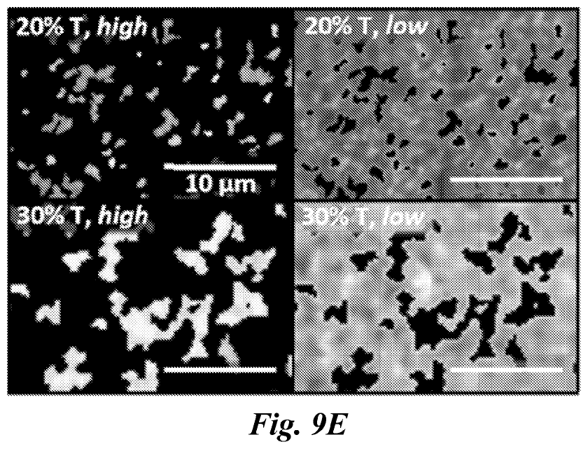

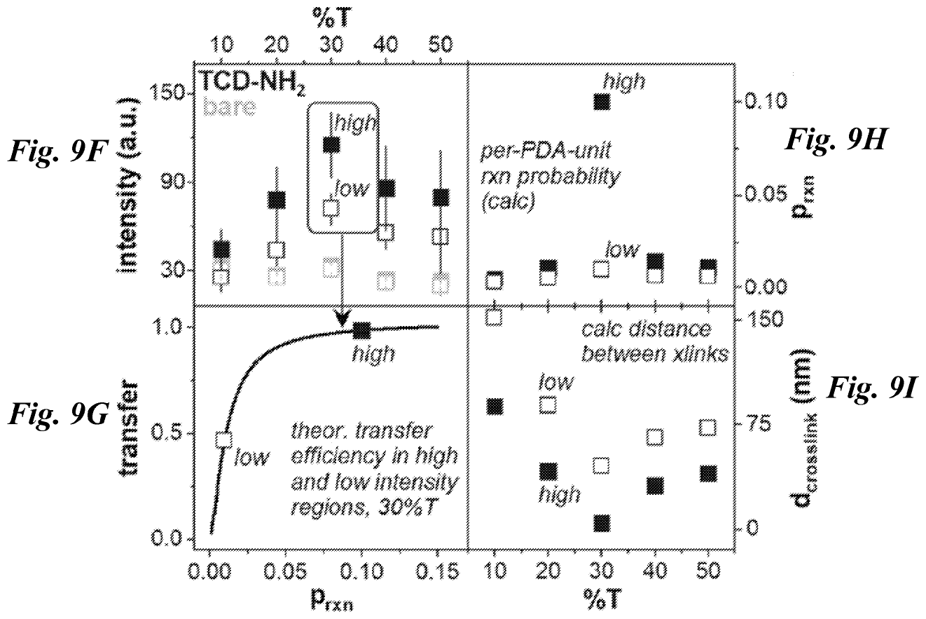

Heterogeneities in the porous architecture (FIGS. 8A-8I) could have the potential to impact sPDA transfer by varying local reactant concentrations, and thus p_{rxn,AAm} (FIG. 9A). Previous literature has demonstrated that in these polymerization reactions, early stages of the reaction produce higher-density particles rich in Bis crosslinker, with diameters on the order of hundreds of nm. Righetti & Caglio, On the kinetics of monomer incorporation into polyacrylamide gels, as investigated by capillary zone electrophoresis, Electrophoresis 14: 573-582 (1993); and Stellwagen, Electrophoresis of DNA in agarose gels, polyacrylamide gels and in free solution, Electrophoresis, 30: S199-S195 (2009). In the final stages of the reaction, particles then become loosely crosslinked to other particles through fibers rich in AAm. Weiss et al., Influence of polymerization initiation rate on permeability of aqueous polyacrylamide gels, J Polymer Science, Polymer Phys. Ed 19: 1505-1512 (1981); and Weiss & Silberberg, Inhomogeneity of polyacrylamide gel structure from permeability and viscoelasticity, Br. Polym J 9: 144-150 (1977). Increases in crosslinker or acrylate group concentration can produce more tightly crosslinked networks in the initial stage of polymerization, producing larger voids. Weiss et al. (1981), supra; Stellwagen (2009), supra; Stellwagen, Apparent pore size of polyacrylamide gels: comparison of gels cast and run in tris-acetate-EDTA and tris-borate-EDTA buffers, Electrophoresis 19: 1542-1547 (1998); Margolis & Wrigley, Improvement of pore gradient electrophoresis by increasing the degree of crosslinking at high acrylamide concentrations, J Chromatography 106: 204-209 (1975); Campbell et al., Electrophoresis of small proteins in highly concentrated and crosslinked polyacrylamide gradient gels, Analytical Biochemistry 129: 31-36 (1983).

In the context of PDA transfer, PAAm network heterogeneities could result in local strong crosslinking to the PDA monolayer in some regions, and more limited crosslinking in others. Visual inspection of fluorescence images of TCD-NH₂/PAAm illustrate speckling patterns that vary with % T (FIG. 9B). Unfunctionalized PAAm produced much weaker fluorescence emission overall; however, speckling was visible for 40 and 50% T gels (FIG. 9C), at scales similar to those observed for functionalized surfaces. These observations may be consistent with the interpretation that one source of heterogeneities in sPDA transfer is heterogeneities in local PAAm gel structure (FIG. 9D).

Relative intensities of longer- and shorter-wavelength PDA emission peaks also shift with increasing % T (FIG. 9B, histograms), producing visible differences in color, plotted here using a cylindrical coordinate system that converts red, blue, and green components into linear coordinates from 0 to 360. Reasons for these shifts are not currently well-understood, but may relate to differences in torsion on the PDA backbone based on crosslinking density.

To quantify local differences in sPDA fluorescence intensity, images were segmented into high- and low-intensity pixel sets (segmentation examples for 20 and 40% T in FIG. 9E), and fluorescence intensities were separately extracted for high- and low-intensity pixels (FIG. 9F). Models of transfer developed previously for PDMS were used (FIGS. 9A and 9G) to relate these differences in PDA emission to differences in transfer and to estimate differences in sPDA crosslinking to the PAAm network (FIGS. 9H and 9I).

The models of the present disclosure for sPDA crosslinking and exfoliation onto a bulk polymer do not require knowledge of the chemical nature of the bulk polymer. The model relates the per-PDA-unit probability of reaction with the bulk polymer (pᵣₓₙ) and number of crosslinks required for transfer (n) to the percentage of the sPDA monolayer projected to transfer (P_{transfer}, FIG. 9A), given the population of observed sPDA polymer lengths (DP) on HOPG. Here, TCD-NH₂ sPDAs, which are structurally equivalent to those used in the previous study (same DP), were used and the same number of crosslinks were expected to be required to exfoliate the sPDAs (same n). Thus, an observed similar extent of transfer (calculated based on PDA fluorescence emission) would indicate that the model would still predict the per-PDA reaction probability (pᵣₓₙ).

If it is hypothesized that the brightest regions observed in TCD-NH₂/PAAm transfer experiments (FIG. 9D, filled blue markers) correspond to complete monolayer transfer, high-intensity regions in 30% T and 40% T transfers represent nearly complete transfer. Low-intensity regions (empty blue markers) in the same samples have emission intensities of about 35% those of the high-intensity regions, after correcting for background emission from bare PAAm (yellow markers), and would represent, on average, about 35% transfer. Low and high-intensity pixels were mapped onto the transfer model developed previously (FIG. 9E), which related transfer efficiency with pᵣₓₙ. FIG. 9F shows calculated pᵣₓₙ values for high and low-intensity regions across the tested range of % T; FIG. 9G shows the theoretical transfer efficiency for 40% T/5% C formulation based on values in FIG. 9F. This approach suggests that in regions with the highest transfer efficiency, pᵣₓₙ may be as high as 0.1, corresponding to an average distance between crosslinks of 10 monomers (5 nm), in highly crosslinked areas. However, in the low-intensity areas of similar samples, this method predicts about 50 nm between crosslinks, similar to average PAAm pore diameters calculated from cryoEM images in FIG. 8C (FIGS. 9F and 9I).

### Example 8

### Using Amine Striped Phases to Control DATA Adsorption

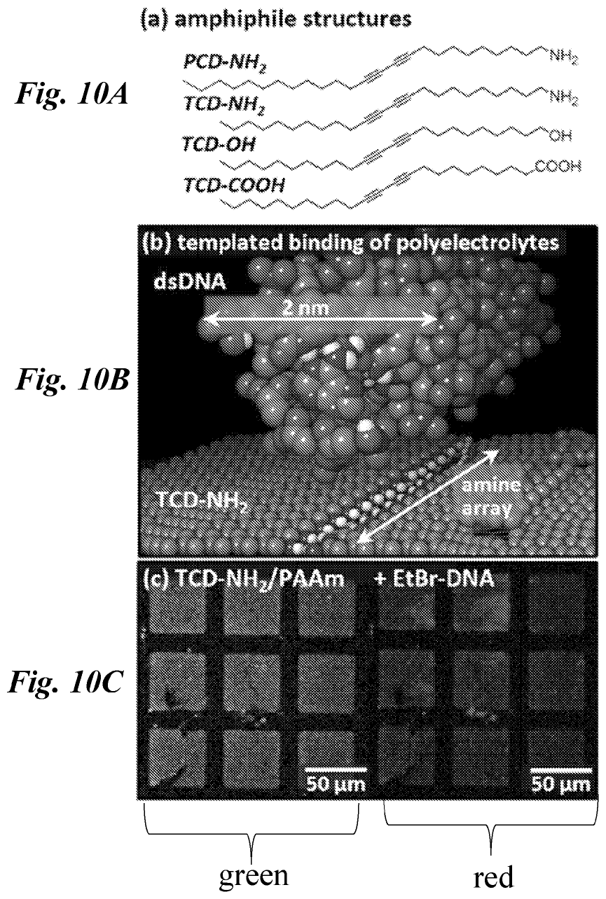

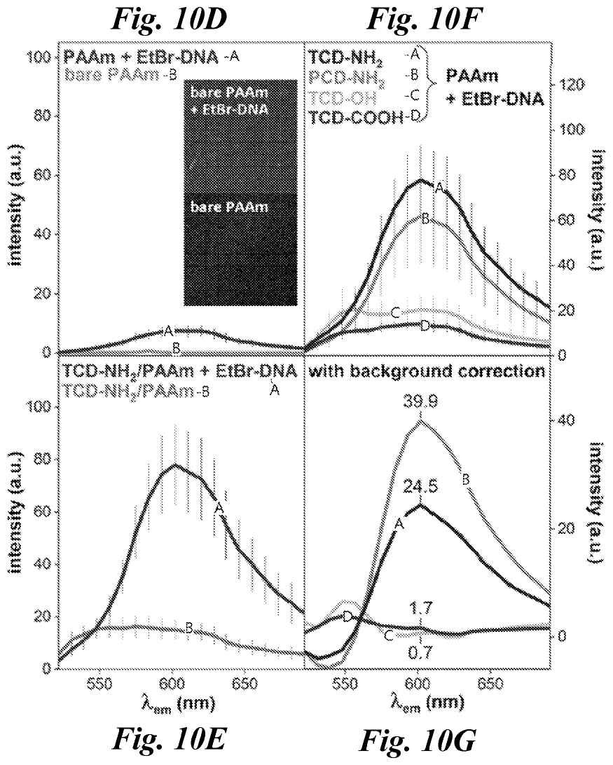

The linear patterns of amines on the hydrogel surface offer a means of controlling adsorption, and perhaps ultimately orientation, of complex biomolecules including DNA (FIGS. 10A-10G). Two amine sPDAs, as well as amphiphiles with carboxylic acid and hydroxyl head groups, were tested (structures shown in FIG. 10A). The model in FIG. 10B illustrates the relative scale of a double-stranded DNA (dsDNA) helix and the sPDA surface pattern. The capacity of the surfaces to control DNA adsorption was examined using long dsDNA isolated from salmon sperm, with helix lengths ranging up to 2 million base pairs. DNA was first incubated with the intercalating fluorophore ethidium bromide (EtBr), to produce fluorescent labeling along the contour length of each helix.

Initially, a 1 mg/mL stock solution (2500 μM) of EtBr in Milli-Q water was prepared and diluted to 100 μM with Milli-Q water. The ratio of dsDNA sodium salt from salmon testes (average 2000 base pairs, equivalent to 680 nm double strand helix contour length) to EtBr was adjusted to produce a molar ratio of one dsDNA strand per 80 EtBr molecules (DNA-EtBr). For example, to prepare a 1 μM DNA and 80 M EtBr solution, 1.36 mg of dsDNA were weighed into a vial along with 800 μL of a 100 μM EtBr solution and 200 μL of Milli-Q water to make a final volume of 1 mL. The solution was mixed thoroughly by vortex for at least 10 minutes and allowed to equilibrate overnight. DNA solutions were stored at 4° C. when not in use.

To quantify DNA-EtBr adsorption on sPDA/PAAm substrates, 30 μL of DNA-EtBr solution were cast on functionalized or unfunctionalized PAAm substrates and incubated for 10 min in a petri dish protected from light and dust. Following incubation, the EtBr-DNA solution was carefully wicked off the PAAm surface using a Kimwipe. Samples were rinsed using the procedure set forth in Das et al., Comparison of the nonspecific binding of DNA-conjugated gold nanoparticles between polymeric and monomeric self-assembled monolayers, Langmuir 25: 235-241 (2009) and comprised of: two 20-second cycles of vortexing with 0.1% w/v SDS in Milli-Q water, followed by two 20-second cycles of vortexing with pure Milli-Q water. Samples were subsequently stored in Milli-Q water and analyzed within one hour after exposure. FIG. 10C shows μCP-TCD-NH₂/PAAm before (left) and after (right) exposure to EtBr-DNA, illustrating the appearance of red fluorescence in the amine-patterned areas.

To quantify binding of DNA to the surface-patterned PAAm, fluorescence emission spectra were acquired from bare and functionalized PAAm with and without exposure to EtBr-DNA. FIG. 10D illustrates that exposure of bare PAAm to EtBr-DNA, followed by a standard washing protocol produces modest fluorescence emission near 600 nm, in the spectral region associated with EtBr emission. FIG. 10D (inset) illustrates swatches of bare PAAm without (top) and with (bottom) exposure to EtBr. FIG. 10E shows emission from TCD-NH₂/PAAm without (green) and with (red) exposure to EtBr-DNA, with the same vertical scale used for the bare PAAm graph in FIG. 10D.

EtBr-DNA binding to a set of sPDA-functionalized PAAm surfaces (monomer structures shown in FIG. 10A) was tested to assess the role of functional group type and density on the surface. Following exposure to EtBr-DNA at the same concentration (FIG. 10F), amine-functionalized surfaces (TCD-NH₂ and PCD-NH₂) exhibited much greater DNA adsorption than hydroxyl (TCD-OH) or carboxylic acid (TCD-COOH) functionalized surfaces. FIG. 10G shows emission spectra with the monolayer and PAAm background subtracted, illustrating the up to 40-fold increase in emission intensity at the EtBr maximum associated with amine-functionalized surfaces in contrast with OH or COOH-functionalized surfaces.

### Example 9

### Secondary Functionalization of sPDAs with Azides

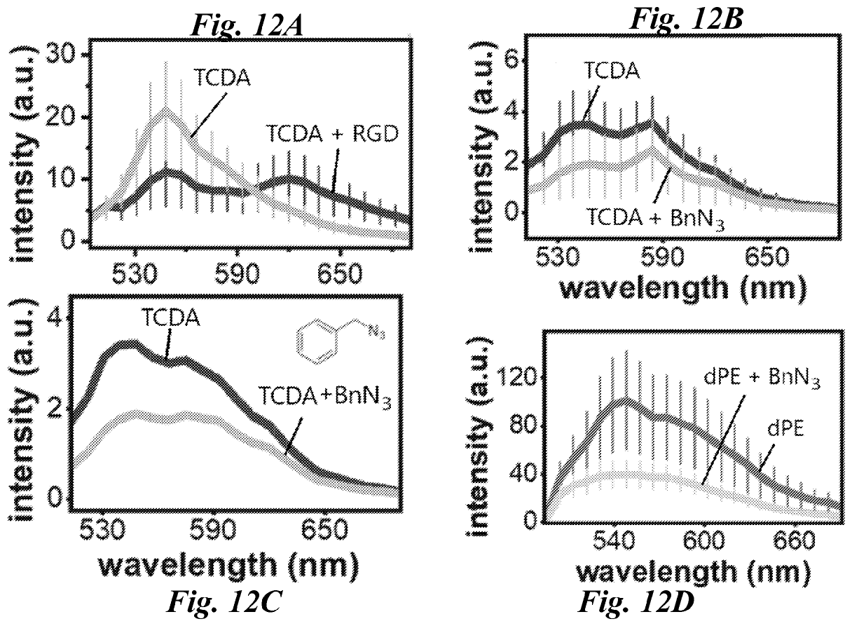

Surface functionalized with sPDAs were then exposed to azides (which can undergo a click reaction with an alkyne) to produce an sPDA/PAAm surface with secondary functionalities installed along the sPDA backbone as shown in FIG. 1C. TCDA/PAAm was first reacted with cyclic arginine-glycine-aspartic acid (RGD; a cell adhesion peptide) azide (cRGD-N₃) and with Bn-N₃, a structurally simpler azide which limits other surface interactions. Following exposure to each azide for 4 hours, there was an about 50% decrease in the PDA fluorescence emission near λmax (FIGS. 12A-12C), consistent with extensive reaction with the azide.

To examine whether this change could be due to an unexpected interaction with the COOH head groups in the TCDA sPDA, Bn-N3 was also exposed to diyne phosphoethanolamine (dPE) sPDA/PAAm surfaces, in which the monomer has a zwitterionic head group comprised of a phosphate/ammonium pair. Here again, there was a notable decrease in sPDA fluorescence emission (about 60% after two hours exposure) (FIG. 12D).

### Example 10

### Cell Culture on Functionalized and Unfunctionalized PAAm Surfaces

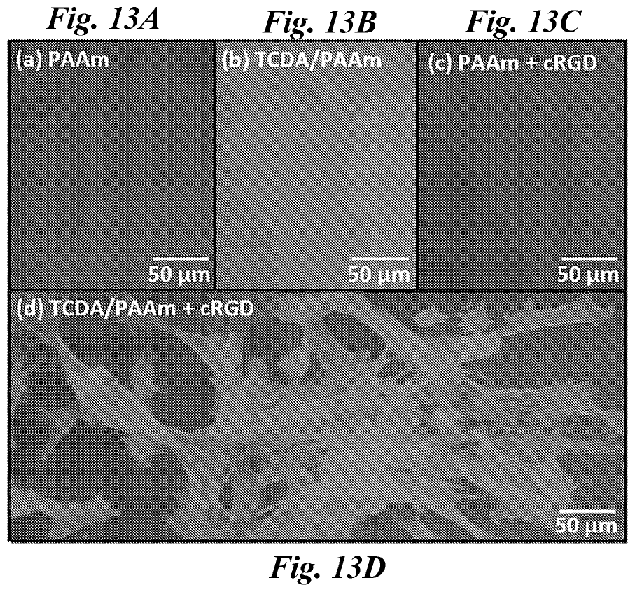

To examine the impacts of cRGD functionalization on cell adhesion, C2C12 murine myoblasts were cultured on a set of unfunctionalized and functionalized PAAm surfaces. Previously, the present inventors observed that both amine and phosphoethanolamine (PE) sPDAs increase myoblast adhesion, while carboxylic acid sPDAs do not. Here, cells were cultured with bare PAAm (FIG. 13A), TCDA/PAAm (FIG. 13B), bare PAAm exposed to cRGD under the click conditions described above (FIG. 13C), and TCDA/PAAm+cRGD (FIG. 13D). Out of these four conditions, only TCDA/PAAm+cRGD resulted in significant C2C12 myoblast adhesion.

1. A scaffold comprising a molecular layer positioned on a surface of a support material, the molecular layer comprising polymerized amphiphiles organized in a striped pattern to form an exposed polymer backbone, each of the polymerized amphiphiles comprising one or more exposed alkyl chains and one or more functional head groups covalently bonded with an exposed alkyl chain;
wherein the one or more functional head groups are in an orientation capable of binding a target and the alkyl chains and functional head groups are organized in a repeating striped pattern comprising a width at a periodicity.

2. (canceled)

3. The scaffold of claim 1, wherein the polymerized amphiphiles of the molecular layer are organized in a lying-down orientation substantially parallel to the surface of the support material, whereas the one or more exposed alkyl chains and the one or more functional head groups are not so restricted.

4. The scaffold of claim 1, wherein the support material comprises a hydrogel.

5-7. (canceled)

8. The scaffold of claim 1, wherein the molecular layer is covalently bonded to the surface of the support material and/or comprises a monolayer.

9. (canceled)

10. The scaffold of claim 1, wherein the periodicity of the pattern is tunable by adjusting a length of the one or more alkyl chains.

11. The scaffold of claim 1, wherein the one or more functional head groups are polar head groups and/or each of the one or more exposed alkyl chains is a nonpolar alkyl chain.

12. (canceled)

13. The scaffold of claim 1, wherein the amphiphiles are polymerized through polymerizable functional groups within the one or more alkyl chains and the polymerizable functional groups comprise polymerizable diacetylene (DA) groups.

14-15. (canceled)

16. The scaffold of claim 1, wherein the one or more functional head groups comprise carbohydrate (CHO) head groups, a carboxylate, an amine, an alcohol, a peptide, a substrate of an enzyme, or any combination of two or more of the foregoing.

17. The scaffold of claim 16, wherein the CHO type are selected, and the pattern is designed, such that the CHOs act together as a component of an extracellular matrix; and/or
the one or more functional head groups comprise a monosaccharide, a glycan, a N-acetyl-D-glucosamine (GlcNAc), a glucuronic acid (GlcA), a N-acetyl-D-neuraminic acid, or any combination of two or more of the foregoing.

18. (canceled)

19. The scaffold of claim 1, wherein the polymerized amphiphiles comprise C₅-C₃₀ functionalized amphiphiles with an internal diyne.

20. The scaffold of claim 19, wherein the polymerized amphiphiles comprise 10,12-tricosadiynamine (10,12-TCD-NH₂), 4,6-tricosadiynamine (4,6-TCD-NH₂), 10,12-pentacosadiynamine (10,12-PCD-NH₂), 4,6-pentacosadiynamine (4,6-PCD-NH₂), or any combination of the foregoing.

21. (canceled)

22. The scaffold of claim 1, wherein repeating elements of the striped pattern are linear and substantially parallel to each other.

23. The scaffold of claim 1, wherein the molecular layer comprises a multi-valent binding surface.

24-27. (canceled)

28. The scaffold of claim 1, wherein the target is a cell, a biomolecule, or a bioactive molecule.

29-30. (canceled)

31. The scaffold of claim 1, wherein individual ordered regions of the polymerized amphiphiles have linear dimensions of greater than about 100 nm in length while maintaining nanoscale periodicity of the striped pattern of functional head groups.

32-34. (canceled)

35. The scaffold of claim 1, wherein the periodicity is about 5 nm to about 10 nm (e.g., 5-10 nm).

36. (canceled)

37. The scaffold of claim 1, wherein the support material has a pore diameter from about 1 nm to about 200 nm.

38. The scaffold of claim 1, wherein the exposed polymer backbone is covalently bonded to an azide-functionalized molecule or a thiol-functionalized molecule in the absence of copper.

39-42. (canceled)

43. A cell or tissue culture system comprising the scaffold of claim 1.

44. A multivalent receptor assay comprising the scaffold of claim 1.

45. The multivalent receptor assay of claim 44 comprising a lectin assay.

46-99. (canceled)
